SEQUENCE LISTING (53 sequences)

SEQ ID NO: 1 - protein, 20 aa, Artificial sequence
  Synthetic
METDTLLLWVLLLWVPGSTG

SEQ ID NO: 2 - protein, 20 aa, Artificial sequence
  Synthetic
MSRSVALAVLALLSLSGLEA

SEQ ID NO: 3 - protein, 19 aa, Artificial sequence
  Synthetic
AVGQDTQEVIVVPHSLPFK

SEQ ID NO: 4 - protein, 49 aa, Artificial sequence
  Synthetic
ASAKPTTTPAPRPPTPAPTIASQPLSLRPEAARPAAGGAVHTRGLDFAK

SEQ ID NO: 5 - protein, 6 aa, Artificial sequence
  Synthetic
GAPGAS

SEQ ID NO: 6 - protein, 17 aa, Artificial sequence
  Synthetic
GAPGSGGGGSGGGGSAS

SEQ ID NO: 7 - protein, 5 aa, Artificial sequence
  Synthetic
GGGGS

SEQ ID NO: 8 - protein, 33 aa, Artificial sequence
  Synthetic
ASESKYGPPCPPCPAVGQDTQEVIVVPHSLPFK

SEQ ID NO: 9 - protein, 39 aa, Artificial sequence
  Synthetic
ASGGGGSGELAAIKQELAAIKKELAAIKWELAAIKQGAG

SEQ ID NO: 10 - protein, 14 aa, Artificial sequence
  Synthetic
ASESKYGPPCPPCP

SEQ ID NO: 11 - DNA, 1536 bp, Artificial Sequence
  Synthetic
atgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccatacaagtcaaaatgcccaagagtcacaaggctattcaagcagacggttggatgtgtcatgcttccaaatgggtcactacttgtgatttccgctggtatggaccgaagtatataacacagtccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaa

SEQ ID NO: 12 - protein, 511 aa, Artificial Sequence
  Synthetic
MKCLLYLAFLFIGVNCKFTIVFPHNQKGNWKNVPSNYHYCPSSSDLNWHNDLIGTAIQVKMPKSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 13 - protein, 495 aa, Unknown
  WT VSV-G envelop protein
KFTIVFPHNQKGNWKNVPSNYHYCPSSSDLNWHNDLIGTAIQVKMPKSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 14 - DNA, 1536 bp, Artificial sequence
  Synthetic
atgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgccccagagtcacaaggctattcaagcagacggttggatgtgtcatgcttccaaatgggtcactacttgtgatttccgctggtatggaccgaagtatataacacagtccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaagccgaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaa

SEQ ID NO: 15 - protein, 511 aa, Artificial sequence
  Synthetic
MKCLLYLAFLFIGVNCKFTIVFPHNQKGNWKNVPSNYHYCPSSSDLNWHNDLIGTALQVKMPQSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 16 - protein, 495 aa, Artificial sequence
  Synthetic
KFTIVFPHNQKGNWKNVPSNYHYCPSSSDLNWHNDLIGTALQVKMPQSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 17 - protein, 495 aa, Artificial sequence
  Synthetic
KFTIVFPHNQKGNWKNVPSNYHYCPSSSDLNWHNDLIGTAIQVKMPQSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 18 - DNA, 1818 bp, Artificial Sequence
  Synthetic
atgggcagccggatcgtgatcaaccgggagcacctgatgatcgaccggccctacgtgctgctggccgtgctgttcgtgatgttcctgagcctgatcggcttgctagccattgctggaatccggctgcacagagccgccatctacaccgccgagatccacaagagcctgagcaccaacctggacgtgaccaacagcatcgagcatcaggtcaaggacgtgctgacccccctgtttaagatcatcggcgacgaagtgggcctgcggaccccccagagattcaccgacctggtcaagttcatcagcgacaagatcaagttcctgaaccccgaccgggagtacgacttccgggacctgacctggtgcatcaacccccccgagcggatcaagctggactacgaccagtactgcgccgatgtggccgccgaggaactgatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaaccaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctatctcttcacagtcccaattaaggaagcaggcggagactgccatgccccaacatacctacctgcggaggtggatggtgatgtcaaactcagttccaatctggtgattctacctggtcaagatctccaatatgttttggcaacctacgatacttcccgggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatgactacaaagacgatgacgacaagtga

SEQ ID NO: 19 - protein, 605 aa, Unknown
  Exemplary wild-type measles envelope protein
MGSRIVINREHLMIDRPYVLLAVLFVMFLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLETRTTNQFLAVSKGNCSGPTTIRGQFSNMSLSLLDLYLGRGYNVSSIVTMTSQGMYGGTYLVEKPNLSSKRSELSQLSMYRVFEVGVIRNPGLGAPVFHMTNYLEQPVSNDLSNCMVALGELKLAALCHGEDSITIPYQGSGKGVSFQLVKLGVWKSPTDMQSWVPLSTDDPVIDRLYLSSHRGVIADNQAKWAVPTTRTDDKLRMETCFQQACKGKIQALCENPEWAPLKDNRIPSYGVLSVDLSLTVELKIKIASGFGPLITHGSGMDLYKSNHNNVYWLTIPPMKNLALGVINTLEWIPRFKVSPYLFTVPIKEAGGDCHAPTYLPAEVDGDVKLSSNLVILPGQDLQYVLATYDTSRVEHAVVYYVYSPSRSFSYFYPFRLPIKGVPIELQVECFTWDQKLWCRHFCVLADSESGGHITHSGMVGMGVSCTVTREDGTNDYKDDDDK

SEQ ID NO: 20 - DNA, 1818 bp, Artificial Sequence
  Synthetic
atgggcagccggatcgtgatcaaccgggagcacctgatgatcgaccggccctacgtgctgctggccgtgctgttcgtgatgttcctgagcctgatcggcttgctagccattgctggaatccggctgcacagagccgccatctacaccgccgagatccacaagagcctgagcaccaacctggacgtgaccaacagcatcgagcatcaggtcaaggacgtgctgacccccctgtttaagatcatcggcgacgaagtgggcctgcggaccccccagagattcaccgacctggtcaagttcatcagcgacaagatcaagttcctgaaccccgaccgggagtacgacttccgggacctgacctggtgcatcaacccccccgagcggatcaagctggactacgaccagtactgcgccgatgtggccgccgaggaactgatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaaccaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtcccgcgctcttcaatgtcccaattaaggaagcaggcggagactgccatgccccaacatacctacctgcggaggtggatggtgatgtcaaactcagttccaatctggtgattctacctggtcaagatctccaatatgttttggcaacctacgatacttccgcggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatgactacaaagacgatgacgacaagtga

SEQ ID NO: 21 - protein, 605 aa, Artificial sequence
  Synthetic
MGSRIVINREHLMIDRPYVLLAVLFVMFLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLETRTTNQFLAVSKGNCSGPTTIRGQFSNMSLSLLDLYLGRGYNVSSIVTMTSQGMYGGTYLVEKPNLSSKRSELSQLSMYRVFEVGVIRNPGLGAPVFHMTNYLEQPVSNDLSNCMVALGELKLAALCHGEDSITIPYQGSGKGVSFQLVKLGVWKSPTDMQSWVPLSTDDPVIDRLYLSSHRGVIADNQAKWAVPTTRTDDKLRMETCFQQACKGKIQALCENPEWAPLKDNRIPSYGVLSVDLSLTVELKIKIASGFGPLITHGSGMDLYKSNHNNVYWLTIPPMKNLALGVINTLEWIPRFKVSPALFNVPIKEAGGDCHAPTYLPAEVDGDVKLSSNLVILPGQDLQYVLATYDTSAVEHAVVYYVYSPSRSFSYFYPFRLPIKGVPIELQVECFTWDQKLWCRHFCVLADSESGGHITHSGMVGMGVSCTVTREDGTNDYKDDDDK

SEQ ID NO: 22 - DNA, 1785 bp, Artificial sequence
  Synthetic
atgaagaagatcaacgagggcctgctggacagcaagatcctgagcgccttcaacaccgtgattgccctgctgggctctatcgtgatcatcgtgatgaacatcatgatcatccagaactacacccggtccaccgacaaccaggccgtgattaaggatgctctgcagggaatccagcagcagatcaaaggcctggccgacaagatcggcacagagatcggccctaaggtgtccctgatcgacaccagcagcaccatcacaatccccgccaatatcggactgctgggaagcaagatcagccagagcaccgccagcatcaacgagaacgtgaacgagaagtgcaagttcaccctgcctccactgaagatccacgagtgcaacatcagctgccccaatcctctgccattcagagagtacagaccccagacagagggcgtgtccaatctcgtgggcctgcctaacaacatctgcctgcagaaaaccagcaaccagatcctgaagcctaagctgatctcctacacactgcccgtcgtgggccagagcggcacctgtattacagatcctctgctggccatggacgagggctactttgcctacagccacctggaaagaatcggcagctgtagccggggagtgtccaagcagagaatcatcggcgtgggcgaagtgctggatagaggcgacgaagtgcccagcctgttcatgaccaatgtgtggacccctcctaatcctaacaccgtgtaccactgcagcgccgtgtacaacaacgagttctactacgtgctgtgcgccgtgtccacagtgggcgaccctatcctgaacagcacctattggagcggcagcctgatgatgaccagactggccgtgaagcccaagagcaatggcggcggatacaaccagcatcagctggccctgcggtccatcgagaagggcagatacgacaaagtgatgccttacggccccagcggcatcaagcaaggcgataccctgtactttcccgccgtgggatttctcgtgcggaccgagttcaagtacaacgacagcaactgccccatcaccaagtgccagtacagcaagcccgagaactgcagactgagcatgggcatcagacccaacagccactacatcctgagaagcggcctgctgaagtacaacctgagcgacggcgagaaccccaaggtggtgttcatcgagatcagcgaccagcggctgtctatcggcagcccctccaagatctacgactctctgggccagccagtgttctaccaggccagctttagctgggacaccatgatcaagttcggcgacgtgctgaccgtgaatcccctggtggtcaactggcggaacaataccgtgatcagccggcctggccagtctcagtgccccagattcaatacctgtcctgccatttgcgccgaaggcgtgtacaatgacgccttcctgatcgatcggatcaactggatctctgccggcgtgttcctggactctaatgccacagccgccaatcctgtgttcaccgtgttcaaggacaatgagatcctgtatcgggcccagctggcctccgaggacacaaatgcccagaaaacaatcaccaactgctttctgctcaagaacaagatctggtgcatcagcctggtggaaatctacgacaccggcgacaacgtgatcaggcccaagctgttcgccgtgaagatccctgagcagtgtacaggcggcggaggatctggcggaggtggaagcggaggcggtggatctgctagcgattacaaggatgacgacgataagtga

SEQ ID NO: 23 - protein, 594 aa, Artificial sequence
  Synthetic
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCTGGGGSGGGGSGGGGSASDYKDDDDK

SEQ ID NO: 24 - protein, 512 aa, Unknown
  Cocal Virus Glycoprotein
MNFLLLTFIVLPLCSHAKFSIVFPQSQKGNWKNVPSSYHYCPSSSDQNWHNDLLGITMKVKMPKTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTERELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 25 - DNA, 1539 bp, Unknown
  Cocal Virus Glycoprotein
atgaactttctgctgctcacgtttatcgtactcccgttgtgctctcatgcgaaattttcaatagtctttcctcagtcccagaaagggaattggaaaaatgttccctccagttaccactattgtccctcctcctctgaccaaaactggcacaatgacttgctcgggattacaatgaaagtaaagatgccgaaaacccataaagccatacaggcggatgggtggatgtgtcacgctgcgaagtggatcactacatgcgatttccggtggtatggccctaagtacattacacactctatccatagcatacagccgacatcagagcaatgcaaagagagtattaaacagaccaaacaagggacatggatgagccctggctttccacctcagaattgtgggtacgcgaccgtcacggatagtgtcgctgttgtggtgcaggccacgccacatcacgtactcgtagatgaatatactggtgaatggatcgactcccaattcccgaatgggaaatgtgagacggaagagtgcgaaacagtgcataactcaaccgtttggtattccgattacaaggttactggtctttgcgacgccaccctcgtggataccgagatcacgttttttagtgaggatggcaagaaagagtcaataggcaaacctaatactggctaccggagtaactatttcgcttacgagaagggtgacaaggtatgtaaaatgaactattgcaagcatgcgggagtgcgactccccagtggggtatggttcgaatttgttgaccaagacgtatacgccgctgcgaagttgccagaatgccccgtaggcgcgaccatttcagcacctacccaaacgtccgttgacgtctccttgatactggatgtagagcgaatcctggactacagtctctgccaggaaacgtggtcaaaaataagaagtaagcagccagtttcacccgtggatctgtcttatctggcgccaaaaaacccgggcacgggccctgcttttaccataattaacggaacgcttaaatacttcgaaacccgctacattagaatcgatatagacaatcctattatcagcaagatggtagggaagatatctgggtctcaaacggagcgagaattgtggacggagtggttcccttatgagggagtggaaattgggcccaacgggatcctcaagaccccaacgggttacaagttccctctgtttatgatcggccatggcatgttggacagtgacttgcacaaaacatctcaggcagaggttttcgaacatccacatttggcggaggcgcccaagcaacttccagaagaagaaactctcttctttggagatacaggcatttcaaaaaatcctgtagaactgatagaagggtggttctcttcctggaaatcaacggttgtcacgtttttctttgcaataggcgtatttatactcctgtacgtcgtagcccgcattgtgatcgcagtacgatacagataccagggcagtaacaataaacgcatatataatgacatcgaaatgtcaaggttccgaaagtga

SEQ ID NO: 26 - protein, 512 aa, Artificial sequence
  Synthetic
MNFLLLTFIVLPLCSHAKFSIVFPQSQKGNWKNVPSSYHYCPSSSDQNWHNDLLGITMKVKMPQTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEAELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 27 - DNA, 1539 bp, Artificial sequence
  Synthetic
atgaactttctgctgctcacgtttatcgtactcccgttgtgctctcatgcgaaattttcaatagtctttcctcagtcccagaaagggaattggaaaaatgttccctccagttaccactattgtccctcctcctctgaccaaaactggcacaatgacttgctcgggattacaatgaaagtaaagatgccgcagacccataaagccatacaggcggatgggtggatgtgtcacgctgcgaagtggatcactacatgcgatttccggtggtatggccctaagtacattacacactctatccatagcatacagccgacatcagagcaatgcaaagagagtattaaacagaccaaacaagggacatggatgagccctggctttccacctcagaattgtgggtacgcgaccgtcacggatagtgtcgctgttgtggtgcaggccacgccacatcacgtactcgtagatgaatatactggtgaatggatcgactcccaattcccgaatgggaaatgtgagacggaagagtgcgaaacagtgcataactcaaccgtttggtattccgattacaaggttactggtctttgcgacgccaccctcgtggataccgagatcacgttttttagtgaggatggcaagaaagagtcaataggcaaacctaatactggctaccggagtaactatttcgcttacgagaagggtgacaaggtatgtaaaatgaactattgcaagcatgcgggagtgcgactccccagtggggtatggttcgaatttgttgaccaagacgtatacgccgctgcgaagttgccagaatgccccgtaggcgcgaccatttcagcacctacccaaacgtccgttgacgtctccttgatactggatgtagagcgaatcctggactacagtctctgccaggaaacgtggtcaaaaataagaagtaagcagccagtttcacccgtggatctgtcttatctggcgccaaaaaacccgggcacgggccctgcttttaccataattaacggaacgcttaaatacttcgaaacccgctacattagaatcgatatagacaatcctattatcagcaagatggtagggaagatatctgggtctcaaacggaggccgaattgtggacggagtggttcccttatgagggagtggaaattgggcccaacgggatcctcaagaccccaacgggttacaagttccctctgtttatgatcggccatggcatgttggacagtgacttgcacaaaacatctcaggcagaggttttcgaacatccacatttggcggaggcgcccaagcaacttccagaagaagaaactctcttctttggagatacaggcatttcaaaaaatcctgtagaactgatagaagggtggttctcttcctggaaatcaacggttgtcacgtttttctttgcaataggcgtatttatactcctgtacgtcgtagcccgcattgtgatcgcagtacgatacagataccagggcagtaacaataaacgcatatataatgacatcgaaatgtcaaggttccgaaagtga

SEQ ID NO: 28 - protein, 226 aa, Artificial sequence
  Synthetic
MKKTQTWIITCIYLQLLLFNPLVKTKEICGDPVTDNVKDITKLVANLPNDYMITLNYVAGMDVLPSHCWLRDMVIQLSLSLTTLLDKFSNISEGLSNYSIIHKLGIIVDDLFFCMEENAPKNIKEFPKRPETRSFTPEEFFSIFNRSIDAFKDFMVASDTSDCVLSYPYDVPDYAASAVGQDTQEVIVVPHSLPFKVVVISAILALVVLTIISLIILIMLWQKKPR

SEQ ID NO: 29 - DNA, 734 bp, Artificial sequence
  Synthetic
tgtgtgctggcccatcactttggcaaagcacgtgagatctgaattctgacactatgaaaaaaacacaaacttggatcattacttgcatatacctgcaacttctccttttcaacccactcgtcaagaccaaagaaatatgcggcgaccccgtcactgataacgtgaaggatatcaccaaactcgttgctaaccttccaaatgactacatgattacattgaactatgtagcaggaatggacgttcttccatcacattgctggctccgggacatggtaatccagcttagcctcagccttactaccttgctggacaagtttagcaacatttccgaagggttgagtaactatagtattattcacaagctcggtatcatagttgacgacttgttcttctgtatggaagagaatgcacccaaaaatatcaaagaattccccaaaaggcccgaaaccaggtcatttaccccagaagaatttttcagtatttttaatcgctcaatagacgcattcaaggatttcatggttgcttctgacacatctgactgcgtattgtcctatccttacgatgtcccggactatgctgctagcgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttga

SEQ ID NO: 30 - protein, 249 aa, Artificial sequence
  Synthetic
MTVLAPAWSPNSSLLLLLLLLSPCLRGTPDCYFSHSPISSNFKVKFRELTDHLLKDYPVTVAVNLQDEKHCKALWSLFLAQRWIEQLKTVAGSKMQTLLEDVNTEIHFVTSCTFQPLPECLRFVQTNISHLLKDTCTQLLALKPCIGKACQNFSRCLEVQCQPDSSTLLPPRSPIALEATELPEPRPRQYPYDVPDYAASAVGQDTQEVIVVPHSLPFKVVVISAILALVVLTIISLIILIMLWQKKPR

SEQ ID NO: 31 - DNA, 750 bp, Artificial sequence
  Synthetic
atgaccgtacttgctccagcttggagccctaactcctctctccttctgctgttgctgcttctgtccccatgtctgcggggtacccccgactgttatttttctcatagcccaatatctagcaatttcaaagttaagtttcgggagcttaccgatcatttgcttaaggattatccagtaacagtagcagttaatctccaagacgagaaacactgtaaggccttgtggtccctctttcttgcccaacgctggattgagcagcttaagaccgtagctggctcaaaaatgcaaactctcctggaggatgtcaacacagagattcattttgtcacctcctgcacctttcaacctctccctgagtgccttagattcgttcagactaacatttctcacctcctgaaggacacctgcacccagctgcttgctctgaaaccttgcatcggcaaggcatgtcaaaatttctcacggtgtctcgaagtccagtgccagcctgatagttccacattgctccccccaaggtcacccatagcactggaagccactgaacttcccgaaccacgccctcggcagtatccttacgatgtcccggactatgctgctagcgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttga

SEQ ID NO: 32 - protein, 226 aa, Artificial sequence
  Synthetic
MKKTQTWIITCIYLQLLLFNPLVKTKEICGNPVTDNVKDITKLVANLPNDYMITLNYVAGMDVLPSHCWLRDMVIQLSLSLTTLLDKFSNISEGLSNYSIIDKLGKIVDDLVLCMEENAPKNIKESPKRPETRSFTPEEFFSIFNRSIDAFKDFMVASDTSDCVLSYPYDVPDYAASAVGQDTQEVIVVPHSLPFKVVVISAILALVVLTIISLIILIMLWQKKPR

SEQ ID NO: 33 - DNA, 678 bp, Artificial sequence
  Synthetic
atgaaaaaaacccagacctggattattacctgcatttatctgcagctgctgctgtttaacccgctggtgaaaaccaaagaaatttgcggcaacccggtgaccgataacgtgaaagatattaccaaactggtggcgaacctgccgaacgattatatgattaccctgaactatgtggcgggcatggatgtgctgccgagccattgctggctgcgcgatatggtgattcagctgagcctgagcctgaccaccctgctggataaatttagcaacattagcgaaggcctgagcaactatagcattattgataaactgggcaaaattgtggatgatctggtgctgtgcatggaagaaaacgcgccgaaaaacattaaagaaagcccgaaacgcccggaaacccgcagctttaccccggaagaattttttagcatttttaaccgcagcattgatgcgtttaaagattttatggtggcgagcgataccagcgattgcgtgctgagctatccgtatgatgtgccggattatgcggcgagcgcggtgggccaggatacccaggaagtgattgtggtgccgcatagcctgccgtttaaagtggtggtgattagcgcgattctggcgctggtggtgctgaccattattagcctgattattctgattatgctgtggcagaaaaaaccgcgc

SEQ ID NO: 34 - protein, 234 aa, Artificial sequence
  Synthetic
MELTDLLLAAMLLAVARLTLSSPVAPACDPRLLNKLLRDSHLLHSRLSQCPDVDPLSIPVLLPAVDFSLGEWKTQTEQSKAQDILGAVSLLLEGVMAARGQLEPSCLSSLLGQLSGQVRLLLGALQGLLGTQLPLQGRTTAHKDPNALFLSLQQLLRGKVRFLLLVEGPTLCVRYPYDVPDYAASAVGQDTQEVIVVPHSLPFKVVVISAILALVVLTIISLIILIMLWQKKPR

SEQ ID NO: 35 - DNA, 705 bp, Artificial sequence
  Synthetic
atggaattgactgacctgctgttggctgccatgcttcttgccgtcgcccgcttgacactcagctctccagttgctcccgcctgcgatcccaggttgcttaacaaactgcttcgagactctcatctgcttcacagcaggttgtctcaatgtccagacgtggatccactttctattcctgtcctgctgcccgcagttgacttctcattgggagagtggaaaactcagaccgaacaatctaaggcacaagacatattgggcgctgtgtctctgttgctcgaaggcgtcatggctgcccgggggcagcttgaaccctcatgtctctcctccttgctgggtcagctttctggacaagttagattgctgctgggagctttgcaagggttgttgggtacacaactcccacttcagggtcgcactaccgctcacaaagatccaaatgccctttttcttagtcttcaacaattgctgcggggaaaagtgagatttttgttgctggttgaaggaccaacattgtgcgttcgatatccttacgatgtcccggactatgctgctagcgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttga

SEQ ID NO: 36 - protein, 238 aa, Artificial sequence
  Synthetic
MKKTQTWIITCIYLQLLLFNPLVKTKEICGDPVTDNVKDITKLVANLPNDYMITLNYVAGMDVLPSHCWLRDMVIQLSLSLTTLLDKFSNISEGLSNYSIIHKLGIIVDDLFFCMEENAPKNIKEFPKRPETRSFTPEEFFSIFNRSIDAFKDFMVASDTSDCVLSYPYDVPDYAASESKYGPPCPPCPAVGQDTQEVIVVPHSLPFKVVVISAILALVVLTIISLIILIMLWQKKPR

SEQ ID NO: 37 - DNA, 717 bp, Artificial sequence
  Synthetic
atgaaaaaaacacaaacttggatcattacttgcatatacctgcaacttctccttttcaacccactcgtcaagaccaaagaaatatgcggcgaccccgtcactgataacgtgaaggatatcaccaaactcgttgctaaccttccaaatgactacatgattacattgaactatgtagcaggaatggacgttcttccatcacattgctggctccgggacatggtaatccagcttagcctcagccttactaccttgctggacaagtttagcaacatttccgaagggttgagtaactatagtattattcacaagctcggtatcatagttgacgacttgttcttctgtatggaagagaatgcacccaaaaatatcaaagaattccccaaaaggcccgaaaccaggtcatttaccccagaagaatttttcagtatttttaatcgctcaatagacgcattcaaggatttcatggttgcttctgacacatctgactgcgtattgtcctatccttacgatgtcccggactatgctgctagcgaaagcaagtatggtcctccctgccccccgtgcccagctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttga

SEQ ID NO: 38 - protein, 261 aa, Artificial sequence
  Synthetic
MTVLAPAWSPNSSLLLLLLLLSPCLRGTPDCYFSHSPISSNFKVKFRELTDHLLKDYPVTVAVNLQDEKHCKALWSLFLAQRWIEQLKTVAGSKMQTLLEDVNTEIHFVTSCTFQPLPECLRFVQTNISHLLKDTCTQLLALKPCIGKACQNFSRCLEVQCQPDSSTLLPPRSPIALEATELPEPRPRQYPYDVPDYAASESKYGPPCPPCPAVGQDTQEVIVVPHSLPFKVVVISAILALVVLTIISLIILIMLWQKKPR

SEQ ID NO: 39 - DNA, 786 bp, Artificial sequence
  Synthetic
atgaccgtacttgctccagcttggagccctaactcctctctccttctgctgttgctgcttctgtccccatgtctgcggggtacccccgactgttatttttctcatagcccaatatctagcaatttcaaagttaagtttcgggagcttaccgatcatttgcttaaggattatccagtaacagtagcagttaatctccaagacgagaaacactgtaaggccttgtggtccctctttcttgcccaacgctggattgagcagcttaagaccgtagctggctcaaaaatgcaaactctcctggaggatgtcaacacagagattcattttgtcacctcctgcacctttcaacctctccctgagtgccttagattcgttcagactaacatttctcacctcctgaaggacacctgcacccagctgcttgctctgaaaccttgcatcggcaaggcatgtcaaaatttctcacggtgtctcgaagtccagtgccagcctgatagttccacattgctccccccaaggtcacccatagcactggaagccactgaacttcccgaaccacgccctcggcagtatccttacgatgtcccggactatgctgctagcgaaagcaagtatggtcctccctgccccccgtgcccagctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttga

SEQ ID NO: 40 - protein, 246 aa, Artificial sequence
  Synthetic
MELTDLLLAAMLLAVARLTLSSPVAPACDPRLLNKLLRDSHLLHSRLSQCPDVDPLSIPVLLPAVDFSLGEWKTQTEQSKAQDILGAVSLLLEGVMAARGQLEPSCLSSLLGQLSGQVRLLLGALQGLLGTQLPLQGRTTAHKDPNALFLSLQQLLRGKVRFLLLVEGPTLCVRYPYDVPDYAASESKYGPPCPPCPAVGQDTQEVIVVPHSLPFKVVVISAILALVVLTIISLIILIMLWQKKPR

SEQ ID NO: 41 - DNA, 741 bp, Artificial sequence
  Synthetic
atggaattgactgacctgctgttggctgccatgcttcttgccgtcgcccgcttgacactcagctctccagttgctcccgcctgcgatcccaggttgcttaacaaactgcttcgagactctcatctgcttcacagcaggttgtctcaatgtccagacgtggatccactttctattcctgtcctgctgcccgcagttgacttctcattgggagagtggaaaactcagaccgaacaatctaaggcacaagacatattgggcgctgtgtctctgttgctcgaaggcgtcatggctgcccgggggcagcttgaaccctcatgtctctcctccttgctgggtcagctttctggacaagttagattgctgctgggagctttgcaagggttgttgggtacacaactcccacttcagggtcgcactaccgctcacaaagatccaaatgccctttttcttagtcttcaacaattgctgcggggaaaagtgagatttttgttgctggttgaaggaccaacattgtgcgttcgatatccttacgatgtcccggactatgctgctagcgaaagcaagtatggtcctccctgccccccgtgcccagctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttga

SEQ ID NO: 42 - protein, 238 aa, Artificial sequence
  Synthetic
MKKTQTWIITCIYLQLLLFNPLVKTKEICGNPVTDNVKDITKLVANLPNDYMITLNYVAGMDVLPSHCWLRDMVIQLSLSLTTLLDKFSNISEGLSNYSIIDKLGKIVDDLVLCMEENAPKNIKESPKRPETRSFTPEEFFSIFNRSIDAFKDFMVASDTSDCVLSYPYDVPDYAASESKYGPPCPPCPAVGQDTQEVIVVPHSLPFKVVVISAILALVVLTIISLIILIMLWQKKPR

SEQ ID NO: 43 - DNA, 717 bp, Artificial sequence
  Synthetic
atgaaaaaaacacaaacttggatcattacttgcatatacctgcaacttctccttttcaacccactcgtcaagaccaaagaaatatgcggcaaccccgtcactgataacgtgaaggatatcaccaaactcgttgctaaccttccaaatgactacatgattacattgaactatgtagcaggaatggacgttcttccatcacattgctggctccgggacatggtaatccagcttagcctcagccttactaccttgctggacaagtttagcaacatttccgaagggttgagtaactatagtattattgataagctcggtaagatagttgacgacttggttctctgtatggaagagaatgcacccaaaaatatcaaagaatcccccaaaaggcccgaaaccaggtcatttaccccagaagaatttttcagtatttttaatcgctcaatagacgcattcaaggatttcatggttgcttctgacacatctgactgcgtattgtcctatccttacgatgtcccggactatgctgctagcgaaagcaagtatggtcctccctgccccccgtgcccagctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttga

SEQ ID NO: 44 - protein, 226 aa, Artificial sequence
  Synthetic
MKKTQTWILTCIYLQLLLFNPLVKTEGICRNRVTNNVKDVTKLVANLPKDYMITLKYVPGMDVLPSHCWISEMVVQLSDSLTDLLDKFSNISEGLSNYSIIDKLVNIVDDLVECVKENSSKDLKKSFKSPEPRLFTPEEFFRIFNRSIDAFKDFVVASETSDCVVSYPYDVPDYAASAVGQDTQEVIVVPHSLPFKVVVISAILALVVLTIISLIILIMLWQKKPR

SEQ ID NO: 45 - DNA, 680 bp, Artificial sequence
  Synthetic
tgaagaagactcagacctggattctgacgtgcatatatctccaactcttgctttttaatcccttggttaagaccgaggggatttgtcggaacagggtgactaacaacgtgaaagatgtgaccaaactggtggcaaacctcccgaaggactacatgattacactcaaatatgtgccgggcatggatgtcttgccaagccactgttggatctccgaaatggttgtccagttgtccgacagccttacggatctcctggataaatttagcaacattagcgaaggtctttctaattattccattatagataaactcgttaatattgtagatgacctcgtcgaatgtgtgaaggaaaattctagcaaggatttgaaaaaatcctttaagtcaccggaaccccgacttttcacccccgaagaatttttccgaatattcaacaggagcatagatgctttcaaagacttcgtagtggccagcgaaacaagtgactgcgtggtttcctatccttacgatgtcccggactatgctgctagcgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttga

SEQ ID NO: 46 - protein, 238 aa, Artificial sequence
  Synthetic
MKKTQTWILTCIYLQLLLFNPLVKTEGICRNRVTNNVKDVTKLVANLPKDYMITLKYVPGMDVLPSHCWISEMVVQLSDSLTDLLDKFSNISEGLSNYSIIDKLVNIVDDLVECVKENSSKDLKKSFKSPEPRLFTPEEFFRIFNRSIDAFKDFVVASETSDCVVSYPYDVPDYAASESKYGPPCPPCPAVGQDTQEVIVVPHSLPFKVVVISAILALVVLTIISLIILIMLWQKKPR

SEQ ID NO: 47 - DNA, 717 bp, Artificial sequence
  Synthetic
atgaagaagactcagacctggattctgacgtgcatatatctccaactcttgctttttaatcccttggttaagaccgaggggatttgtcggaacagggtgactaacaacgtgaaagatgtgaccaaactggtggcaaacctcccgaaggactacatgattacactcaaatatgtgccgggcatggatgtcttgccaagccactgttggatctccgaaatggttgtccagttgtccgacagccttacggatctcctggataaatttagcaacattagcgaaggtctttctaattattccattatagataaactcgttaatattgtagatgacctcgtcgaatgtgtgaaggaaaattctagcaaggatttgaaaaaatcctttaagtcaccggaaccccgacttttcacccccgaagaatttttccgaatattcaacaggagcatagatgctttcaaagacttcgtagtggccagcgaaacaagtgactgcgtggtttcctatccttacgatgtcccggactatgctgctagcgaaagcaagtatggtcctccctgccccccgtgcccagctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttga

SEQ ID NO: 48 - protein, 256 aa, Artificial sequence
  Synthetic
MTVLAPAWSPTTYLLLLLLLSSGLSGTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPYPYDVPDYAASESKYGPPCPPCPAVGQDTQEVIVVPHSLPFKVVVISAILALVVLTIISLIILIMLWQKKPR

SEQ ID NO: 49 - DNA, 771 bp, Artificial Sequence
  Synthetic
atgacagtgctggccccagcctggagtccaacaacctaccttctcttgctcttgcttctttccagtggcctgtcaggcacgcaagattgttcatttcaacattcacccatcagttcagactttgctgttaaaattagggagttgagcgattacctcctgcaagattatcctgtgactgttgcaagcaaccttcaggatgaagagctttgcggggggctctggcgcctcgtgttggctcagcggtggatggaacgcctcaaaacggtggcgggtagtaagatgcagggtctgttggagagagttaacacggagatccatttcgtaaccaagtgtgcatttcaaccgccaccctcttgccttagatttgtccaaaccaatatcagccgacttctccaagagacatctgaacagcttgttgccctgaaaccgtggattacaaggcaaaacttttcacgctgcttggagcttcaatgtcaacctgacagtagtacccttccgcctccttggtctcctagaccgcttgaagctacggctcctacggcaccacaaccctatccttacgatgtcccggactatgctgctagcgaaagcaagtatggtcctccctgccccccgtgcccagctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttga

SEQ ID NO: 50 - protein, 244 aa, Artificial sequence
  Synthetic
MTVLAPAWSPTTYLLLLLLLSSGLSGTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPYPYDVPDYAASAVGQDTQEVIVVPHSLPFKVVVISAILALVVLTIISLIILIMLWQKKPR

SEQ ID NO: 51 - DNA, 735 bp, Artificial sequence
  Synthetic
atgacagtgctggccccagcctggagtccaacaacctaccttctcttgctcttgcttctttccagtggcctgtcaggcacgcaagattgttcatttcaacattcacccatcagttcagactttgctgttaaaattagggagttgagcgattacctcctgcaagattatcctgtgactgttgcaagcaaccttcaggatgaagagctttgcggggggctctggcgcctcgtgttggctcagcggtggatggaacgcctcaaaacggtggcgggtagtaagatgcagggtctgttggagagagttaacacggagatccatttcgtaaccaagtgtgcatttcaaccgccaccctcttgccttagatttgtccaaaccaatatcagccgacttctccaagagacatctgaacagcttgttgccctgaaaccgtggattacaaggcaaaacttttcacgctgcttggagcttcaatgtcaacctgacagtagtacccttccgcctccttggtctcctagaccgcttgaagctacggctcctacggcaccacaaccctatccttacgatgtcccggactatgctgctagcgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttga

SEQ ID NO: 52 - protein, 6 aa, Artificial sequence
  Synthetic
  REPEAT (1)..(6)
  REPEAT (1)..(6): may be repeated 1 or more times
GAPGAS

SEQ ID NO: 53 - protein, 5 aa, Artificial sequence
  Synthetic
  REPEAT (1)..(5): may be repeated 1 or more times
GGGGS

(19) United States
(12) Patent Application Publication
(10) Pub. No.: US 2022/0340876 A1
(43) Pub. Date: Oct. 27, 2022

(54) VIRAL TARGETING OF HEMATOPOIETIC STEM CELLS

(71) Applicants: Massachusetts Institute of Technology, Cambridge, MA (US); The Regents of the University of California, Oakland, CA (US)

(72) Inventors: Michael Birnbaum, Somerville, MA (US); Connor Dobson, Somerville, MA (US); Stephanie Gaglione, Cambridge, MA (US); Kole Roybal, San Francisco, CA (US); Cassandra Burnett, San Francisco, CA (US)

(73) Assignees: Massachusetts Institute of Technology, Cambridge, MA (US); The Regents of the University of California, Oakland, CA (US)

(21) Appl. No.: 17/722,312

(22) Filed: Apr. 16, 2022

Related U.S. Application Data
(60) Provisional application No. 63/176,120, filed on Apr. 16, 2021.

(51) Int. Cl. C12N 5/0789; C12N 15/86; C12N 9/22; C12N 15/11; C12N 15/62

(52) U.S. Cl. CPC C12N 5/0647; C12N 15/86; C12N 9/22; C12N 15/11; C12N 15/625; C12N 2740/15022; C12N 2740/15023; C12N 2740/15042; C12N 2740/15044; C12N 2740/15045; C12N 2310/20; C12N 2800/80

(57) ABSTRACT

Disclosed herein are compositions of retroviruses and methods of using the same for gene delivery to a hematopoietic stem cell (HSC), wherein the retroviruses comprise a viral envelope protein comprising at least one mutation that diminishes its native function, a non-viral membrane-bound protein comprising a membrane-bound domain and an extracellular targeting domain.

### RELATED APPLICATIONS

This application claims priority under 35 U.S.C. § 119(e) to U.S. provisional patent application No. 63/176,120, filed Apr. 16, 2021, which is hereby incorporated by reference in its entirety.

### GOVERNMENT SUPPORT

This invention was made with government support under OD025751 awarded by the National Institutes of Health. The government has certain rights in the invention.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Apr. 11, 2022, is named M065670508US01-SEQ-GIC and is 105,170 bytes in size.

### BACKGROUND

Hematopoietic stem cells (HSCs) are progenitor cells that assist in the production of blood and cells of the adaptive immune system. Accordingly, HSCs are of central importance for the maintenance of immunity and normal bodily function. Since HSCs self-renew and divide to create billions of blood cells each day, HSC dysfunction is routinely problematic. Genetic diseases such as sickle cell anemia or severe combined immunodeficiency (SCID) can cause severe morbidities and mortality. Further, the proliferative ability of HSC-lineage cells and their descendants, combined with the ability of B and T cells to recombine their genomes, result in blood cancers such as leukemias and lymphomas.

Presently, the last-line treatment for cancer or genetic disorders (such as those related to HSC dysfunction) are bone marrow transplants. While transplants can be effective, there are several complicating factors. Finding an HLA-matched donor can be challenging and the lymphodepletion preconditioning regimen prior to the transplant can be toxic and poorly tolerated.

Thus, direct genetic alteration of HSCs would be extremely powerful to either correct genetic diseases or create a self-renewing source of engineered anti-tumor immune cells, such as CAR-T cells. However, this has been difficult to achieve. HSCs are both rare and heterogeneous, meaning that efficiently and selectively modifying their genomes is difficult.

### SUMMARY

Herein, the inventors have demonstrated that a combination of mutations to abolish native function (e.g., tropism) and overexpression of a second membrane protein allows for that second protein to function as the basis for viral entry into hematopoietic stem cells (HSCs). These discoveries, as described herein, enable new and innovative methodologies, for example, to screen cells that are notoriously challenging to screen for specific antigens and function, and to deliver nucleic acids to HSCs in an HSC-specific manner.

Some aspects of the disclosure provide a method of delivering one or more nucleic acids to a hematopoietic stem cell (HSC). In some embodiments, a method of delivering one or more nucleic acids to an HSC comprises: (i) providing a retrovirus comprising the one or more nucleic acids, a viral envelope protein comprising at least one mutation that diminishes its native function, and a non-viral membrane-bound protein comprising an extracellular targeting domain that binds to a protein on the surface of the HSC; and (ii) contacting the retrovirus with the HSC, thereby delivering the one or more nucleic acids to the HSC.

In some embodiments, the extracellular targeting domain is stem cell factor (SCF), FMS-like tyrosine kinase 3 ligand (FLT3L), or thrombopoietin (TPO). In some embodiments, the protein on the surface of the HSC is CD34, CD90, CD133, CD49f, CD201, c-Kit, FMS-like tyrosine kinase 3 (FLT3), or thrombopoietin receptor.

In some embodiments, at least one of the one or more nucleic acids encodes a gene of interest. In some embodiments, the gene of interest encodes a protein of interest. In some embodiments, the protein of interest is a gene editing protein. In some embodiments, the gene editing protein is a Cas endonuclease, a zinc finger nuclease, a transcription activator-like effector nuclease (TALEN), or a meganuclease. In some embodiments, the Cas endonuclease is a Cas9 endonuclease. In some embodiments, at least one of the one or more nucleic acids is a guide RNA.

In some embodiments, the retrovirus enters or infects the cell during (ii). In some embodiments, the retrovirus is a lentivirus. In some embodiments, the viral envelope protein is a VSV-G envelope protein or a cocal virus G protein. In some embodiments, at least one mutation of a VSV-G envelope protein is a mutation selected from the group consisting of H8, 141, K47, Y209, and R354. In some embodiments, the at least one mutation of the measles virus envelope protein is a mutation selected from the group consisting of Y481, R533, 5548, and F549. In some embodiments, the at least one mutation of the nipah virus envelope protein is a mutation selected from the group consisting of E501, W504, Q530, and E533. In some embodiments, the at least one mutation of the cocal virus G protein is a mutation selected from the group consisting of K64 and R371.

In some embodiments, a linker is positioned between the membrane-bound domain and the extracellular targeting domain. In some embodiments, the linker is a rigid linker. In some embodiments, the rigid linker comprises a PDGFR stalk or a CD8αstalk. In some embodiments, the linker is a flexible linker. In some embodiments, the flexible linker comprises an amino acid sequence comprising GAPGAS (SEQ ID NO: 5) or GGGGS (SEQ ID NO: 7). In some embodiments, the linker is an oligomerized linker. In some embodiments, the oligomerized linker comprises an IgG4 hinge or an amino acid sequence that can form a tetrameric coiled coil.

In some embodiments, the HSC is a murine HSC or a human HSC. In some embodiments, the one or more nucleic acids encode a chimeric antigen receptor.

Some aspects of the disclosure provide a method of gene editing in a hematopoietic stem cell (HSC). In some embodiments, a method of gene editing in an HSC comprises (i) providing a retrovirus comprising one or more nucleic acids encoding a gene editing composition, a viral envelope protein comprising at least one mutation that diminishes its native function, and a non-viral membrane-bound protein comprising an extracellular targeting domain that binds to a protein on the surface of the HSC; and (ii) contacting the retrovirus with the HSC such that the one or more nucleic acids encoding a gene editing composition are delivered to the HSC, wherein the gene editing composition specifically targets a section of the chromosomal DNA of the HSC to cause a genetic modification.

In some embodiments, the extracellular targeting domain is stem cell factor (SCF), FMS-like tyrosine kinase 3 ligand (FLT3L), or thrombopoietin (TPO). In some embodiments, the protein on the surface of the HSC is CD34, CD90, CD133, CD49f, CD201, c-Kit, FMS-like tyrosine kinase 3 (FLT3), or thrombopoietin receptor.

In some embodiments, the gene editing composition comprises one of the one or more nucleic acids, wherein the one or more nucleic acids encode a gene editing protein and/or a guide RNA. In some embodiments, the gene editing protein is a Cas endonuclease, a zinc finger nuclease, a transcription activator-like effector nuclease (TALEN), or a meganuclease. In some embodiments, the Cas endonuclease is a Cas9 endonuclease.

In some embodiments, the retrovirus enters or infects the cell during (ii). In some embodiments, the retrovirus is a lentivirus. In some embodiments, the viral envelope protein is a VSV-G envelope protein or a cocal virus G protein. In some embodiments, at least one mutation of a VSV-G envelope protein is a mutation selected from the group consisting of H8, 141, K47, Y209, and R354. In some embodiments, the at least one mutation of the measles virus envelope protein is a mutation selected from the group consisting of Y481, R533, 5548, and F549. In some embodiments, the at least one mutation of the nipah virus envelope protein is a mutation selected from the group consisting of E501, W504, Q530, and E533. In some embodiments, the at least one mutation of the cocal virus G protein is a mutation selected from the group consisting of K64 and R371.

In some embodiments, a linker is positioned between the membrane-bound domain and the extracellular targeting domain. In some embodiments, the linker is a rigid linker. In some embodiments, the rigid linker comprises a PDGFR stalk or a CD8αstalk. In some embodiments, the linker is a flexible linker. In some embodiments, the flexible linker comprises an amino acid sequence comprising GAPGAS (SEQ ID NO: 5) or GGGGS (SEQ ID NO: 7). In some embodiments, the linker is an oligomerized linker. In some embodiments, the oligomerized linker comprises an IgG4 hinge or an amino acid sequence that can form a tetrameric coiled coil.

In some embodiments, the HSC is a murine HSC or a human HSC. In some embodiments, the method further comprises delivering one or more nucleic acids encoding a chimeric antigen receptor to the HSC.

### BRIEF DESCRIPTION OF THE DRAWINGS

FIG. 1 depicts the expression of a HA tag on HEK cells for SCFa PStalk and IgG4Hinge constructs.

FIG. 2 depicts the expression of a HA tag on HEK cells for S4-3a PStalk and IgG4Hinge constructs.

FIG. 3 depicts unconcentrated virus expression after mixing with MC9 cKIT-expressing cells. 1:1 ratio of MC9 Cells:virus.

FIG. 4 depicts unconcentrated virus expression after mixing with MC9 cKIT-expressing cells. 2:1 ratio of MC9 Cells:virus.

FIG. 5 depicts unconcentrated virus expression after mixing with MC9 cKIT-expressing cells. 4:1 ratio of MC9 Cells:virus.

FIG. 6 depicts viral entry of mSCFa-IgG4Hinge-VSVd at 5 uL, 2.5 uL 1.25 uL and 0.625 uL (left-right), in MC9 cells, in the presence or absence of polybrene.

FIG. 7 depicts viral entry of mS4-3a-IgG4Hinge-VSVd at 5 uL, 2.5 uL 1.25 uL and 0.625 uL (left-right), in MC9 cells, in the presence or absence of polybrene.

FIG. 8 depicts expression of off-target virus control, mFLT3L-IgG4Hinge-VSVd, and VSVd virus alone in MC9 cells.

FIG. 9 depicts expression of virus constructs (5 uL, with polybrene) in VhCm non-cKIT-expressing cells.

FIG. 10 depicts a schematic of the experimental design to test efficiency and specificity of SCF-WT, SCF-Mutant, and FLT3 specific lentivirus in primary mouse bone marrow cells.

FIG. 11 depicts the efficiency of SCF virus variants with and without SCF in MC9 cells (murine mast cell line).

FIGS. 12A-12B depict the efficiency of SCF-virus variants measured by GFP+in sorted LSK (Lin-, Sca-1+, cKIT+), cKIT enriched,lineage depleted, and WBM (whole bone marrow) cells.

FIGS. 13A-13B depict the efficiency of SCF-virus variants measured by GFP+in cKIT enriched cells, with and without SCF.

FIG. 14 depicts the specificity of SCF-virus variants measured by GFP+in sorted LSK (Lin-, Sca-1+, cKIT+), lineage depleted, and WBM cells.

FIGS. 15A-15C depict the efficiency of FLT3-virus variants measured by GFP+in HSC-FLT3 sorted, cKIT enriched, lineage depleted, and WBM cells.

FIGS. 16A-16B depict the efficiency of FLT3-virus variants measured by GFP+in cKIT enriched cells, with and without FLT3.

FIG. 17 depicts the specificity of FLT3-virus variants measured by GFP+in HSC −FLT3 sorted, lineage depleted, and WBM cells.

FIGS. 18A-18H provide example mouse constructs of the disclosure.

FIGS. 19A-19D provide example human constructs of the disclosure.

### DETAILED DESCRIPTION OF THE INVENTION

Herein are provided new and innovative methods, for example, to deliver nucleic acids (e.g., for gene replacement or gene editing) to target cells in a target-specific manner. In some embodiments, described herein are systems that enable, for example, nucleic acid delivery (e.g., nucleic acids encoding a gene) to hematopoietic stem cells (HSCs). In some embodiments, described herein are retrovirus-based systems that repurpose viral tropism as a method of selecting for molecular interactions and replace the binding functions of wild-type virus surface proteins with those of protein variants of interest, for example, by encoding these protein variants on the corresponding transfer plasmid used to make the virus, thereby ensuring that the resulting virus displays the protein variant on its surface and packaging the corresponding genetic sequence. As such, when the virus enters a target cell (e.g., bearing a receptor that binds the displayed extracellular targeting domain of the protein variant), cell entry results in integration of the genetic sequence of the displayed protein into the genome of the target cell.

‘VSVdead’ affinity-ablated viral fusogen can be co-expressed with constructs containing murine stem cell factor (mSCF) on the surface of a lentivirus. Presented herein are constructs based upon mSCF: monomeric versions in which mSCF is tethered to the PDGFR stalk and transmembrane protein, as well as pre-dimerized using an Fc hinge region. The ‘wild type’ mSCF with endogenous affinity for the cKIT receptor was also used, as well as S4-3a, an affinity matured version of SCF which could show more efficient viral entry (as previously described in Ho C C et al., Cell 2017).

It is demonstrated herein that (a) these proteins all express on the surface of an HEK viral packaging cell line, (b) the SCF proteins enable selective viral entry into MC9 cells (which are not HSCs, but a Mast Cell-based immortalized cell line that is cKIT+), and (c) do not enter cKIT-cells. This works with comparable efficiency to other B and T cell targeting approaches. Additionally, a construct to target via FLT3L, another ligand that may show efficacy, was created and was shown to express. These viruses were tested (a) in vitro in murine HSCs and (b) delivered in vivo.

The methods presented herein enable a broad range of applications in cell and gene therapy, dramatically increasing the scope of what can be accomplished while also reducing cost and could potentially be impactful to a broad range of diseases.

Retroviruses Described herein are retroviruses comprising a viral envelope protein comprising at least one mutation that diminishes its native function, a non-viral membrane-bound protein comprising a membrane-bound domain and an extracellular targeting domain, and a nucleic acid encoding a reporter. In some embodiments, a retrovirus comprises a viral envelope protein comprising at least one mutation that diminishes its native function and a non-viral membrane-bound protein comprising a membrane-bound domain and an extracellular targeting domain.

The retrovirus disclosed herein comprise one or more elements derived from a retroviral genome (naturally-occurring or modified) of a suitable species. Retroviruses include 7 families: alpharetrovirus (Avian leucosis virus), betaretrovirus (Mouse mammary tumor virus), gammaretrovirus (Murine leukemia virus), deltaretrovirus (Bovine leukemia virus), epsilonretrovirus (Walleye dermal sarcoma virus), lentivirus (Human immunodeficiency virus 1), and spumavirus (Human spumavirus). Six additional examples of retroviruses are provided in U.S. Pat. No. 7,901,671.

In some embodiments, a retrovirus is a lentivirus. Lentivirus is a genus of retroviruses that typically gives rise to slowly developing diseases due to their ability to incorporate into a host genome. Modified lentiviral genomes are useful as viral vectors for the delivery of a nucleic acids to a host cell. Host cells can be transfected with lentiviral vectors, and optionally additional vectors for expressing lentiviral packaging proteins (e.g., VSV-G, Rev, and Gag/Pol) to produce lentiviral particles in the culture medium.

Retrovirus and lentivirus constructs are well known in the art and any suitable retrovirus can be used to construct the retrovirus (or a plurality or library of retroviruses) as described herein. Non-limiting examples of retrovirus constructs include lentiviral vectors, human immunodeficiency viral (HIV) vector, avian leucosis viral (ALV) vector, murine leukemia viral (MLV) vector, murine mammary tumor viral (MMTV) vector, murine stem cell virus, and human T cell leukemia viral (HTLV) vector. These retrovirus constructs comprise proviral sequences from the corresponding retrovirus.

The retrovirus described herein may comprise the viral elements such as those described herein from one or more suitable retroviruses, which are RNA viruses with a single strand positive-sense RNA molecule. Retroviruses comprise a reverse transcriptase enzyme and an integrase enzyme. Upon entry into a target cell, retroviruses utilize their reverse transcriptase to transcribe their RNA molecule into a DNA molecule. Subsequently, the integrase enzyme is used to integrate the DNA molecule into the host cell genome. Upon integration into the host cell genome, the sequence from the retrovirus is referred to as a provirus (e.g., proviral sequence or provirus sequence). The retroviral vectors described herein may further comprise additional functional elements as known in the art to address safety concerns and/or to improve vector functions, such as packaging efficiency and/or viral titer. Additional information may be found in US20150316511 and WO2015/117027, the relevant disclosures of each of which are herein incorporated by reference for the purpose and subject matter referenced herein. Additional information for lentiviruses can be found in, e.g., WO2019/056015, the relevant disclosures of which are incorporated by reference herein for this particular purpose.

Viral envelope protein The retroviruses described herein comprise a viral envelope protein comprising at least one mutation that diminishes its native function (e.g., wild-type function of a non-mutated viral envelope protein). In some embodiments, a viral envelope protein is any viral envelope protein of any retrovirus (e.g., lentivirus). A viral envelope protein may be a VSV-G envelope protein, a measles virus envelope protein, a nipah virus envelope protein, or a cocal virus G protein. In some embodiments, a wild-type or non-mutated VSV-G envelope protein has the amino acid sequence of SEQ ID NO: 12 (with leader sequence) or SEQ ID NO: 13 (without leader sequence). In some embodiments, a wild-type or non-mutated measles virus envelope protein has the amino acid sequence of SEQ ID NO: 19 (with leader sequence). In some embodiments, a wild-type or non-mutated cocal virus G protein has the amino acid sequence of SEQ ID NO: 24. In some embodiments, the native function that is diminished by a mutation of a viral envelope protein is viral tropism (e.g., ability to infect cells, bind to cells, etc.).

In some embodiments, a viral envelope protein comprising at least one mutation that diminishes its native function is a mutated VSV-G envelope protein. In some embodiments, a viral envelope protein comprising at least one mutation that diminishes its native function is a mutated measles virus envelope protein. In some embodiments, a viral envelope protein comprising at least one mutation that diminishes its native function is a mutated nipah virus envelope protein. In some embodiments, a viral envelope protein comprising at least one mutation that diminishes its native function is a mutated cocal virus G protein.

In some embodiments, a mutated VSV-G envelope protein comprises a mutation at H8, 141, K47, Y209, and/or R354. The position for an amino acid substitution in the mutated VSV-G envelope protein is identified in reference to the wildtype VSV-G envelope protein without the leader sequence, for example as provided in SEQ ID NO: 13. In some embodiments, a mutated VSV-G envelope protein comprises a HBA, I41L, K47A, K47Q, Y209A, R354A, and/or R354Q mutation. In some embodiments, a mutated VSV-env protein comprises an I41L, K47Q, and R354A mutation, such as a mutated VSV-env protein set forth in SEQ ID NO: 16. In some embodiments, a mutated VSV-env protein comprises a K47Q and R354A mutation, such as a mutated VSV-env protein set forth in SEQ ID NO: 17. In some embodiments, a mutated VSV-G envelope protein is as described in Nikolic et al., “Structural basis for the recognition of LDL-receptor family members by VSV glycoprotein.” Nature Comm., 2018, 9:1029, the relevant disclosures of which are incorporated by reference herein for this particular purpose.

In some embodiments, a mutated measles virus envelope protein comprises a mutation at Y481, R533, 5548, and/or F549. In some embodiments, a mutated measles virus envelope protein comprises a Y481A, R533A, S548L, and/or F549S mutation. In some embodiments, a mutated measles virus envelope protein comprises the mutated measles virus envelope protein set forth in SEQ ID NO: 21.

In some embodiments, a mutated Nipah virus envelope protein comprises a mutation at E501, W504, Q530, and/or E533. In some embodiments, a mutated measles virus envelope protein comprises a E501A, W504A, Q530A, and/or E533A mutation. In some embodiments, a mutated Nipah virus envelope protein comprises the mutated Nipah virus envelope protein set forth in SEQ ID NO: 23.

In some embodiments, a mutated cocal virus G protein comprises a mutation at K64 and/or R371. In some embodiments, a mutated cocal virus G protein comprises a mutation at K64Q and/or R371A. The position for an amino acid substitution in the mutated cocal virus G protein is identified in reference to the wildtype cocal virus G protein, for example as provided in SEQ ID NO: 24. In some embodiments, a mutated cocal virus G protein comprises a K64Q and R371A mutation, such as the mutated cocal virus G protein set forth in SEQ ID NO: 26.

In some embodiments, the mutated envelope protein is derived from any other enveloped virus including but not limited to baculovirus, herpes simplex virus (HSV), cytomegalovirus (CMV), lymphocytic choriomeningitis virus (LCMV), Epstein-Barr virus (EBV), vaccinia virus, Hepatitis A, B, or C virus, vaccinia virus, alphavirus, dengue virus, yellow fever virus, Zika virus, influenza virus, hantavirus, Ebola virus, rabies virus, human immunodeficiency virus (HIV), coronavirus, and other members of rhabdoviridae.

In some embodiments, a viral envelope protein comprising at least one mutation comprises 1, 2, 3, 4, 5, 6, 7, 8, 9, 10, or more mutations. In some embodiments, a viral envelope protein comprising at least one mutation comprises a nucleotide sequence and/or amino acid sequence that is at least 50%, 60%, 70%, 80%, 90%, 95%, or 97% identical to a wild-type viral envelope protein. In some embodiments, a viral envelope protein comprising at least one mutation that diminishes its native function retains less than 95%, 90%, 80%, 70%, 60%, 50%, 40%, 30%, 20%, or 10% of the function of a wild-type viral envelope protein. In some embodiments, a viral envelope protein comprising at least one mutation lacks all of its native function. In some embodiments, a retrovirus comprising a viral envelope protein comprising at least one mutation that diminishes its native function comprises less than 95%, 90%, 80%, 70%, 60%, 50%, 40%, 30%, 20%, or 10% of the cellular infectivity of a retrovirus comprising a wild-type viral envelope protein.

Non-viral membrane-bound protein The retroviruses described herein comprise a non-viral membrane-bound protein. A non-viral membrane-bound protein may comprise a membrane-bound domain and an extracellular targeting domain that binds to a protein on the surface of a hematopoietic stem cell (HSC). In some embodiments, a non-viral membrane-bound protein is a chimeric protein comprising sequences from at least two different proteins. In some embodiments, a non-viral membrane-bound protein is a full-length or truncated protein comprising sequence from a single protein.

A membrane-bound domain is a protein or peptide that has an amino acid sequence that enables the protein or peptide to be fully or partially embedded or associated with the membrane (e.g., envelope) of the retrovirus. In some embodiments, a membrane-bound domain enables presentation and delivery of the extracellular targeting domain to the extracellular environment. In some embodiments, a membrane-bound domain comprises an intracellular domain, a transmembrane domain, and/or an extracellular domain. In some embodiments, a membrane-bound domain comprises an intracellular domain and a transmembrane domain. In some embodiments, the membrane-bound domain comprises a Major Histocompatibility Complex (MHC) protein or fragment thereof. A MHC protein may be a Class I or Class II MHC protein.

In some embodiments, a membrane-bound domain comprises 10-50, 10-100, 25-100, 50-200, 50-150, 100-500, 100-250, 250-500, or any reasonable number of total amino acids.

In some embodiments, a retrovirus present in a library of retroviruses comprises the same membrane-bound domain as some or all of the other retroviruses in the library. In some embodiments, each retrovirus present in a library of retroviruses comprises a different membrane-bound domain relative to some or all of the other retroviruses in the library.

In some embodiments, an extracellular targeting domain is any protein or peptide that has an amino acid sequence and is a binding partner for a target molecule or ligand (e.g., a cognate protein) on a surface of a hematopoietic stem cell (HSC). When present in the extracellular environment beyond the interior of the retrovirus, an extracellular targeting domain is capable of binding to an HSC. In some embodiments, an extracellular targeting domain binds or targets to a cognate protein or ligand (e.g., a protein receptor present on a target HSC) that is present on the cellular surface of an HSC or a subset of a population of HSCs. In some embodiments, an extracellular targeting domain binds to a cognate protein or ligand that is present on the cell surface of a single HSC or a subset of a population of HSCs. In some embodiments, a binding interaction between an extracellular targeting domain of a retrovirus and a cognate protein or ligand of a cell enables the retrovirus to enter the HSC.

In some embodiments, an extracellular targeting domain comprises 10-50, 10-100, 25-100, 50-200, 50-150, 100-500, 100-250, 250-500, or any reasonable number of total amino acids. In some embodiments, an extracellular targeting domain comprises at least 5, at least 10, at least 15, at least 20, or at least 50 amino acids.

In some embodiments, an extracellular targeting domain is a protein, an antibody or peptide. In some embodiments, an antibody is a full-length antibody, an antibody fragment, a nanobody, or a single chain antibody (scFv). In some embodiments, an extracellular targeting domain is an antibody that binds to a cognate protein of a target cell. In some embodiments, an extracellular targeting domain is an antibody that binds to an HSC antigen. In some embodiments, an extracellular targeting domain is a protein or peptide that binds to a receptor (e.g., a receptor that is present on the surface of a target cell).

In some embodiments, an extracellular targeting domain is stem cell factor (SCF), FMS-like tyrosine kinase 3 ligand (FLT3L), or thrombopoietin (TPO). In some embodiments, an extracellular targeting domain comprises the amino acid sequence set forth in any one of SEQ ID NOs. 54-59.

In some embodiments, an extracellular targeting domain binds to a protein on the surface of the HSC selected from the group consisting of: CD34, CD90, CD133, CD49f, CD201, c-Kit, FMS-like tyrosine kinase 3 (FLT3), and thrombopoietin receptor.

In some embodiments, an extracellular targeting domain is a protein or peptide that binds to a cytokine receptor (e.g., interleukin-13 (IL-13) receptor). In some embodiments, an extracellular targeting domain is a cytokine (e.g., IL-2, IL-6, IL-12, IL-13). In some embodiments, an extracellular targeting domain is a chemokine ligand (e.g. CXCL9, CXCL10, CXCL 11, etc.). In some embodiments, an extracellular targeting domain is a cellular receptor, including cytokine receptors (e.g. IL-13Rα1, IL-13Rα2, IL-2 receptors, common gamma chain), GPCRs (including chemokine receptors such as CSCR3, CXCR4, etc.), and integrins. In some embodiments, an extracellular targeting domain is a peptide that is displayed by a MHC protein. In some embodiments, non-viral membrane-bound protein comprises a membrane-bound domain comprising a MHC protein or fragment and an extracellular targeting domain comprising a peptide that is displayed by a MHC protein.

In some embodiments, an extracellular targeting domain binds to a target cell or cell surface molecule with a binding affinity of 10⁻⁹ to 10⁻⁸M, 10⁻⁸ to 10⁻⁷M, 10⁻⁷ to 10⁻⁶M, 10⁻⁶ to 10⁻⁵M, 10⁻⁵ to 10⁴M, 10⁻⁴ to 10⁻³M, or 10⁻³ to 10⁻²M. In some embodiments, an extracellular targeting domain binds to a cognate protein or ligand of a target cell with a binding affinity of 10⁻⁹_{to 10}⁻⁸M, 10⁻⁸_{to 10}⁻⁷M, 10⁻⁷ to 10⁻⁶M, 10⁻⁶ to 10⁻⁵M, 10⁻⁵ to 10⁴M, 10⁴ to 10⁻³M, or 10⁻³ to 10⁻²M. In some embodiments, the binding affinity between an extracellular targeting domain and a cognate protein or ligand is in the picomolar to nanomolar range (e.g., between about 10⁻¹² and about 10⁻⁹M). In some embodiments, the binding affinity between an extracellular targeting domain and a cognate protein or ligand is in the nanomolar to micromolar range (e.g., between about 10⁻⁹ and about 10⁻⁶M). In some embodiments, the binding affinity between an extracellular targeting domain and a cognate protein or ligand is in the micromolar to millimolar range (e.g., between about 10⁻⁶ and about 10⁻³M). In some embodiments, the binding affinity between an extracellular targeting domain and a cognate protein or ligand is in the picomolar to micromolar range (e.g., between about 10⁻¹² and about 10⁻⁶M). In some embodiments, the binding affinity between an extracellular targeting domain and a cognate protein or ligand is in the nanomolar to millimolar range (e.g., between about 10⁻⁹ and about 10⁻³M).

As used herein, the term antibody generally refers to a protein that includes at least one immunoglobulin variable domain or immunoglobulin variable domain sequence. For example, an antibody can include a heavy (H) chain variable region (abbreviated herein as V_{H}), and/or a light (L) chain variable region (abbreviated herein as V_{L}). In another example, an antibody includes two heavy (H) chain variable regions and/or two light (L) chain variable regions. An antibody can have the structural features of IgA, IgG, IgE, IgD, IgM (as well as subtypes thereof). The V_{H} and V_{L} regions can be further subdivided into regions of hypervariability, termed “complementarity determining regions” (“CDR”), interspersed with regions that are more conserved, termed “framework regions” (“FR”). Each V_{H} and/or V_{L} is typically composed of three CDRs and four FRs, arranged from amino-terminus to carboxy-terminus in the following order: FR1, CDR1, FR2, CDR2, FR3, CDR3, FR4. The V_{H} or V_{L} chain of the antibody can further include a heavy or light chain constant region, to thereby form a heavy or light immunoglobulin chain, respectively. In some embodiments, the antibody is a tetramer of two heavy immunoglobulin chains and two light immunoglobulin chains, wherein the heavy and light immunoglobulin chains are inter-connected by, e.g., disulfide bonds. In IgGs, the heavy chain constant region includes three immunoglobulin domains, CH1, CH2 and CH3.

In some embodiments, a retrovirus present in a library of retroviruses comprises the same extracellular targeting domain as some or all of the other retroviruses in the library. In some embodiments, each retrovirus present in a library of retroviruses comprises a different extracellular targeting domain relative to some or all of the other retroviruses in the library.

In some embodiments, a non-viral membrane-bound protein further comprises a signal sequence (also referred to as a signal peptide of localization sequence). In some embodiments, the signal sequence is at the N- or C-terminal ends of the non-viral membrane-bound protein. A signal sequence functions to translocate the non-viral membrane-bound protein to the membrane (or envelope) of the retrovirus. In some embodiments, a signal sequence is 5-10, 5-15, 10-20, 15-20, 15-30, 20-30, or 25-30 amino acids. In some embodiments, the signal sequence is an Ig Kappa leader sequence (e.g., a murine Ig Kappa leader sequence comprising: METDTLLLWVLLLWVPGSTG (SEQ ID NO: 1)) or a B2M signal peptide sequence (e.g., a B2M signal peptide sequence comprising: MSRSVALAVLALLSLSGLEA (SEQ ID NO: 2)). In some embodiments, a retrovirus present in a library of retroviruses comprises the same signal sequence as some or all of the other retroviruses in the library. In some embodiments, each retrovirus present in a library of retroviruses comprises a different signal sequence relative to some or all of the other retroviruses in the library.

In some embodiments, a nucleic acid encoding a non-viral membrane-bound protein further comprises an internal ribosome entry site (IRES). An IRES is an RNA sequence that allows for initiation of translation during protein synthesis. In some embodiments, the IRES is located at or near the C-terminal end. In some embodiments, the IRES is located C-terminal relative to the membrane-bound domain and the extracellular targeting domain. In some embodiments, the IRES is a viral IRES. In some embodiments, the IRES is an IRES that is native to the retrovirus. In some embodiments, the IRES is a sequence derived from encephalomyocarditis virus (EMCV). In some embodiments, a retrovirus present in a library of retroviruses comprises the same IRES as some or all of the other retroviruses in the library. In some embodiments, each retrovirus present in a library of retroviruses comprises a different IRES relative to some or all of the other retroviruses in the library.

In some embodiments, a non-viral membrane-bound protein further comprises a linker positioned between the membrane-bound domain and the extracellular targeting domain. A linker is an amino acid linker and may be a rigid linker, a flexible linker, or an oligomerized linker. A rigid linker is an amino acid sequence that lacks flexibility (e.g., may comprise at least one proline). In some embodiments, a rigid linker comprises a platelet-derived growth factor receptor (PDGFR) stalk or a CD8αstalk. In some embodiments, a PDGFR stalk comprises an amino acid sequence comprising AVGQDTQEVIVVPHSLPFK (SEQ ID NO: 3). In some embodiments, a PDGFR stalk comprises an amino acid sequence comprising

(SEQ ID NO: 4)
ASAKPTTTPAPRPPTPAPTIASQPLSLRPEAARPAAGGAVHTRGLDFAK

A flexible linker is an amino acid sequence that has many degrees of freedom (e.g., may comprise a plurality of amino acids with small side chains, e.g., glycine or alanine). In some embodiments, a flexible linker comprises an amino acid sequence comprising GAPGAS (SEQ ID NO: 5). In some embodiments, a flexible linker comprises an amino acid sequence consisting of GAPGSGGGGSGGGGSAS (SEQ ID NO: 6). In some embodiments, a flexible linker comprises an amino acid sequence comprising GGGGS (SEQ ID NO: 7). In some embodiments, a flexible linker comprises an amino acid sequence comprising (GAPGAS)_{N} (SEQ ID NO: 52) or (G4S)N(SEQ ID NO: 53), wherein N is 1, 2, 3, 4, 5, 6, 7, 8, 9, 10, or more. An oligomerized linker is an amino acid that can oligomerize to another related amino acid. In some embodiments, an oligomerized linker is an amino acid sequence that can form a dimer, trimer, or tetramer. In some embodiments, an oligomerized linker comprises an IgG4 hinge domain (e.g., ASESKYGPPCPPCPAVGQDTQEVIVVPHSLPFK (SEQ ID NO: 8)). In some embodiments, an oligomerized linker comprises an amino acid sequence that can form a tetrameric coiled coil (e.g., ASGGGGSGELAAIKQELAAIKKELAAIKWELAAIKQGAG (SEQ ID NO: 9)). In some embodiments, an oligomerized linker comprises an amino acid sequence that can form a dimeric coiled coil (e.g., ASESKYGPPCPPCP (SEQ ID NO: 10)).

In some embodiments, a non-viral membrane-bound protein comprises SCF or a truncated version thereof, a PGDFR stalk, and a PGDFRb transmembrane domain. In some embodiments, a non-viral membrane-bound protein comprises S4-3a or a truncated version thereof, a PGDFR stalk, and a PGDFRb transmembrane domain. In some embodiments, a non-viral membrane-bound protein comprises FLT3L, a PGDFR stalk, and a PGDFRb transmembrane domain. In some embodiments, a non-viral membrane-bound protein comprises TPO or a truncated version thereof, a PGDFR stalk, and a PGDFRb transmembrane domain.

In some embodiments, a non-viral membrane-bound protein comprises SCF or a truncated version thereof, a IgG4 hinge, and a PGDFRb transmembrane domain. In some embodiments, a non-viral membrane-bound protein comprises S4-3a or a truncated version thereof, a IgG4 hinge, and a PGDFRb transmembrane domain. In some embodiments, a non-viral membrane-bound protein comprises FLT3L, a IgG4 hinge, and a PGDFRb transmembrane domain. In some embodiments, a non-viral membrane-bound protein comprises TPO or a truncated version thereof, a IgG4 hinge, and a PGDFRb transmembrane domain.

In some embodiments, a non-viral membrane-bound protein comprises the amino acid sequence set forth in any one of SEQ ID NOs. 28, 29, 32, 34, 36, 38, 40, 42, 44, 46, 48, or 50.

Methods of delivering a nucleic acid to a hematopoietic stem cell (HSC) Described herein are methods of delivering a nucleic acid to an HSC, comprising (i) providing a retrovirus, as described herein, comprising the nucleic acid, a viral envelope protein comprising at least one mutation that diminishes its native function, and a non-viral membrane-bound protein comprising an extracellular targeting domain that is capable of binding to a cognate ligand of the cell; and (ii) contacting the retrovirus with the cell such that the retrovirus enters or infects the cell. In some embodiments, the nucleic acid encodes an mRNA molecule. In some embodiments, the mRNA is a gene of interest. In some embodiments, the nucleic acid encodes a double-stranded RNA, an antisense RNA, a microRNA, or any other RNA molecule. In some embodiments, the gene of interest encodes a protein. In some embodiments, the gene of interest encodes a therapeutic protein (e.g., a protein to compensate for a diseased condition in a subject).

In some embodiments, the nucleic acid encodes a chimeric antigen receptor. A chimeric antigen receptor, in some embodiments, comprises an extracellular domain comprising an antigen binding domain (e.g., an antibody, such as an scFv), a transmembrane domain, and a cytoplasmic domain. In some embodiments, the extracellular domain specifically binds a tumor antigen. In some embodiments, the tumor antigen is any one of CD19, BCMA, alpha folate receptor, 5T4, Ab integrin, B7-H3, B7-H6, CAIX, CD20, CD22, CD23, CD30, CD33, CD38, CD44, CD44v6, CD44v7/8, CD52, CD70, CD79a, CD79b, CD80, CD123, CD138, CD171, CEA, CSPG4, EGFR, ErbB2 (HER2), EGFRvIII, EGP2, EGP40, EpCAM, FAP, fetal AchR, FLT3, Fra, GD2, GD3, Glypican-3 (GPC3), HLA-A1+MAGE1, HLA-A2+MAGE1, HLA-A3 +MAGE1, HLA-A1+NY-ESO-1, HLA-A2+NY-ESO-1, HLA-A3+NY-ESO-1, HLADR, IL-11Ralpha, IL-13 Ralpha2, Lambda, Lewis-Y, Kappa, mesothelin, Muc1, Muc16, NCAM, NKG2d ligands, NY-ESO-1, PRAME, PSCA, PSMA, ROR1, SSX, Survivin, TAG72, TEMs, VEGFR2, BAFF-R, Claudin18.2, CD86, FcRL5, GPRC5, and TACI. In some embodiments, the extracellular domain of a chimeric antigen receptor includes an antigen binding domain and at least one of a signal peptide and/or an extracellular spacer domain (e.g., hinge domain). In some embodiments, the signal peptide enhances antigen specificity of the chimeric antigen receptor. In some embodiments, the extracellular spacer domain is located between the antigen binding domain and the transmembrane domain of the chimeric antigen receptor. In some embodiments, a hinge domain is a hinge domain from IgG1, IgG2, IgG3, IgG4, IgA, IgD, CD8a, CD4, CD28 or CD7. In some embodiments, the transmembrane domain is a hydrophobic alpha helix that spans cellular membrane to provide stability to the chimeric antigen receptor. In some embodiments, a transmembrane domain is a transmembrane domain of CD28, CD2, CD4, CD8a, CD5, CD3ϵ, CD3 δ, CD3ζ, CD9, CD16, CD22, CD25, CD27, CD33, CD37, CD40, CD45, CD64, CD79A, CD79B, CD80, CD86, CD95 (Fas), CD134 (OX40), CD137 (4-1BB), CD150 (SLAMF1), CD152 (CTLA4), CD154 (CD40L), CD200R, CD223 (LAG3), CD270 (HVEM), CD272 (BTLA), CD273 (PD-L2), CD274 (PD-L1), CD278 (ICOS), CD279 (PD-1), CD300, CD357 (GITR), A2aR, DAP10, FcRα, FcRβ, FcRγ, Fyn, GALS, KIR, Lck, LAT, LRP, NKG2D, NOTCH1, NOTCH2, NOTCH3, NOTCH4, PTCH2, ROR2, Ryk, Slp76, SIRPα, pTα, TCRα, TCRβ, TIM3, TRIM, LPAS, and Zap70. In some embodiments, the cytoplasmic domain of the chimeric antigen receptor is a protein domain that, following antigen recognition, causes signal transduction within the cell. In some embodiments, the cytoplasmic domain comprises an ITAM containing signaling domain. In some embodiments, an ITAM containing signaling domain is an intracellular signaling domain of any one of CD3γ, CD3 δ, CD3ϵ, CD3ζ, CD5, CD22, CD79a, CD278 (ICOS), DAP10, DAP12, FcRγ, and CD66d. In some embodiments, the cytoplasmic domain further comprises one or more costimulatory signaling domain(s). In some embodiments, a costimulatory signaling domain is an intracellular signaling domain of any one of CD27, CD28, CD40L, GITR, NKG2C, CARD1, CD2, CD7, CD27, CD30, CD40, CD54 (ICAM), CD83, CD134 (OX-40), CD137 (4-1BB), CD150 (SLAMF1), CD152 (CTLA4), CD223 (LAG3), CD226, CD270 (HVEM), CD273 (PD-L2), CD274 (PD-L1), CD278 (ICOS), DAP10, LAT, LFA-1, LIGHT, NKG2C, NKD2C, SLP76, TRIM, and ZAP70.

In some embodiments, the nucleic acid encodes a gene editing protein. A gene editing protein may be a Cas endonuclease, a Cpf1 endonuclease, a zinc finger nuclease, a transcription activator-like effector nuclease (TALEN), or a meganuclease.

A Cas endonuclease may be a Cas9 endonuclease, a dead Cas endonuclease (dCas, e.g., dCas9) In some embodiments, a Cas endonuclease is from Streptococcus pyogenes. A Cas endonuclease may be a wild-type Cas endonuclease or a modified or mutant versions of Cas endonuclease.

In some embodiments, the nucleic acid is a guide RNA. In some embodiments, a guide RNA is 20-200, 20-100, 50-200, 50-150, or about 100 nucleotides in length. In some embodiments, a guide RNA is a single-molecule guide RNA. In some embodiments, a guide RNA comprises a spacer sequence that binds to a target gene sequence. In some embodiments, a spacer sequence is 10, 11, 12, 13, 14, 15, 16, 17, 18, 19, 20, 21, 22, 23, 24, or 25 nucleotides in length.

In some embodiments, the nucleic acid is delivered to the cell when the retrovirus enters or infects the cell during step (ii). In some embodiments, the methods of delivering a nucleic acid described herein do not require a transfection agent (e.g., a lipophilic transfection agent such as Lipofectin).

Methods of gene editing

Described herein are methods of gene editing in a target cell (e.g., a hematopoietic stem cell (HSC)) comprising (i) providing a retrovirus comprising one or more nucleic acids encoding a gene editing composition, a viral envelope protein comprising at least one mutation that diminishes its native function, and a non-viral membrane-bound protein comprising an extracellular targeting domain that binds to a protein on the surface of the target cell; and (ii) contacting the retrovirus with the target cell such that the one or more nucleic acids encoding a gene editing composition are delivered to the target cell, wherein the gene editing composition specifically targets a section of the chromosomal DNA of the target cell to cause a genetic modification.

In some embodiments, the gene editing composition comprises one or more nucleic acids, wherein the one or more nucleic acids encode a gene editing protein and/or a guide RNA. In some embodiments, the gene editing composition comprises one or more nucleic acids, wherein the one or more nucleic acids encode a gene editing protein. In some embodiments, the gene editing composition comprises one or more nucleic acids, wherein the one or more nucleic acids encode a gene editing protein and a guide RNA.

In some embodiments the gene may be used to correct or ameliorate gene deficiencies, which may include deficiencies in which normal genes are expressed at less than normal levels or deficiencies in which the functional gene product is not expressed. Alternatively, the gene may provide a product to a cell which is not natively expressed in the cell type or in the host. A type of gene sequence encodes a therapeutic protein or polypeptide which is expressed in a host cell. The invention further includes using multiple genes. In certain situations, a different gene may be used to encode each subunit of a protein, or to encode different peptides or proteins. This is desirable when the size of the DNA encoding the protein subunit is large.

In some embodiments, the nucleic acid encodes a gene editing protein. A gene editing protein may be a Cas endonuclease, a Cpf1 endonuclease, a zinc finger nuclease, a transcription activator-like effector nuclease (TALEN), or a meganuclease.

A Cas endonuclease may be a Cas9 endonuclease, a dead Cas endonuclease (dCas, e.g., dCas9) In some embodiments, a Cas endonuclease is from Streptococcus pyogenes. A Cas endonuclease may be a wild-type Cas endonuclease or a modified or mutant versions of Cas endonuclease.

In some embodiments, the nucleic acid is a guide RNA. In some embodiments, a guide RNA is 20-200, 20-100, 50-200, 50-150, or about 100 nucleotides in length. In some embodiments, a guide RNA is a single-molecule guide RNA. In some embodiments, a guide RNA comprises a spacer sequence that binds to a target gene sequence. In some embodiments, a spacer sequence is 10, 11, 12, 13, 14, 15, 16, 17, 18, 19, 20, 21, 22, 23, 24, or 25 nucleotides in length.

In some embodiments, a method of gene editing further comprises delivery of a nucleic acid encoding a chimeric antigen receptor. A chimeric antigen receptor, in some embodiments, comprises an extracellular domain comprising an antigen binding domain (e.g., an antibody, such as an scFv), a transmembrane domain, and a cytoplasmic domain. In some embodiments, the extracellular domain of a chimeric antigen receptor includes an antigen binding domain and at least one of a signal peptide and/or a hinge domain. In some embodiments, the signal peptide enhances antigen specificity of the chimeric antigen receptor. In some embodiments, the hinge domain is located between an extracellular domain and the transmembrane domain of the chimeric antigen receptor. In some embodiments, the transmembrane domain is a hydrophobic alpha helix that spans cellular membrane to provide stability to the chimeric antigen receptor. In some embodiments, the cytoplasmic domain of the chimeric antigen receptor is a protein domain that, following antigen recognition, causes signal transduction within the cell.

Nucleic acids As used herein, the term “nucleic acids” generally refers to multiple linked nucleotides (i.e., molecules comprising a sugar (e.g., ribose or deoxyribose) linked to an exchangeable organic base, which is either a pyrimidine (e.g., cytosine (C), thymidine (T) or uracil (U)) or a purine (e.g., adenine (A) or guanine (G)). Nucleic acids include DNA such as D-form DNA and L-form DNA and RNA, as well as various modifications thereof. Modifications include base modifications, sugar modifications, and backbone modifications.

It is to be understood that the nucleic acids used in retroviruses and methods of the invention may be homogeneous or heterogeneous in nature. As an example, they may be completely DNA in nature or they may be comprised of DNA and non-DNA (e.g., LNA) monomers or sequences. Thus, any combination of nucleic acid elements may be used. The modification may render the nucleic acid more stable and/or less susceptible to degradation under certain conditions. For example, in some instances, the nucleic acids are nuclease-resistant. Methods for synthesizing nucleic acids, including automated nucleic acid synthesis, are also known in the art.

The nucleic acids may comprise modifications in their bases. Modified bases include modified cytosines (such as 5-substituted cytosines (e.g., 5-methyl-cytosine, 5-fluoro-cytosine, 5-chloro-cytosine, 5-bromo-cytosine, 5-iodo-cytosine, 5-hydroxy-cytosine, 5-hydroxymethyl-cytosine, 5-difluoromethyl-cytosine, and unsubstituted or substituted 5-alkynyl-cytosine), 6-substituted cytosines, N4-substituted cytosines (e.g., N4-ethyl-cytosine), 5-aza-cytosine, 2-mercapto-cytosine, isocytosine, pseudo-isocytosine, cytosine analogs with condensed ring systems (e.g., N,N′-propylene cytosine or phenoxazine), and uracil and its derivatives (e.g., 5-fluoro-uracil, 5-bromo-uracil, 5-bromovinyl-uracil, 4-thio-uracil, 5-hydroxy-uracil, 5-propynyl-uracil), modified guanines such as 7 deazaguanine, 7 deaza 7 substituted guanine (such as 7 deaza 7 (C2 C6)alkynylguanine), 7 deaza 8 substituted guanine, hypoxanthine, N2-substituted guanines (e.g. N2-methyl-guanine), 5-amino-3-methyl-3H,6H-thiazolo[4,5-d]pyrimidine-2,7-dione, 2,6 diaminopurine, 2 aminopurine, purine, indole, adenine, substituted adenines (e.g. N6-methyl-adenine, 8-oxo-adenine) 8 substituted guanine (e.g. 8 hydroxyguanine and 8 bromoguanine), and 6 thioguanine. The nucleic acids may comprise universal bases (e.g. 3-nitropyrrole, P-base, 4-methyl-indole, 5-nitro-indole, and K-base) and/or aromatic ring systems (e.g. fluorobenzene, difluorobenzene, benzimidazole or dichloro-benzimidazole, 1-methyl-1H-[1,2,4]triazole-3-carboxylic acid amide). A particular base pair that may be incorporated into the oligonucleotides of the invention is a dZ and dP non-standard nucleobase pair reported by Yang et al. NAR, 2006, 34(21):6095-6101. dZ, the pyrimidine analog, is 6-amino-5-nitro-3-(1′-13-D-2′-deoxyribofuranosyl)-2(1H)-pyridone, and its Watson-Crick complement dP, the purine analog, is 2-amino-8-(1′-13-D-1′-deoxyribofuranosyl)-imidazo[1,2-a]-1,3,5-triazin-4(8H)-one.

Amino acid substitutions

In some embodiments, the amino acid residue variations are conservative amino acid residue substitutions. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds.,

John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

Reporter

In some embodiments, the retroviruses described herein may comprise a reporter (e.g., a reporter protein). In some embodiments, the retroviruses described herein comprise a nucleic acid encoding a reporter (e.g., a reporter protein). As used herein, a reporter is generally a protein or gene that can be detected when expressed in a retrovirus and/or target cell. In some embodiments, the presence or absence of a reporter in a target cell or a subset of a target cells in a population of cells allows for the ability to sort cells (e.g., using flow cytometry and/or fluorescence-activated cell sorting).

In some embodiments, a reporter is a fluorescent protein. A fluorescent protein may be a green fluorescent protein (GFP), yellow fluorescent protein (YFP), red fluorescent protein (RFP). A fluorescent protein may be as described in U.S. Pat. No. 7,060,869, entitled “Fluorescent protein sensors for detection of analytes”.

In some embodiments, a reporter is an antibiotic resistance marker. In some embodiments, an antibiotic resistance marker is a protein or gene that confers a competitive advantage to a target cell that contains the marker. In some embodiments, the antibiotic resistance marker comprises a hygromycin resistance protein or gene, a kanamycin resistance protein or gene, ampicillin resistant protein or gene, streptromycin resistant protein or gene, or a neomycin resistance protein or gene.

Cells

A cell as described herein may be any bacterial, mammalian, or yeast cell. In some embodiments, a cell is a human, mouse, rat, or a non-human primate cell. In some embodiments, the cell is a stem cell. In some embodiments, the cell is a hematopoietic stem cell (HSC).

In some embodiments, a cell is a somatic cell or a reproductive cell. In some embodiments, a cell is an epithelial cell, a neural cell, a hormone-secreting cell, an immune cell, a secretory cell, a blood cell, an interstitial cell, or a germ cell. In some embodiments, a cell is an antigen-specific cell (e.g., a cell that binds to a specific antigen). In some embodiments, an antigen-specific cell is an immune cell. In some embodiments, an antigen-specific cell is a B cell or a T cell. In some embodiments, a cell is a target cell (e.g., that comprises a cognate protein or ligand capable of being targeted by a retrovirus described herein)

A population of cells as described herein may be any bacterial, mammalian, or yeast cell population. In some embodiments, a population of cells is a population of human, mouse, rat, or non-human primate cells. In some embodiments, a population of cells is a somatic cell population or a reproductive cell population. In some embodiments, a population of cells comprises epithelial cells, neural cells, hormone-secreting cells, immune cells, secretory cells, blood cells, interstitial cells, and/or germ cells. In some embodiments, a population of cells comprises antigen-specific cells (e.g., cells that binds to a specific antigen). In some embodiments, a population of antigen-specific cells comprises immune cells. In some embodiments, a population of antigen-specific cells comprises B cells and/or T cells. In some embodiments, a population of cells comprises a homogenous population of cells. In some embodiments, a population of cells comprises a heterogeneous population of cells.

In some embodiments, a population of cells is a population of cells isolated from a subject. A subject may be a human subject (e.g., a human subject suffering from a disease), a mouse subject, a rat subject, or a non-human primate subject. In some embodiments, a population of cells is isolated from the blood or a tumor of a subject.

In some embodiments, a population of cells has been previously frozen and thawed (e.g., 1, 2, 3, 4, 5, or more freeze/thaw cycles). In some embodiments, a population of cells are maintained in liquid culture media. In some embodiments, a population of cells have been passaged 1, 2, 3, 4, 5, or more times, using any known method. In some embodiments, a population of cells are maintained in liquid culture media prior to being combined with a retrovirus or plurality of retroviruses. In some embodiments, a population of cells are maintained in liquid culture media after to being combined with a retrovirus or plurality of retroviruses. In some embodiments, a population of cells are maintained in liquid culture media prior to while being combined with a retrovirus or plurality of retroviruses.

In some embodiments, a population of cells comprises any of the retroviruses described herein. In some embodiments, a subset of a population of cells contain any of the retroviruses described herein. In some embodiments, a subset of a population of cells contains the retrovirus inside each cell of the subset (e.g., inside the nucleus of each cell of the subset). In some embodiments, a population of cells or a subset thereof expresses a reporter (e.g., a fluorescent protein or an antibiotic resistance marker). In some embodiments, a population of cells or a subset thereof (e.g., containing a retrovirus) are isolated and/or sorted based on the presence or absence of a reporter. In some embodiments, a subset of a population of cells that contain retrovirus described herein are isolated and/or sorted based on the presence or absence of a reporter away from the cells of the population that do not contain the retrovirus. In some embodiments, at least 50%, 60%, 70%, 80%, 90%, or 95% of a population of cells prior to cell sorting contain a retrovirus. In some embodiments, at least 70%, 80%, 90%, 95%, or 100% of a population of cells contain a retrovirus following isolation and/or sorting based on the presence or absence of a reporter.

As used herein, the term “combining” (which, in some embodiments, is synonymous with the terms “providing” and “contacting”) generally refers to the act of bringing a retrovirus into close, physical contact with a population of cells, such that the extracellular targeting domain of the retrovirus is capable of binding to the cognate ligand present on a subset of cells of the population. In some embodiments, combining of a retrovirus and a population of cells occurs when a solution comprising the retrovirus and a solution comprising the population of cells are mixed. In some embodiments, combining of a retrovirus and a population of cells occurs when a lyophilized retrovirus and a solution comprising the population of cells are mixed. In some embodiments, combining of a retrovirus and a population of cells occurs when a lyophilized retrovirus and a lyophilized population of cells are mixed and reconstituted with a solution. In some embodiments, the cells of the population are maintained in cell culture media, in a monolayer of cells, and/or are attached to a tissue culture plate or petri dish.

Generally, a retrovirus and a population of cells are combined (e.g., physically combined or contacted) for a defined period of time. In some embodiments, a period of time is measured in seconds, minutes, hours or days. In some embodiments, period of time is 0-30 seconds, 15-45 seconds, 30-60 seconds, 45-90 seconds, 60-90 seconds, or 60-120 seconds. In some embodiments, a retrovirus and a population of cells are combined and in contact for 0-30 seconds, 15-45 seconds, 30-60 seconds, 45-90 seconds, 60-90 seconds, or 60-120 seconds. In some embodiments, period of time is 1-2 minutes, 1-5 minutes, 1-10 minutes, 2-10 minutes, 5-10 minutes, 5-20 minutes, 10-20 minutes, 25-30 minutes, 25-60 minutes, 30-45 minutes, 30-40 minutes, 40-60 minutes, 50-70 minutes, or 60-120 minutes. In some embodiments, a retrovirus and a population of cells are combined and in contact for 1-2 minutes, 1-5 minutes, 1-10 minutes, 2-10 minutes, 5-10 minutes, 5-20 minutes, 10-20 minutes, 25-30 minutes, 25-60 minutes, 30-45 minutes, 30-40 minutes, 40-60 minutes, 50-70 minutes, or 60-120 minutes. In some embodiments, a period of time is 1-2 hours, 1-5 hours, 1-3 hours, 2-5 hours, 3-6 hours, 3-12 hours, 6-12 hours, 12-18 hours, 12-24 hours, 15-30 hours, 18-24 hours, 24-48 hours, 24-36 hours, or 36-50 hours. In some embodiments, a retrovirus and a population of cells are combined and in contact for 1-2 hours, 1-5 hours, 1-3 hours, 2-5 hours, 3-6 hours, 3-12 hours, 6-12 hours, 12-18 hours, 12-24 hours, 15-30 hours, 18-24 hours, 24-48 hours, 24-36 hours, or 36-50 hours. In some embodiments, a period of time is 1, 2, 3, 4, 5, 6, 7, 8, 9, 10, or 5-15 days. In some embodiments, a retrovirus and a population of cells are combined and in contact for 1, 2, 3, 4, 5, 6, 7, 8, 9, 10, or 5-15 days.

In some embodiments, a population of cells are sorted based on the presence or absence of the reporter. In some embodiments, a subset of the population of cells containing the reporter (e.g., express the reporter) are sorted from the remaining subset of the population of cells that do not contain the reporter. In some embodiments, sorting of the population of cells is performed using flow cytometry (e.g., fluorescence-activated cell sorting), next-generation genome sequencing (e.g., single-cell next-generation sequencing), or antibiotic selection.

In some embodiments, the conditions of step (ii) that allow for the retrovirus to have cell-to-cell interactions with a subset of the population of cells comprise combining the retrovirus and the population of cells in the presence of defined solutions, compositions and at specific temperatures. In some embodiments, the retrovirus and the population of cells are combined in the presence of a cell culture media (e.g., RPMI or DMEM cell culture media). In some embodiments, the retrovirus and the population of cells are combined in the presence of a buffered saline solution. In some embodiments, a buffered saline solution is a phosphate-buffered saline or HEPES-buffered saline. In some embodiments, a buffered saline solution comprises bovine serum albumin and/or EDTA. In some embodiments, the retrovirus and the population of cells are combined in the presence of an enhancer of retroviral transduction (e.g., heparin sulfate, polybrene, protamine sulfate, or dextran). In some embodiments, the retrovirus and the population of cells are combined in (ii) at a temperature ranging from 4° C. to 42° C., 4° C. to 8° C., 4° C. to 10° C., 8° C. to 15° C., 10° C. to 20° C., 18° C. to 23° C., 20° C. to 30° C., 25° C. to 35° C., 30° C. to 40° C., or 37° C. to 42° C.

In some embodiments, the methods of screening described herein further comprise washing the population of cells between steps (ii) and (iii) with a wash solution. In some embodiments, a wash solution is any liquid solution that allows for maintenance of healthy cells (e.g., solution comprising neutral pH, low-to-moderate levels of ionic strength). In some embodiments, washing the population of cells removes excess and/or remaining retrovirus from the population of cells. In some embodiments, the population of cells are washed using a cell culture media (e.g., RPMI or DMEM cell culture media). In some embodiments, the population of cells are washed using a buffered saline solution. In some embodiments, a buffered saline solution is a phosphate-buffered saline or HEPES-buffered saline. In some embodiments, a buffered saline solution comprises bovine serum albumin and/or EDTA. In some embodiments, the population of cells are washed at a temperature ranging from 4° C. to 42° C., 4° C. to 8° C., 4° C. to 10° C., 8° C. to 15° C., 10° C. to 20° C., 18° C. to 23° C., 20° C. to 30° C., 25° C. to 35° C., 30° C. to 40° C., or 37° C. to 42° C.

In some embodiments, the population of cells are maintained in liquid culture prior to being combined with the retrovirus. In some embodiments, the population of cells are maintained in liquid culture after being combined with the retrovirus. In some embodiments, the population of cells are maintained in liquid culture during the combining step with the retrovirus. In some embodiments, the population of cells are attached to a cell culture plate or petri dish. In some embodiments, the population of cells are maintained in a monolayer, an embryoid body, or any cell aggregate.

In certain embodiments, a plurality of retroviruses comprises at least 10², 10³, 10⁴, 10⁵, 10⁶, 10⁷, 10⁸, 10⁹, 10¹⁰, 10¹¹, or 10¹² unique retroviruses. In some embodiments, there may be at least 10², 10³, 10⁴, 10⁵, 10⁶, 10⁷, 10⁸, 10⁹, 10¹⁰, 10¹¹, or 10¹² copies of each unique retrovirus present in a plurality of retroviruses.

Library of retroviruses

Described herein are libraries of retroviruses, wherein a library comprises a plurality of unique retroviruses, wherein each unique retrovirus comprises a viral envelope protein comprising at least one mutation that diminishes its native function, a non-viral membrane-bound protein comprising a membrane-bound domain and an extracellular targeting domain, and a nucleic acid encoding a reporter, and wherein each unique retrovirus comprises a different and unique extracellular targeting domain. Also described herein are libraries of cells comprising retroviruses, wherein a library comprises a plurality of unique cells, wherein each unique cell comprises a unique retrovirus.

In some embodiments, a library comprises at least 10², at least 10³, at least 10⁴, at least 10⁵, at least 10⁶, at least 10⁷, at least 10⁸, at least 10⁹, or at least 10¹⁰ unique retroviruses. In some embodiments, a library comprising unique retroviruses comprises extracellular targeting domains that are at least 5, at least 10, at least 15, at least 20, or at least 50 amino acids in length.

In some embodiments, each different and unique extracellular targeting domain is generated through site-directed mutagenesis.

Retroviral or cell libraries can vary in size from hundreds to hundreds of thousands, millions, or more unique retroviruses or unique cells. In some embodiments, the libraries of the disclosure comprise at least 500,000 unique retroviruses or unique cells. The libraries of the invention include retroviral libraries and cellular libraries. A library is a synthetic (i.e., isolated, synthetically produced, free from components that are naturally found together in a cell, purified before being put into the library) collection of members having a common element and at least one distinct element. The library comprises a thousand or more (e.g., at least: 1,000; 2,000; 3,000; 4,000; 5,000; 10,000; 50,000; 100,000; 500,000; 600,000; 700,000; 800,000; 900,000; 1,000,000; 2,000,000; 3,000,000; 4,000,000; or more) members. The upper limit of the library size is defined by the combinatorics of domains or modules providing distinctness or diversity among the members. For instance, an upper limit may be 4,000,000 members. Thus, in some embodiments, the library is highly diverse, and includes at least 500,000 distinct members. The highly diverse library may have a diversity of 10⁶ or greater. In some embodiments, a library of retroviruses is generated using site-directed mutagenesis of a nucleic acid described herein. In some embodiments, the site-directed mutagenesis involves the use of primers and a low-fidelity RNA polymerase to allow for randomized mutagenesis of a common nucleic acid as described herein.

Methods of detection

Described herein are methods of detecting an interaction between a retrovirus and a cell, comprising: (i) contacting a sample comprising the retrovirus and an cell with an antibody, wherein the retrovirus comprises a viral envelope protein comprising at least one mutation that diminishes its native function, a non-viral membrane-bound protein comprising an extracellular targeting domain, and wherein the antibody binds to the extracellular targeting domain of the retrovirus; (ii) optionally removing unbound antibody from the sample; and (iii) imaging the sample to detect whether the antibody-retrovirus complex is bound to the cell.

In some embodiments, the antibody further comprises at least one fluorescent label. In some embodiments, a fluorescent label is a xanthene derivative (e.g., fluorescein, rhodamine, Oregon green, eosin and Texas red), cyanine derivative (e.g., cyanine, indocarbocyanine, oxacarbocyanine, thiacarbocyanine and merocyanine), naphthalene derivative (e.g., dansyl and prodan derivatives), coumarin derivative, oxadiazole derivative (e.g., pyridyloxazole, nitrobenzoxadiazole and benzoxadiazole), pyrene derivative (e.g., cascade blue), oxazine derivative (e.g., Nile red, Nile blue, cresyl violet and oxazine 170), acridine derivative (e.g., proflavin, acridine orange and acridine yellow), arylmethine derivative (e.g., auramine, crystal violet and malachite green), or tetrapyrrole derivative (e.g., porphin, phthalocyanine and bilirubin). The fluorescent label may be non-covalently associated with the antibody or covalently linked to the antibody.

In some embodiments, the sample is imaged in step (iii) using confocal or fluorescence microscopy. In some embodiments, methods of detection can be accomplished using standard microscopy setups (e.g., confocal or fluorescence microscopes). In some embodiments, a sample is detected in an ultra-multiplexed format while imaging using standard confocal or epi-fluorescence microscope.

### Examples

### Example 1. Expression of SCFa and S4-3a PStalk and IgG4Hinge constructs

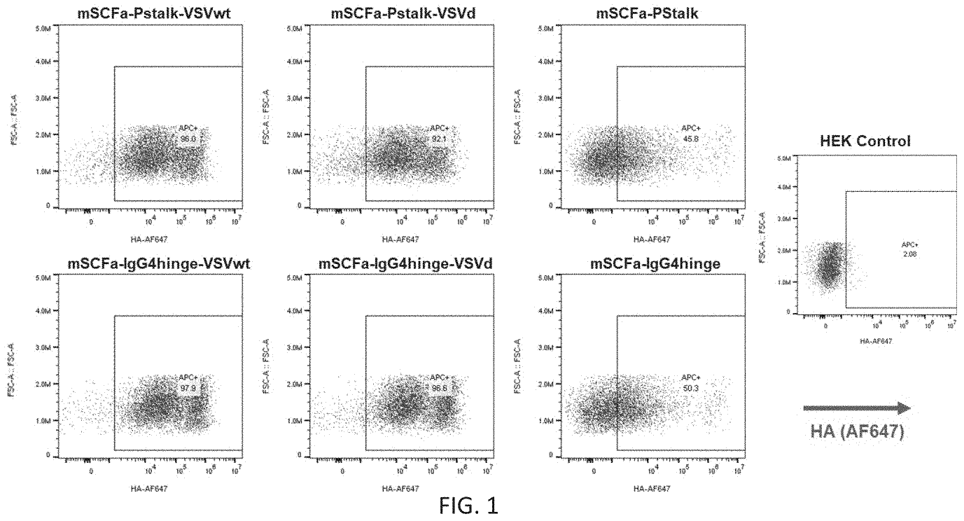

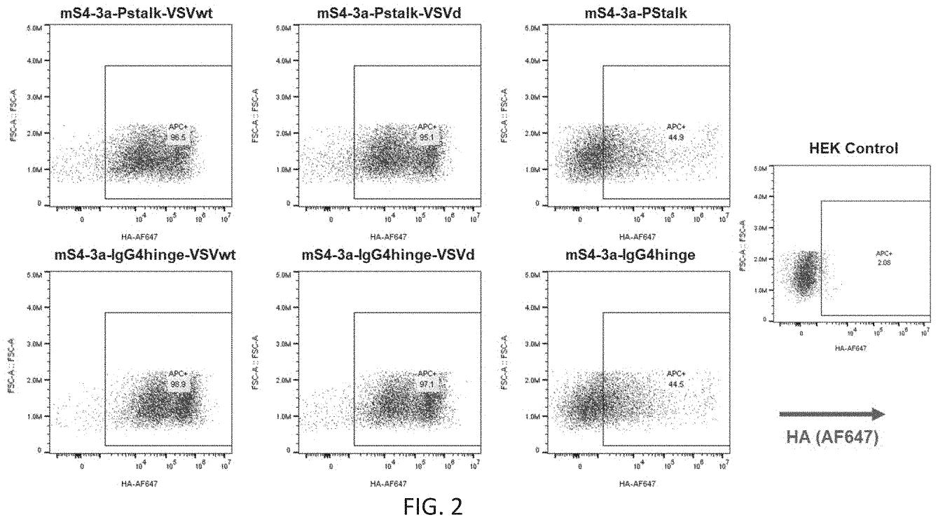

Targeted lentiviruses were generated by polyethylenimine (PEI) transfection of HEK293T cells using plasmids encoding a mutated VSV-G (VSVd) or wild-type VSV-G (VSVwt). Constructs containing wild-type murine stem cell factor (mSCFa),with endogenous affinity for the cKIT receptor, and S4-3a, an affinity matured version of SCF which has been shown to exhibit more efficient viral entry (Ho C C et al., Cell 2017) were generated using the procedure described in International Patent Publication WO 2020/236263. Monomeric and pre-dimeric versions of the constructs were created. In the monomeric versions, mSCF was tethered to the PDGFR stalk and transmembrane protein (mSCFa-Pstalk (SEQ ID NO: 32), mS4-3a-Pstalk (SEQ ID NO: 28)). In the pre-dimeric versions, mSCF was tethered to an IgG4 hinge linker protein (mSCFa-IgG4hinge (SEQ ID NO: 42), mS4-3a-IgG4hinge (SEQ ID NO: 36)). The constructs were exposed to VSVd or VSVwt along with a fluorescently labeled antibody (HA-tag: AF647) and tested for expression on the surface of a HEK viral packaging line. As shown in FIG. 1 (mSCFa) and FIG. 2 (mS4-3a), all the constructs were found to express on HEK cells.

### Example 2. Expression of unconcentrated virus in MC9 cKIT-expressing cells

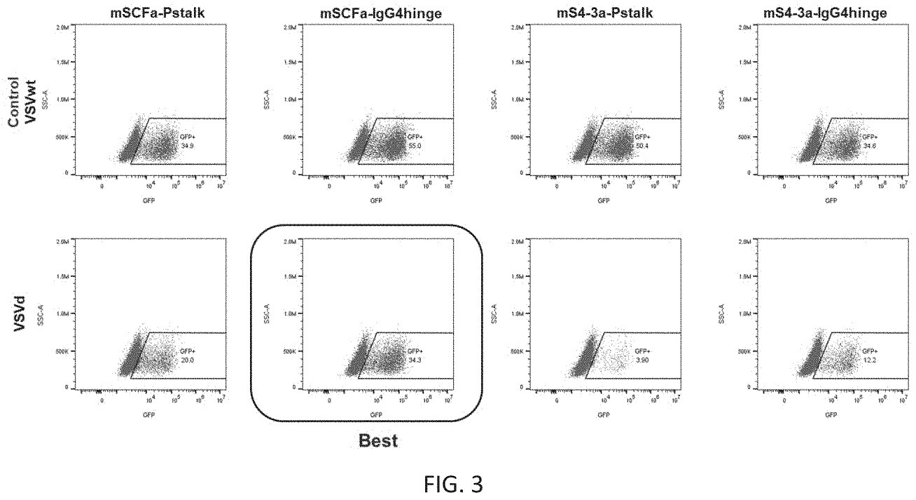

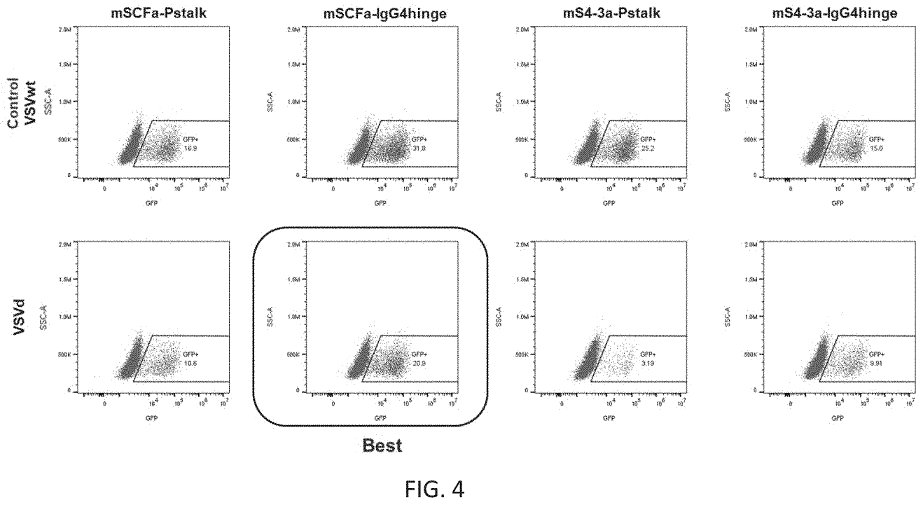

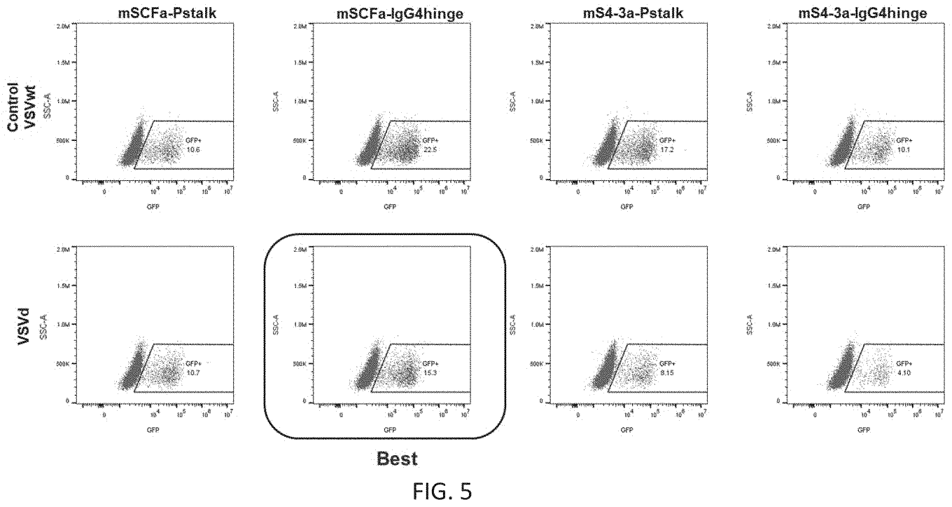

To test the cKIT affinity of the constructs described in Example 1, the constructs were tested for expression in MC9 cells. MC9 cells are not hematopoietic stem cells (HSCs). Instead, MC9 is a Mast Cell-based immortalized cell line that is cKIT+. MC9 cells were mixed via pipette mixing with unconcentrated VSVwt (control) and VSVd virus in the following ratios 1:1 (FIG. 3), 2:1 (FIG. 4) and 4:1 (FIG. 5). Results show that the SCF proteins enable selective viral entry into MC9 cells. The best performing constructs, mSCFa-IgG4hinge-VSVd and mS4-3a-IgG4hinge-VSVd, were selected for further experimentation.

### Example 3. Expression of concentrated virus with MC9 cKIT-expressing cells and VhCm non-cKIT-expressing cells

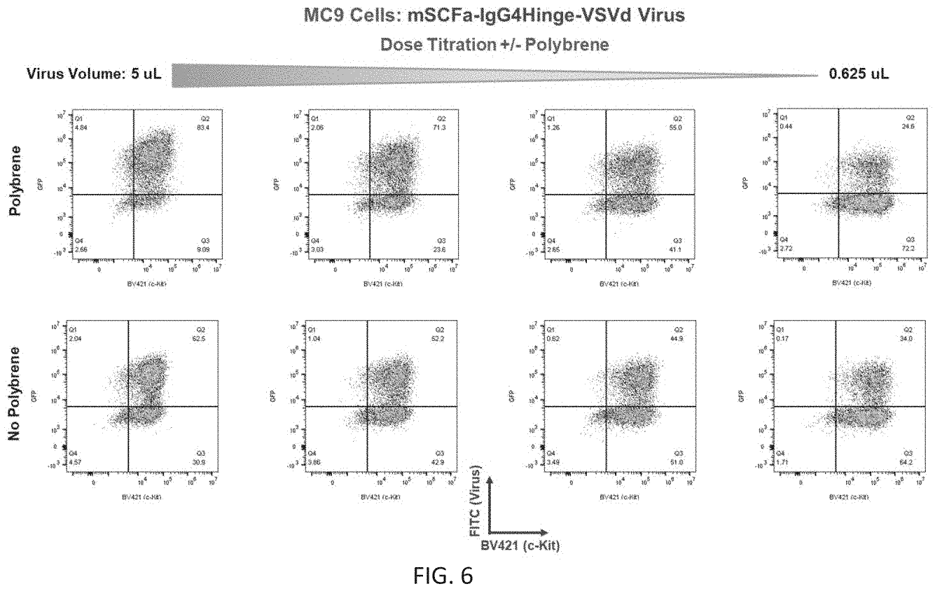

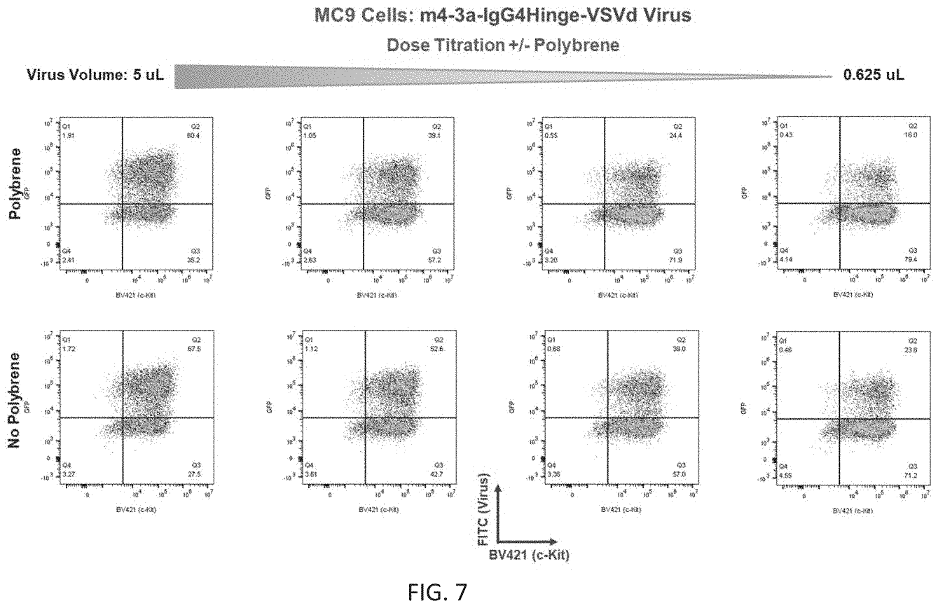

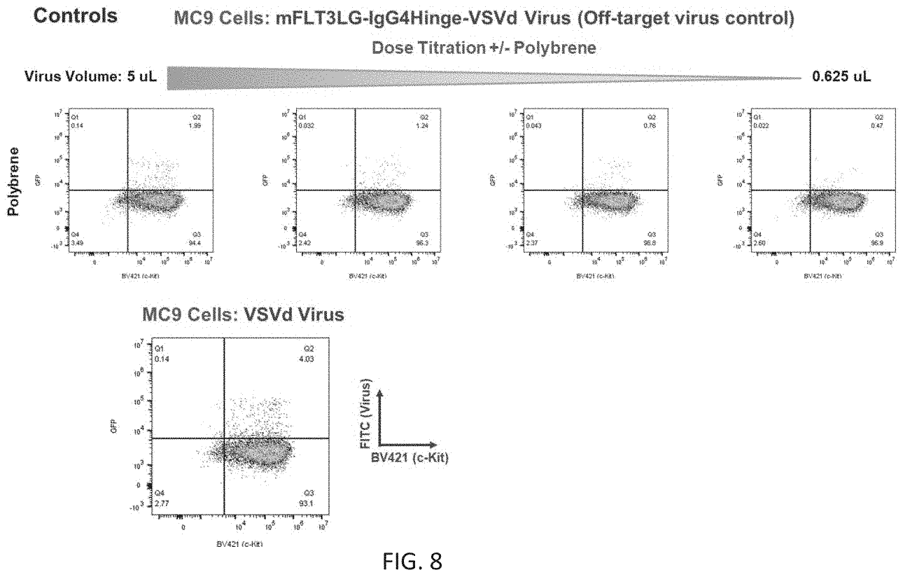

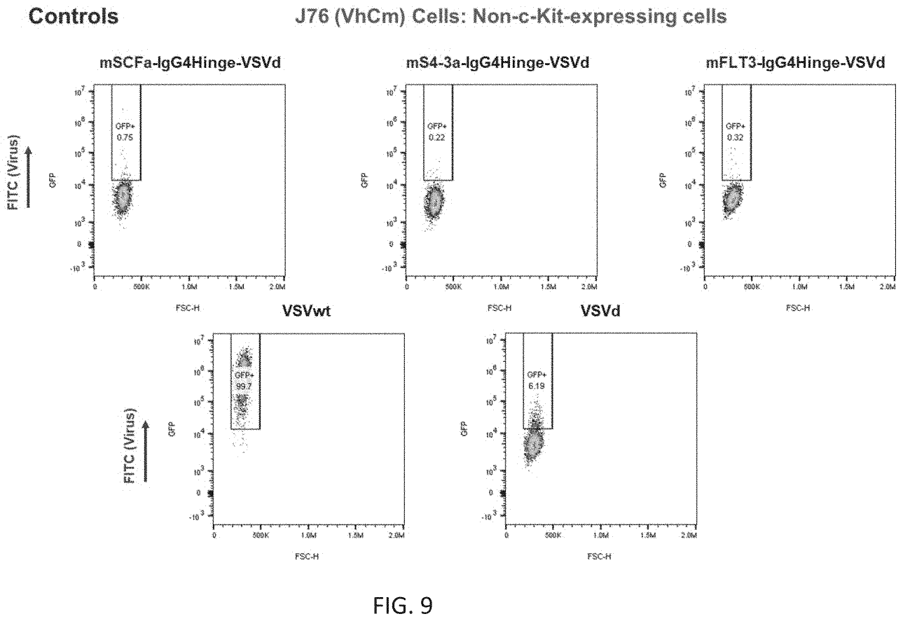

The lead constructs from Example 2 were further tested for viral entry into in MC9 (cKIT expressing) and VhCm (non-cKIT expressing) cell lines. Each construct was tested for MC9 viral entry at volumes of 5 uL, 2.5 uL 1.25 uL and 0.625 uL, in the presence or absence of polybrene, a retroviral transduction enhancer. Two markers were measured: FITC (to determine if virus was present) and BV421 (to determine the presence of cKIT). An off-target viral construct (mFLT3L-IgG4Hinge-VSVd) and the VSVd virus alone were used as controls. As shown in FIG. 8, the mFLT3LG virus did not bind cKIT and did not infect cKIT+ cells well. VSVd alone also did not infect cKIT+ cells well. All three constructs were also tested in J76 (VhCm cells) which are cKIT-. As shown in FIG. 9, even at the maximum dose of virus (5 uL) and in the presence of polybrene, none of the viruses infected cKIT-cells. Taken together, these results show that both mSCFa-IgG4hinge-VSVd (FIG. 6) and mS4-3a-IgG4hinge-VSVd (FIG. 7) enabled dose-dependent selective viral entry into cKIT+ cells.

### Example 4. Primary HSC transduction with engineered SCF and FLT3 specific virus

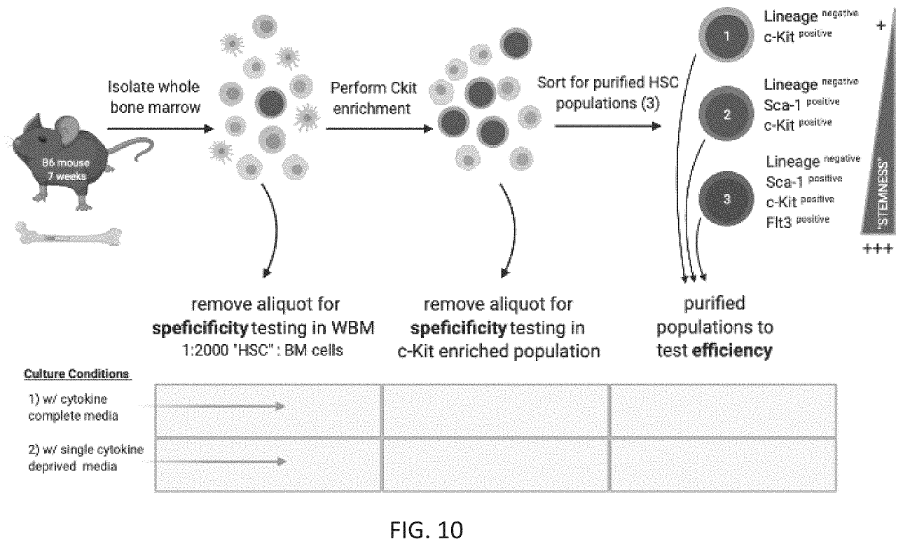

Engineered SCF and FLT3 virus constructs were tested to determine whether they were specific and efficient at delivering GFP protein to murine hematopoietic stem cells in the presence or absence of exogenous cytokines (SCF and FLT3) (FIG. 10).

Whole bone marrow cells (WBM) were isolated from B6 mice at 7 weeks. An aliquot of the isolated cells for was removed for further specificity testing in WBM. cKIT enrichment was performed and another aliquot was removed for further specificity testing in the cKIT enriched population. The cells were then sorted into three HSC populations according to the following criteria: 1-Lineage negative, cKIT positive; 2-Lineage negative, Sca-1 positive, cKIT positive (LSK); 3-Lineage negative, Sca-1 positive, cKIT positive, FLT3 positive. The cells were then cultured in media, with or without cytokines, for respective groups. The normal media for all HSC primary cells included FLT3L (50 ng/mL), TPO (50 ng/mL), and SCF (50 ng/mL). 24 hours after sorting, the cells (1M/mL) were incubated with concentrated virus at a ratio of 1:2. After 24 hours, the virus was removed, and cells were plated in cytokine complete media. 48 hours later, cells were stained, and flow panel was run to determine GFP expression within certain populations.

Efficiency and Specificity of SCF Virus Variants

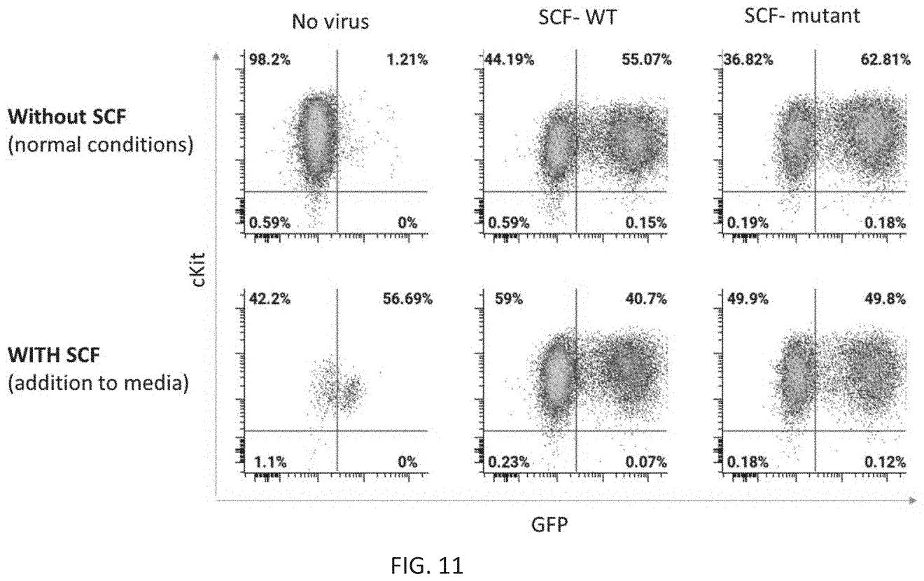

Positive controls were run using MC9 cells with SCF-WT and SCF mutant virus in media with and without SCF (FIG. 11). Results show that the concentrated virus had good transduction efficiency in MC9 control cells and that the addition of exogenous SCF into culture during transduction interfered with but did not completely inhibit viral delivery.

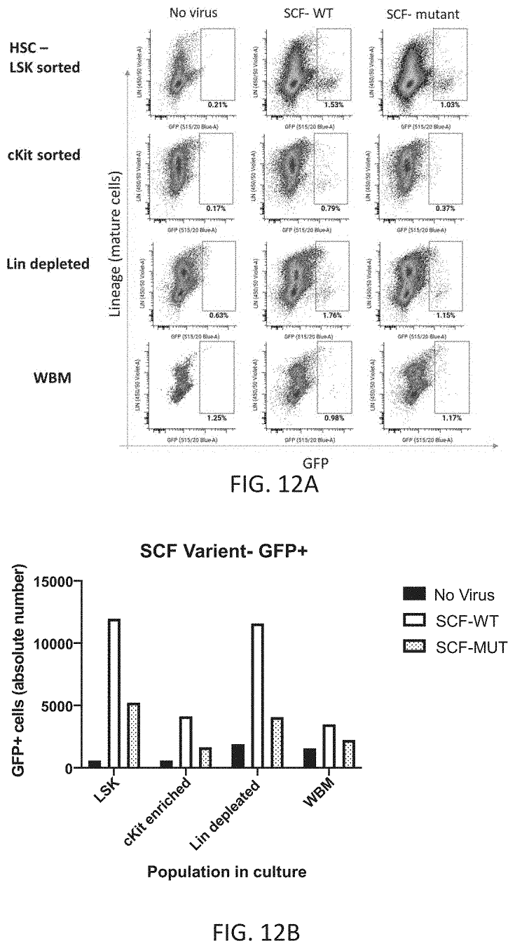

SCF-mutant was tested against SCF-WT in LSK (Lin-, Sca-1+, cKIT+), cKIT enriched, lineage depleted, and WBM (FIGS. 12A-12B). Results showed that GFP+ cells predominantly fell in the lineage—negative “immature” cell fraction. SCF-WT virus had slightly higher transduction efficiency than SCF-mutant. However, even in the purified population (LSK) efficiency was low.

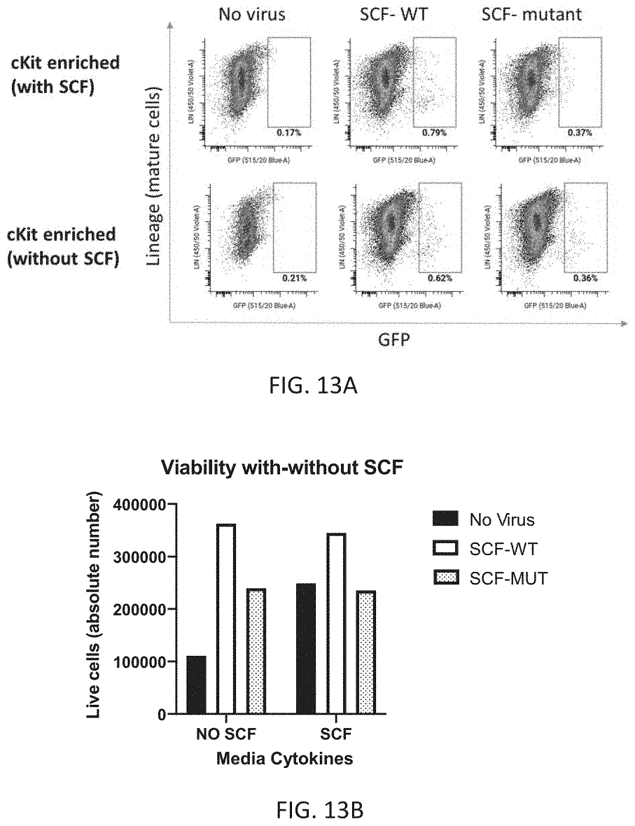

SCF specificity was then examined in cKIT enriched cells in media with and without SCF (FIGS. 13A-13B). Results show that viability and expansion was not greatly changed by short term culture depletion of SCF. Additionally, withholding SCF did not seem to significantly change % GFP-positive fraction

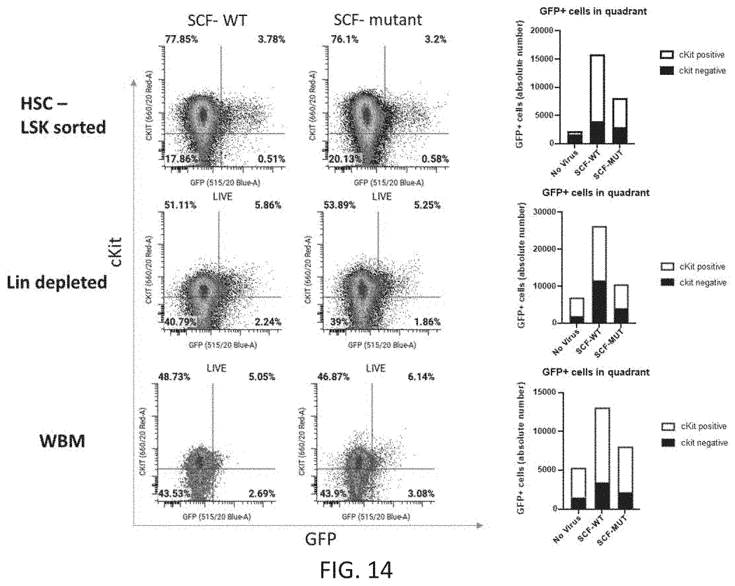

SCF virus specificity was determined in the LSK (Lin-, Sca-1+, cKIT+), lineage depleted, and WBM cell populations (FIG. 14). Results show GFP+ cells predominantly fell into the cKIT-positive quadrant. Additionally, it was shown that cKIT expression can be lost in culture, as all cells in LSK culture were at one-point cKIT+ and therefore the small fraction of GFP+cKIT-cells could have been due to a specific infection. Finally, relative specificity (#GFP+ cells that are cKIT+compared to cKIT-) scaled regardless of starting cells in culture. FLT3 Efficiency and Specificity

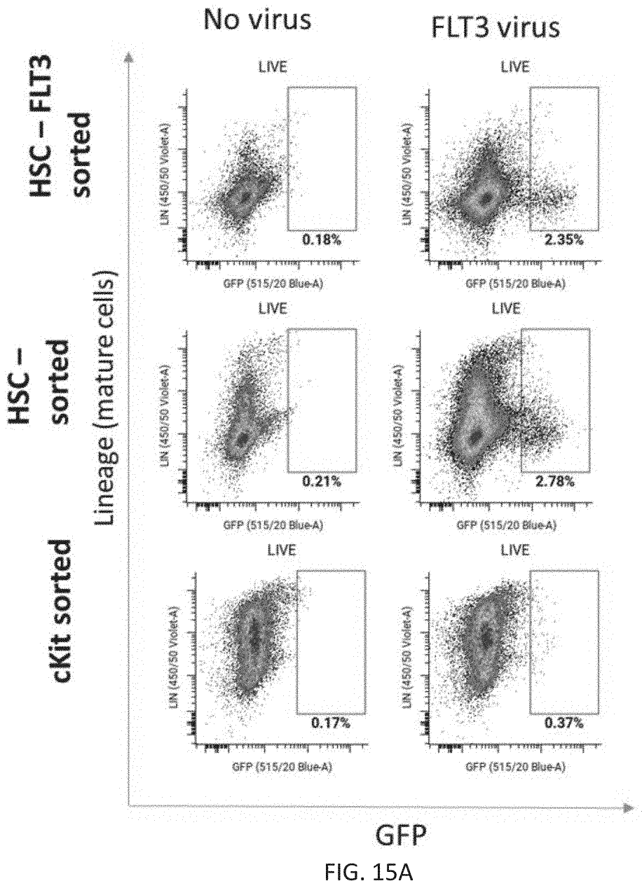

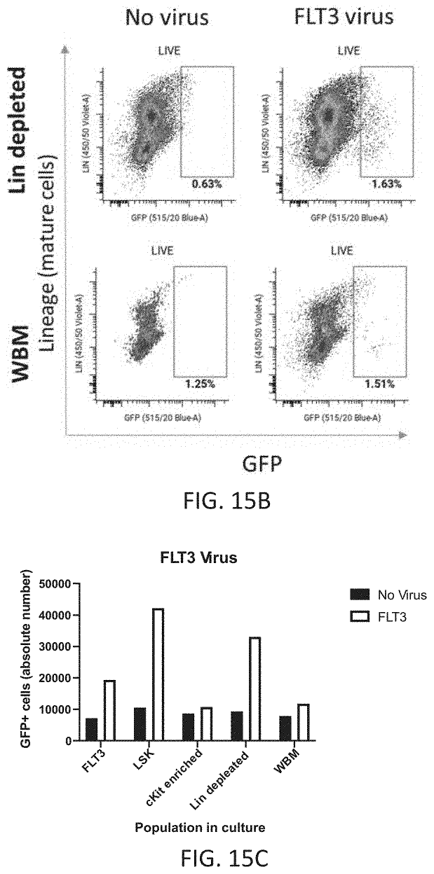

FLT3 virus efficiency was measured in HSC-FLT3 sorted, cKIT enriched, lineage depleted, and WBM cells (FIG. 15A-15C). Results show that FLT3 virus seemed to have slightly better efficiency than the SCF variants. Even in whole bone marrow, there was a small, but observable population that was effectively transduced.

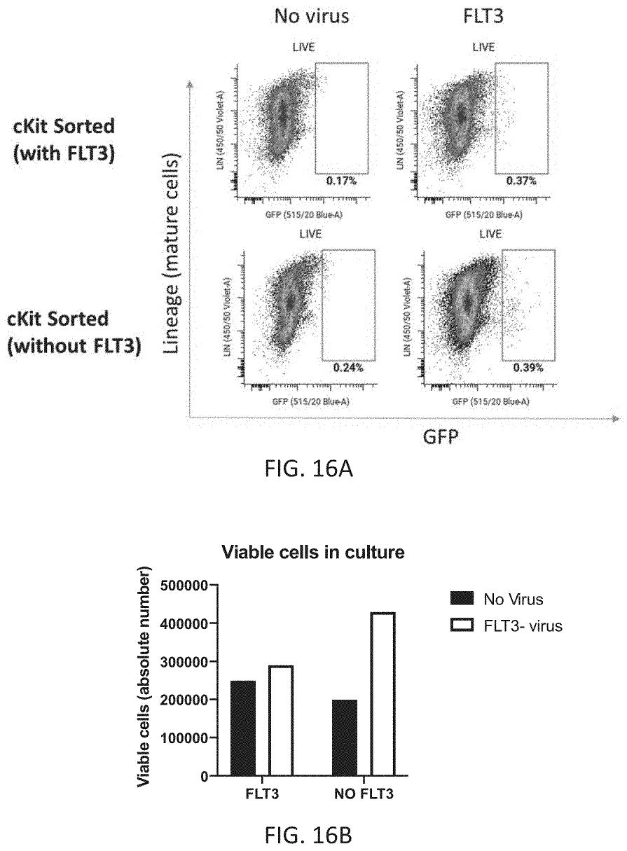

FLT3 specificity was then examined in cKIT enriched cells in media with and without FLT3 (FIGS. 16A-16B). Results show that viability and expansion was not greatly changed by short term culture depletion of FLT3. Additionally, results suggested that there may be slight benefit of withholding FLT3 in culture during transduction.

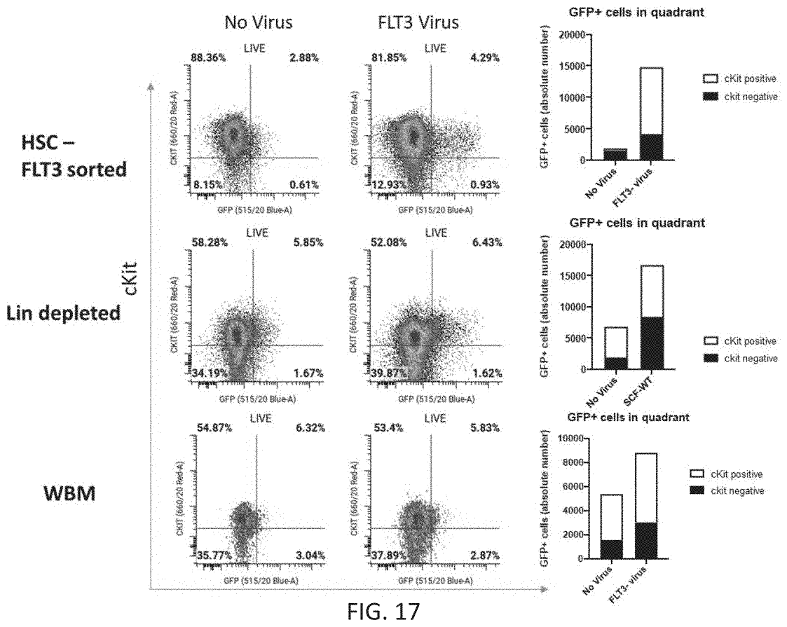

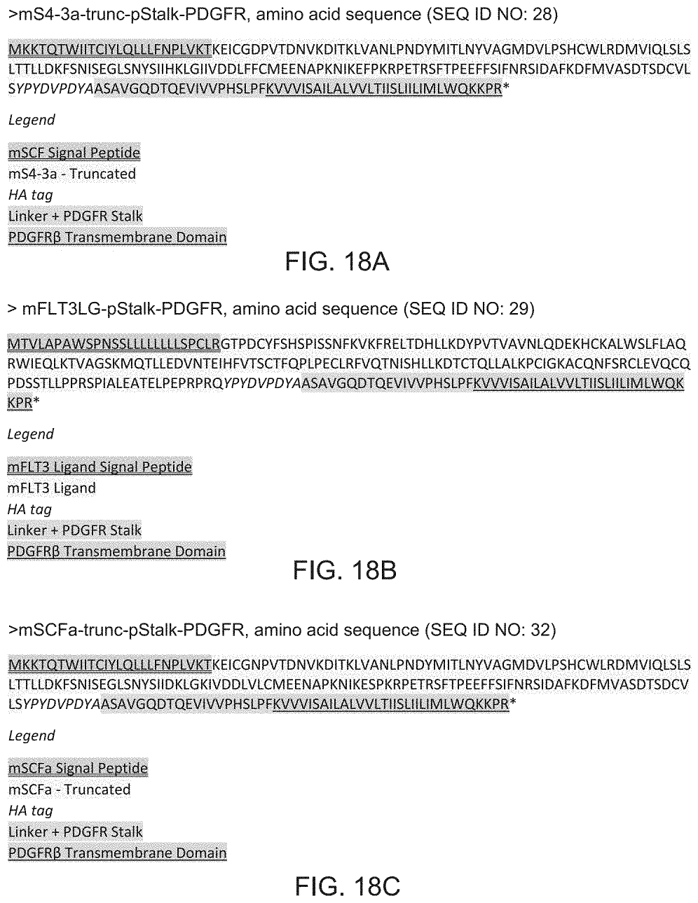

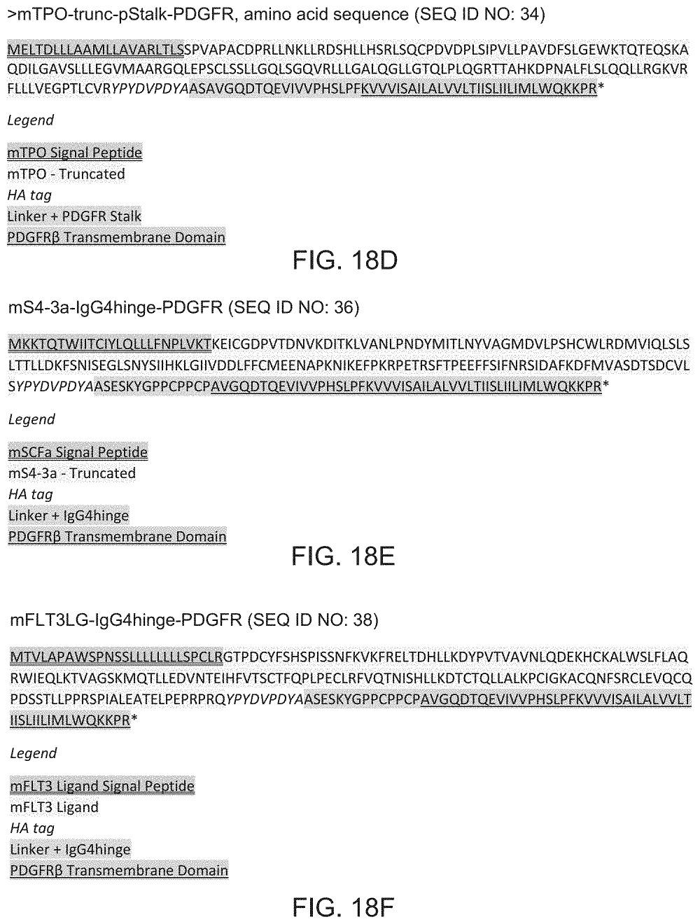

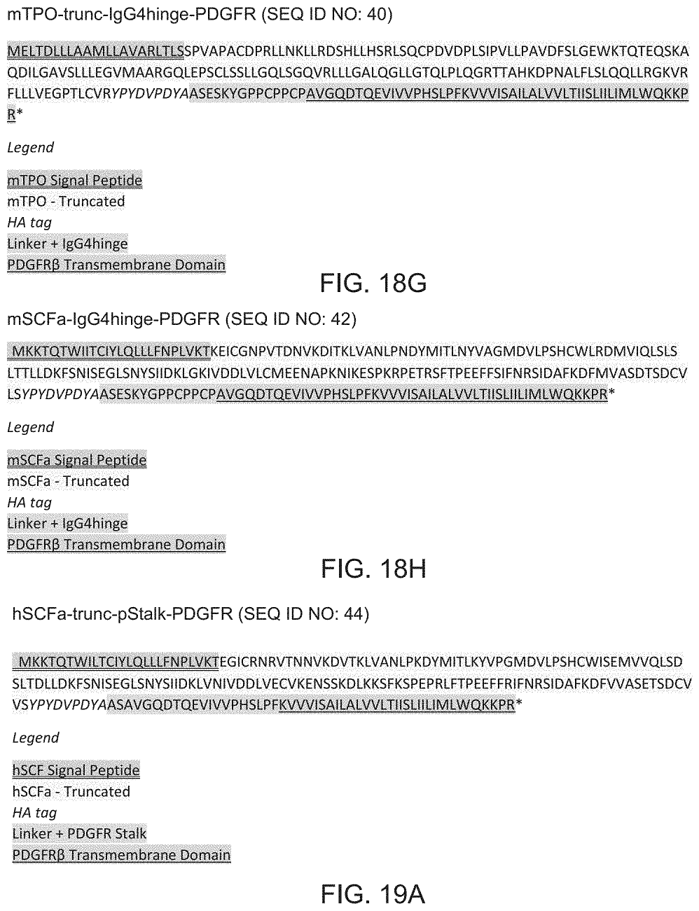

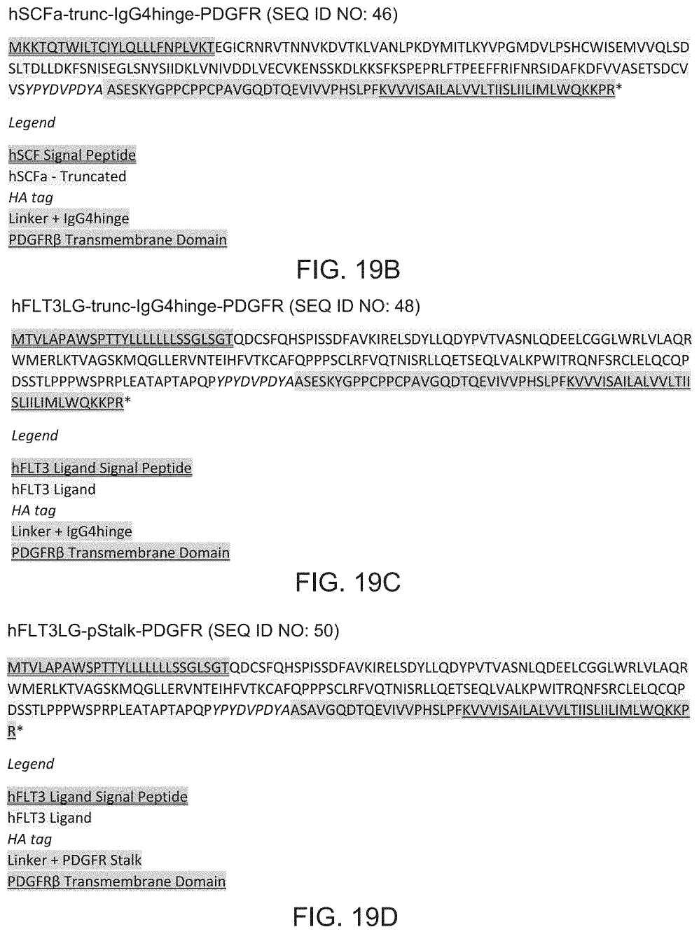

FLT3 virus specificity was also measured in the in the HSC-FLT3 sorted, lineage depleted, and WBM cell lines (FIG. 17). The FLT3 antibody stain did not look good, perhaps due to downregulation of FLT3 in culture. As a result, cKIT was used instead. Results showed that relative specificity (#GFP+ cells that are cKIT+compared to cKIT-) were good in purified populations but difficult to determine in WBM.

Taken together these results show that the engineered SCF and FLT3 lentiviruses demonstrate low efficiency transduction but fairly specific targeted integration. Although efficiency is low, there seems to be good specificity where cells that were successfully transduced (with both SCF and FLT3 viruses) are cKIT positive. Generally, the removal of a single cytokine from initial culture conditions did not seem to impede expansion and viability of cells. Overall, WBM did not perform well in culture conditions, suggesting that it may require a different set up for transduction-transplantation.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

SEQUENCES
>Kappa leader sequence, amino acid (SEQ ID NO: 1):
METDTLLLWVLLLWVPGSTG
>B2M signal peptide sequence, amino acid (SEQ ID NO: 2):
MSRSVALAVLALLSLSGLEA
>PDGFR short stalk, amino acid (SEQ ID NO: 3):
AVGQDTQEVIVVPHSLPFK
>PDGFR long stalk, amino acid (SEQ ID NO: 4):
ASAKPTTTPAPRPPTPAPTIASQPLSLRPEAARPAAGGAVHTRGLDFAK
>Short flexible linker, amino acid (SEQ ID NO: 5):
GAPGAS
>Long flexible linker, amino acid (SEQ ID NO: 6):
GAPGSGGGGSGGGGSAS
>Short flexible linker, amino acid (SEQ ID NO: 7):
GGGGS
>IgG4 hinge domain, amino acid (SEQ ID NO: 8):
ASESKYGPPCPPCPAVGQDTQEVIVVPHSLPFK
>Tetrameric coiled coil, amino acid (SEQ ID NO: 9):
ASGGGGSGELAAIKQELAAIKKELAAIKWELAAIKQGAG
>Dimeric coiled coil, amino acid (SEQ ID NO: 10):
ASESKYGPPCPPCP
>Wild-type VSV-G envelope protein (with leader sequence),
DNA sequence (SEQ ID NO: 11):
atgaagtgccttttgtacttagcctttttattcattggggtgaattgcaagttcaccatagtt
tttccacacaaccaaaaaggaaactggaaaaatgttccttctaattaccattattgcccgtca
agctcagatttaaattggcataatgacttaataggcacagccatacaagtcaaaatgcccaag
agtcacaaggctattcaagcagacggttggatgtgtcatgcttccaaatgggtcactacttgt
gatttccgctggtatggaccgaagtatataacacagtccatccgatccttcactccatctgta
gaacaatgcaaggaaagcattgaacaaacgaaacaaggaacttggctgaatccaggcttccct
cctcaaagttgtggatatgcaactgtgacggatgccgaagcagtgattgtccaggtgactcct
caccatgtgctggttgatgaatacacaggagaatgggttgattcacagttcatcaacggaaaa
tgcagcaattacatatgccccactgtccataactctacaacctggcattctgactataaggtc
aaagggctatgtgattctaacctcatttccatggacatcaccttcttctcagaggacggagag
ctatcatccctgggaaaggagggcacagggttcagaagtaactactttgcttatgaaactgga
ggcaaggcctgcaaaatgcaatactgcaagcattggggagtcagactcccatcaggtgtctgg
ttcgagatggctgataaggatctctttgctgcagccagattccctgaatgcccagaagggtca
agtatctctgctccatctcagacctcagtggatgtaagtctaattcaggacgttgagaggatc
ttggattattccctctgccaagaaacctggagcaaaatcagagcgggtcttccaatctctcca
gtggatctcagctatcttgctcctaaaaacccaggaaccggtcctgctttcaccataatcaat
ggtaccctaaaatactttgagaccagatacatcagagtcgatattgctgctccaatcctctca
agaatggtcggaatgatcagtggaactaccacagaaagggaactgtgggatgactgggcacca
tatgaagacgtggaaattggacccaatggagttctgaggaccagttcaggatataagtttcct
ttatacatgattggacatggtatgttggactccgatcttcatcttagctcaaaggctcaggtg
ttcgaacatcctcacattcaagacgctgcttcgcaacttcctgatgatgagagtttatttttt
ggtgatactgggctatccaaaaatccaatcgagcttgtagaaggttggttcagtagttggaaa
agctctattgcctcttttttctttatcatagggttaatcattggactattcttggttctccga
gttggtatccatctttgcattaaattaaagcacaccaagaaaagacagatttatacagacata
gagatgaaccgacttggaaagtaa
>Wild-type VSV-G envelope protein (with leader sequence),
amino acid sequence (SEQ ID NO: 12):
MKCLLYLAFLFIGVNCKFTIVFPHNQKGNWKNVPSNYHYCPSSSDLNWHNDLIGTAIQVKMPK
SHKAIQADGWMCHASKWVTTCDFRWYGPKYITQSIRSFTPSVEQCKESIEQTKQGTWLNPGFP
PQSCGYATVTDAEAVIVQVTPHHVLVDEYTGEWVDSQFINGKCSNYICPTVHNSTTWHSDYKV
KGLCDSNLISMDITFFSEDGELSSLGKEGTGFRSNYFAYETGGKACKMQYCKHWGVRLPSGVW
FEMADKDLFAAARFPECPEGSSISAPSQTSVDVSLIQDVERILDYSLCQETWSKIRAGLPISP
VDLSYLAPKNPGTGPAFTIINGTLKYFETRYIRVDIAAPILSRMVGMISGTTTERELWDDWAP
YEDVEIGPNGVLRTSSGYKFPLYMIGHGMLDSDLHLSSKAQVFEHPHIQDAASQLPDDESLFF
GDTGLSKNPIELVEGWFSSWKSSIASFFFIIGLIIGLFLVLRVGIHLCIKLKHTKKRQIYTDI
EMNRLGK
>Wild-type VSV-G envelope protein (without leader
sequence), amino acid sequence (SEQ ID NO: 13):
KFTIVFPHNQKGNWKNVPSNYHYCPSSSDLNWHNDLIGTAIQVKMPKSHKAIQADGWMCHASK
WVTTCDFRWYGPKYITQSIRSFTPSVEQCKESIEQTKQGTWLNPGFPPQSCGYATVTDAEAVI
VQVTPHHVLVDEYTGEWVDSQFINGKCSNYICPTVHNSTTWHSDYKVKGLCDSNLISMDITFF
SEDGELSSLGKEGTGFRSNYFAYETGGKACKMQYCKHWGVRLPSGVWFEMADKDLFAAARFPE
CPEGSSISAPSQTSVDVSLIQDVERILDYSLCQETWSKIRAGLPISPVDLSYLAPKNPGTGPA
FTIINGTLKYFETRYIRVDIAAPILSRMVGMISGTTTERELWDDWAPYEDVEIGPNGVLRTSS
GYKFPLYMIGHGMLDSDLHLSSKAQVFEHPHIQDAASQLPDDESLFFGDTGLSKNPIELVEGW
FSSWKSSIASFFFIIGLIIGLFLVLRVGIHLCIKLKHTKKRQIYTDIEMNRLGK
>VSV-G envelope protein (with leader sequence),
DNA sequence (SEQ ID NO: 14):
atgaagtgccttttgtacttagcctttttattcattggggtgaattgcaagttcaccatagtt
tttccacacaaccaaaaaggaaactggaaaaatgttccttctaattaccattattgcccgtca
agctcagatttaaattggcataatgacttaataggcacagccttacaagtcaaaatgccccag
agtcacaaggctattcaagcagacggttggatgtgtcatgcttccaaatgggtcactacttgt
gatttccgctggtatggaccgaagtatataacacagtccatccgatccttcactccatctgta
gaacaatgcaaggaaagcattgaacaaacgaaacaaggaacttggctgaatccaggcttccct
cctcaaagttgtggatatgcaactgtgacggatgccgaagcagtgattgtccaggtgactcct
caccatgtgctggttgatgaatacacaggagaatgggttgattcacagttcatcaacggaaaa
tgcagcaattacatatgccccactgtccataactctacaacctggcattctgactataaggtc
aaagggctatgtgattctaacctcatttccatggacatcaccttcttctcagaggacggagag
ctatcatccctgggaaaggagggcacagggttcagaagtaactactttgcttatgaaactgga
ggcaaggcctgcaaaatgcaatactgcaagcattggggagtcagactcccatcaggtgtctgg
ttcgagatggctgataaggatctctttgctgcagccagattccctgaatgcccagaagggtca
agtatctctgctccatctcagacctcagtggatgtaagtctaattcaggacgttgagaggatc
ttggattattccctctgccaagaaacctggagcaaaatcagagcgggtcttccaatctctcca
gtggatctcagctatcttgctcctaaaaacccaggaaccggtcctgctttcaccataatcaat
ggtaccctaaaatactttgagaccagatacatcagagtcgatattgctgctccaatcctctca
agaatggtcggaatgatcagtggaactaccacagaagccgaactgtgggatgactgggcacca
tatgaagacgtggaaattggacccaatggagttctgaggaccagttcaggatataagtttcct
ttatacatgattggacatggtatgttggactccgatcttcatcttagctcaaaggctcaggtg
ttcgaacatcctcacattcaagacgctgcttcgcaacttcctgatgatgagagtttatttttt
ggtgatactgggctatccaaaaatccaatcgagcttgtagaaggttggttcagtagttggaaa
agctctattgcctcttttttctttatcatagggttaatcattggactattcttggttctccga
gttggtatccatctttgcattaaattaaagcacaccaagaaaagacagatttatacagacata
gagatgaaccgacttggaaagtaa
>I41L/K47Q/R354A VSV-G envelope protein (with leader
sequence), amino acid sequence (SEQ ID NO: 15):
MKCLLYLAFLFIGVNCKFTIVFPHNQKGNWKNVPSNYHYCPSSSDLNWHNDLIGTALQVKMPQ
SHKAIQADGWMCHASKWVTTCDFRWYGPKYITQSIRSFTPSVEQCKESIEQTKQGTWLNPGFP
PQSCGYATVTDAEAVIVQVTPHHVLVDEYTGEWVDSQFINGKCSNYICPTVHNSTTWHSDYKV
KGLCDSNLISMDITFFSEDGELSSLGKEGTGFRSNYFAYETGGKACKMQYCKHWGVRLPSGVW
FEMADKDLFAAARFPECPEGSSISAPSQTSVDVSLIQDVERILDYSLCQETWSKIRAGLPISP
VDLSYLAPKNPGTGPAFTIINGTLKYFETRYIRVDIAAPILSRMVGMISGTTTEAELWDDWAP
YEDVEIGPNGVLRTSSGYKFPLYMIGHGMLDSDLHLSSKAQVFEHPHIQDAASQLPDDESLFF
GDTGLSKNPIELVEGWFSSWKSSIASFFFIIGLIIGLFLVLRVGIHLCIKLKHTKKRQIYTDI
EMNRLGK
>I41L/K47Q/R354A VSV-G envelope protein (without leader
sequence), amino acid sequence (SEQ ID NO: 16):
KFTIVFPHNQKGNWKNVPSNYHYCPSSSDLNWHNDLIGTALQVKMPQSHKAIQADGWMCHASK
WVTTCDFRWYGPKYITQSIRSFTPSVEQCKESIEQTKQGTWLNPGFPPQSCGYATVTDAEAVI
VQVTPHHVLVDEYTGEWVDSQFINGKCSNYICPTVHNSTTWHSDYKVKGLCDSNLISMDITFF
SEDGELSSLGKEGTGFRSNYFAYETGGKACKMQYCKHWGVRLPSGVWFEMADKDLFAAARFPE
CPEGSSISAPSQTSVDVSLIQDVERILDYSLCQETWSKIRAGLPISPVDLSYLAPKNPGTGPA
FTIINGTLKYFETRYIRVDIAAPILSRMVGMISGTTTEAELWDDWAPYEDVEIGPNGVLRTSS
GYKFPLYMIGHGMLDSDLHLSSKAQVFEHPHIQDAASQLPDDESLFFGDTGLSKNPIELVEGW
FSSWKSSIASFFFIIGLIIGLFLVLRVGIHLCIKLKHTKKRQIYTDIEMNRLGK
>K47Q/R354A VSV-G envelope protein (without leader
sequence), amino acid sequence (SEQ ID NO: 17):
KFTIVFPHNQKGNWKNVPSNYHYCPSSSDLNWHNDLIGTAIQVKMPQSHKAIQADGWMCHASK
WVTTCDFRWYGPKYITQSIRSFTPSVEQCKESIEQTKQGTWLNPGFPPQSCGYATVTDAEAVI
VQVTPHHVLVDEYTGEWVDSQFINGKCSNYICPTVHNSTTWHSDYKVKGLCDSNLISMDITFF
SEDGELSSLGKEGTGFRSNYFAYETGGKACKMQYCKHWGVRLPSGVWFEMADKDLFAAARFPE
CPEGSSISAPSQTSVDVSLIQDVERILDYSLCQETWSKIRAGLPISPVDLSYLAPKNPGTGPA
FTIINGTLKYFETRYIRVDIAAPILSRMVGMISGTTTEAELWDDWAPYEDVEIGPNGVLRTSS
GYKFPLYMIGHGMLDSDLHLSSKAQVFEHPHIQDAASQLPDDESLFFGDTGLSKNPIELVEGW
FSSWKSSIASFFFIIGLIIGLFLVLRVGIHLCIKLKHTKKRQIYTDIEMNRLGK
>Exemplary wild-type measles envelope protein (with
leader sequence), DNA sequence (SEQ ID NO: 18):
ATGGGCAGCCGGATCGTGATCAACCGGGAGCACCTGATGATCGACCGGCCCTACGTGCTGCTG
GCCGTGCTGTTCGTGATGTTCCTGAGCCTGATCGGCTTGCTAGCCATTGCTGGAATCCGGCTG
CACAGAGCCGCCATCTACACCGCCGAGATCCACAAGAGCCTGAGCACCAACCTGGACGTGACC
AACAGCATCGAGCATCAGGTCAAGGACGTGCTGACCCCCCTGTTTAAGATCATCGGCGACGAA
GTGGGCCTGCGGACCCCCCAGAGATTCACCGACCTGGTCAAGTTCATCAGCGACAAGATCAAG
TTCCTGAACCCCGACCGGGAGTACGACTTCCGGGACCTGACCTGGTGCATCAACCCCCCCGAG
CGGATCAAGCTGGACTACGACCAGTACTGCGCCGATGTGGCCGCCGAGGAACTGATGAATGCA
TTGGTGAACTCAACTCTACTGGAGACCAGAACAACCAATCAGTTCCTAGCTGTCTCAAAGGGA
AACTGCTCAGGGCCCACTACAATCAGAGGTCAATTCTCAAACATGTCGCTGTCCCTGTTAGAC
TTGTATTTAGGTCGAGGTTACAATGTGTCATCTATAGTCACTATGACATCCCAGGGAATGTAT
GGGGGAACTTACCTAGTGGAAAAGCCTAATCTGAGCAGCAAAAGGTCAGAGTTGTCACAACTG
AGCATGTACCGAGTGTTTGAAGTAGGTGTTATCAGAAATCCGGGTTTGGGGGCTCCGGTGTTC
CATATGACAAACTATCTTGAGCAACCAGTCAGTAATGATCTCAGCAACTGTATGGTGGCTTTG
GGGGAGCTCAAACTCGCAGCCCTTTGTCACGGGGAAGATTCTATCACAATTCCCTATCAGGGA
TCAGGGAAAGGTGTCAGCTTCCAGCTCGTCAAGCTAGGTGTCTGGAAATCCCCAACCGACATG
CAATCCTGGGTCCCCTTATCAACGGATGATCCAGTGATAGACAGGCTTTACCTCTCATCTCAC
AGAGGTGTTATCGCTGACAACCAAGCAAAATGGGCTGTCCCGACAACACGAACAGATGACAAG
TTGCGAATGGAGACATGCTTCCAACAGGCGTGTAAGGGTAAAATCCAAGCACTCTGCGAGAAT
CCCGAGTGGGCACCATTGAAGGATAACAGGATTCCTTCATACGGGGTCTTGTCTGTTGATCTG
AGTCTGACAGTTGAGCTTAAAATCAAAATTGCTTCGGGATTCGGGCCATTGATCACACACGGT
TCAGGGATGGACCTATACAAATCCAACCACAACAATGTGTATTGGCTGACTATCCCGCCAATG
AAGAACCTAGCCTTAGGTGTAATCAACACATTGGAGTGGATACCGAGATTCAAGGTTAGTCCC
tatCTCTTCAcaGTCCCAATTAAGGAAGCAGGCGGAGACTGCCATGCCCCAACATACCTACCT
GCGGAGGTGGATGGTGATGTCAAACTCAGTTCCAATCTGGTGATTCTACCTGGTCAAGATCTC
CAATATGTTTTGGCAACCTACGATACTTCCcgGGTTGAACATGCTGTGGTTTATTACGTTTAC
AGCCCAAGCCGCTCATTTTCTTACTTTTATCCTTTTAGGTTGCCTATAAAGGGGGTCCCCATC
GAATTACAAGTGGAATGCTTCACATGGGACCAAAAACTCTGGTGCCGTCACTTCTGTGTGCTT
GCGGACTCAGAATCTGGTGGACATATCACTCACTCTGGGATGGTGGGCATGGGAGTCAGCTGC
ACAGTCACCCGGGAAGATGGAACCAATGACTACAAAGACGATGACGACAAGTGA
>Exemplary wild-type measles envelope protein (with
leader sequence), amino acid sequence (SEQ ID NO: 19):
MGSRIVINREHLMIDRPYVLLAVLFVMFLSLIGLLAIAGIRLHRAAIYTAEIHKSLSTNLDVT
NSIEHQVKDVLTPLFKIIGDEVGLRTPQRFTDLVKFISDKIKFLNPDREYDFRDLTWCINPPE
RIKLDYDQYCADVAAEELMNALVNSTLLETRTTNQFLAVSKGNCSGPTTIRGQFSNMSLSLLD
LYLGRGYNVSSIVTMTSQGMYGGTYLVEKPNLSSKRSELSQLSMYRVFEVGVIRNPGLGAPVF
HMTNYLEQPVSNDLSNCMVALGELKLAALCHGEDSITIPYQGSGKGVSFQLVKLGVWKSPTDM
QSWVPLSTDDPVIDRLYLSSHRGVIADNQAKWAVPTTRTDDKLRMETCFQQACKGKIQALCEN
PEWAPLKDNRIPSYGVLSVDLSLTVELKIKIASGFGPLITHGSGMDLYKSNHNNVYWLTIPPM
KNLALGVINTLEWIPRFKVSPYLFTVPIKEAGGDCHAPTYLPAEVDGDVKLSSNLVILPGQDL
QYVLATYDTSRVEHAVVYYVYSPSRSFSYFYPFRLPIKGVPIELQVECFTWDQKLWCRHFCVL
ADSESGGHITHSGMVGMGVSCTVTREDGTNDYKDDDDK
>Exemplary mutant measles envelope protein (with leader
sequence), DNA sequence (SEQ ID NO: 20):
ATGGGCAGCCGGATCGTGATCAACCGGGAGCACCTGATGATCGACCGGCCCTACGTGCTGCTG
GCCGTGCTGTTCGTGATGTTCCTGAGCCTGATCGGCTTGCTAGCCATTGCTGGAATCCGGCTG
CACAGAGCCGCCATCTACACCGCCGAGATCCACAAGAGCCTGAGCACCAACCTGGACGTGACC
AACAGCATCGAGCATCAGGTCAAGGACGTGCTGACCCCCCTGTTTAAGATCATCGGCGACGAA
GTGGGCCTGCGGACCCCCCAGAGATTCACCGACCTGGTCAAGTTCATCAGCGACAAGATCAAG
TTCCTGAACCCCGACCGGGAGTACGACTTCCGGGACCTGACCTGGTGCATCAACCCCCCCGAG
CGGATCAAGCTGGACTACGACCAGTACTGCGCCGATGTGGCCGCCGAGGAACTGATGAATGCA
TTGGTGAACTCAACTCTACTGGAGACCAGAACAACCAATCAGTTCCTAGCTGTCTCAAAGGGA
AACTGCTCAGGGCCCACTACAATCAGAGGTCAATTCTCAAACATGTCGCTGTCCCTGTTAGAC
TTGTATTTAGGTCGAGGTTACAATGTGTCATCTATAGTCACTATGACATCCCAGGGAATGTAT
GGGGGAACTTACCTAGTGGAAAAGCCTAATCTGAGCAGCAAAAGGTCAGAGTTGTCACAACTG
AGCATGTACCGAGTGTTTGAAGTAGGTGTTATCAGAAATCCGGGTTTGGGGGCTCCGGTGTTC
CATATGACAAACTATCTTGAGCAACCAGTCAGTAATGATCTCAGCAACTGTATGGTGGCTTTG
GGGGAGCTCAAACTCGCAGCCCTTTGTCACGGGGAAGATTCTATCACAATTCCCTATCAGGGA
TCAGGGAAAGGTGTCAGCTTCCAGCTCGTCAAGCTAGGTGTCTGGAAATCCCCAACCGACATG
CAATCCTGGGTCCCCTTATCAACGGATGATCCAGTGATAGACAGGCTTTACCTCTCATCTCAC
AGAGGTGTTATCGCTGACAACCAAGCAAAATGGGCTGTCCCGACAACACGAACAGATGACAAG
TTGCGAATGGAGACATGCTTCCAACAGGCGTGTAAGGGTAAAATCCAAGCACTCTGCGAGAAT
CCCGAGTGGGCACCATTGAAGGATAACAGGATTCCTTCATACGGGGTCTTGTCTGTTGATCTG
AGTCTGACAGTTGAGCTTAAAATCAAAATTGCTTCGGGATTCGGGCCATTGATCACACACGGT
TCAGGGATGGACCTATACAAATCCAACCACAACAATGTGTATTGGCTGACTATCCCGCCAATG
AAGAACCTAGCCTTAGGTGTAATCAACACATTGGAGTGGATACCGAGATTCAAGGTTAGTCCC
GCGCTCTTCAATGTCCCAATTAAGGAAGCAGGCGGAGACTGCCATGCCCCAACATACCTACCT
GCGGAGGTGGATGGTGATGTCAAACTCAGTTCCAATCTGGTGATTCTACCTGGTCAAGATCTC
CAATATGTTTTGGCAACCTACGATACTTCCGCGGTTGAACATGCTGTGGTTTATTACGTTTAC
AGCCCAAGCCGCTCATTTTCTTACTTTTATCCTTTTAGGTTGCCTATAAAGGGGGTCCCCATC
GAATTACAAGTGGAATGCTTCACATGGGACCAAAAACTCTGGTGCCGTCACTTCTGTGTGCTT
GCGGACTCAGAATCTGGTGGACATATCACTCACTCTGGGATGGTGGGCATGGGAGTCAGCTGC
ACAGTCACCCGGGAAGATGGAACCAATGACTACAAAGACGATGACGACAAGTGA
>Exemplary mutant measles envelope protein (with leader
sequence), amino acid sequence (SEQ ID NO: 21):
MGSRIVINREHLMIDRPYVLLAVLFVMFLSLIGLLAIAGIRLHRAAIYTAEIHKSLSTNLDVT
NSIEHQVKDVLTPLFKIIGDEVGLRTPQRFTDLVKFISDKIKFLNPDREYDFRDLTWCINPPE
RIKLDYDQYCADVAAEELMNALVNSTLLETRTTNQFLAVSKGNCSGPTTIRGQFSNMSLSLLD
LYLGRGYNVSSIVTMTSQGMYGGTYLVEKPNLSSKRSELSQLSMYRVFEVGVIRNPGLGAPVF
HMTNYLEQPVSNDLSNCMVALGELKLAALCHGEDSITIPYQGSGKGVSFQLVKLGVWKSPTDM
QSWVPLSTDDPVIDRLYLSSHRGVIADNQAKWAVPTTRTDDKLRMETCFQQACKGKIQALCEN
PEWAPLKDNRIPSYGVLSVDLSLTVELKIKIASGFGPLITHGSGMDLYKSNHNNVYWLTIPPM
KNLALGVINTLEWIPRFKVSPALFNVPIKEAGGDCHAPTYLPAEVDGDVKLSSNLVILPGQDL
QYVLATYDTSAVEHAVVYYVYSPSRSFSYFYPFRLPIKGVPIELQVECFTWDQKLWCRHFCVL
ADSESGGHITHSGMVGMGVSCTVTREDGTNDYKDDDDK
>Exemplary mutant Nipah envelope protein, DNA
sequence (SEQ ID NO: 22):
ATGAAGAAGATCAACGAGGGCCTGCTGGACAGCAAGATCCTGAGCGCCTTCAACACCGTGATT
GCCCTGCTGGGCTCTATCGTGATCATCGTGATGAACATCATGATCATCCAGAACTACACCCGG
TCCACCGACAACCAGGCCGTGATTAAGGATGCTCTGCAGGGAATCCAGCAGCAGATCAAAGGC
CTGGCCGACAAGATCGGCACAGAGATCGGCCCTAAGGTGTCCCTGATCGACACCAGCAGCACC
ATCACAATCCCCGCCAATATCGGACTGCTGGGAAGCAAGATCAGCCAGAGCACCGCCAGCATC
AACGAGAACGTGAACGAGAAGTGCAAGTTCACCCTGCCTCCACTGAAGATCCACGAGTGCAAC
ATCAGCTGCCCCAATCCTCTGCCATTCAGAGAGTACAGACCCCAGACAGAGGGCGTGTCCAAT
CTCGTGGGCCTGCCTAACAACATCTGCCTGCAGAAAACCAGCAACCAGATCCTGAAGCCTAAG
CTGATCTCCTACACACTGCCCGTCGTGGGCCAGAGCGGCACCTGTATTACAGATCCTCTGCTG
GCCATGGACGAGGGCTACTTTGCCTACAGCCACCTGGAAAGAATCGGCAGCTGTAGCCGGGGA
GTGTCCAAGCAGAGAATCATCGGCGTGGGCGAAGTGCTGGATAGAGGCGACGAAGTGCCCAGC
CTGTTCATGACCAATGTGTGGACCCCTCCTAATCCTAACACCGTGTACCACTGCAGCGCCGTG
TACAACAACGAGTTCTACTACGTGCTGTGCGCCGTGTCCACAGTGGGCGACCCTATCCTGAAC
AGCACCTATTGGAGCGGCAGCCTGATGATGACCAGACTGGCCGTGAAGCCCAAGAGCAATGGC
GGCGGATACAACCAGCATCAGCTGGCCCTGCGGTCCATCGAGAAGGGCAGATACGACAAAGTG
ATGCCTTACGGCCCCAGCGGCATCAAGCAAGGCGATACCCTGTACTTTCCCGCCGTGGGATTT
CTCGTGCGGACCGAGTTCAAGTACAACGACAGCAACTGCCCCATCACCAAGTGCCAGTACAGC
AAGCCCGAGAACTGCAGACTGAGCATGGGCATCAGACCCAACAGCCACTACATCCTGAGAAGC
GGCCTGCTGAAGTACAACCTGAGCGACGGCGAGAACCCCAAGGTGGTGTTCATCGAGATCAGC
GACCAGCGGCTGTCTATCGGCAGCCCCTCCAAGATCTACGACTCTCTGGGCCAGCCAGTGTTC
TACCAGGCCAGCTTTAGCTGGGACACCATGATCAAGTTCGGCGACGTGCTGACCGTGAATCCC
CTGGTGGTCAACTGGCGGAACAATACCGTGATCAGCCGGCCTGGCCAGTCTCAGTGCCCCAGA
TTCAATACCTGTCCTGCCATTTGCGCCGAAGGCGTGTACAATGACGCCTTCCTGATCGATCGG
ATCAACTGGATCTCTGCCGGCGTGTTCCTGGACTCTAATGCCACAGCCGCCAATCCTGTGTTC
ACCGTGTTCAAGGACAATGAGATCCTGTATCGGGCCCAGCTGGCCTCCGAGGACACAAATGCC
CAGAAAACAATCACCAACTGCTTTCTGCTCAAGAACAAGATCTGGTGCATCAGCCTGGTGGAA
ATCTACGACACCGGCGACAACGTGATCAGGCCCAAGCTGTTCGCCGTGAAGATCCCTGAGCAG
TGTACAGGCGGCGGAGGATCTGGCGGAGGTGGAAGCGGAGGCGGTGGATCTGCTAGCGATTAC
AAGGATGACGACGATAAGTGA
>Exemplary mutant Nipah envelope protein, amino acid
sequence (SEQ ID NO: 23):
MKKINEGLLDSKILSAFNTVIALLGSIVIIVMNIMIIQNYTRSTDNQAVIKDALQGIQQQIKG
LADKIGTEIGPKVSLIDTSSTITIPANIGLLGSKISQSTASINENVNEKCKFTLPPLKIHECN
ISCPNPLPFREYRPQTEGVSNLVGLPNNICLQKTSNQILKPKLISYTLPVVGQSGTCITDPLL
AMDEGYFAYSHLERIGSCSRGVSKQRIIGVGEVLDRGDEVPSLFMTNVWTPPNPNTVYHCSAV
YNNEFYYVLCAVSTVGDPILNSTYWSGSLMMTRLAVKPKSNGGGYNQHQLALRSIEKGRYDKV
MPYGPSGIKQGDTLYFPAVGFLVRTEFKYNDSNCPITKCQYSKPENCRLSMGIRPNSHYILRS
GLLKYNLSDGENPKVVFIEISDQRLSIGSPSKIYDSLGQPVFYQASFSWDTMIKFGDVLTVNP
LVVNWRNNTVISRPGQSQCPRFNTCPAICAEGVYNDAFLIDRINWISAGVFLDSNATAANPVF
TVFKDNEILYRAQLASEDTNAQKTITNCFLLKNKIWCISLVEIYDTGDNVIRPKLFAVKIPEQ
CTGGGGSGGGGSGGGGSASDYKDDDDK
>Cocal Virus Glycoprotein, amino acid sequence:
(SEQ ID NO: 24):
MNFLLLTFIVLPLCSHAKFSIVFPQSQKGNWKNVPSSYHYCPSSSDQNWHNDLLGITMKVKMP
KTHKAIQADGWMCHAAKWITTCDFRWYGPKYITHSIHSIQPTSEQCKESIKQTKQGTWMSPGF
PPQNCGYATVTDSVAVVVQATPHHVLVDEYTGEWIDSQFPNGKCETEECETVHNSTVWYSDYK
VTGLCDATLVDTEITFFSEDGKKESIGKPNTGYRSNYFAYEKGDKVCKMNYCKHAGVRLPSGV
WFEFVDQDVYAAAKLPECPVGATISAPTQTSVDVSLILDVERILDYSLCQETWSKIRSKQPVS
PVDLSYLAPKNPGTGPAFTIINGTLKYFETRYIRIDIDNPIISKMVGKISGSQTERELWTEWF
PYEGVEIGPNGILKTPTGYKFPLFMIGHGMLDSDLHKTSQAEVFEHPHLAEAPKQLPEEETLF
FGDTGISKNPVELIEGWFSSWKSTVVTFFFAIGVFILLYVVARIVIAVRYRYQGSNNKRIYND
IEMSRFRK*
>Cocal Virus Glycoprotein, DNA sequence: (SEQ ID
NO: 25):
ATGAACTTTCTGCTGCTCACGTTTATCGTACTCCCGTTGTGCTCTCATGCGAAATTTTCAATA
GTCTTTCCTCAGTCCCAGAAAGGGAATTGGAAAAATGTTCCCTCCAGTTACCACTATTGTCCC
TCCTCCTCTGACCAAAACTGGCACAATGACTTGCTCGGGATTACAATGAAAGTAAAGATGCCG
AAAACCCATAAAGCCATACAGGCGGATGGGTGGATGTGTCACGCTGCGAAGTGGATCACTACA
TGCGATTTCCGGTGGTATGGCCCTAAGTACATTACACACTCTATCCATAGCATACAGCCGACA
TCAGAGCAATGCAAAGAGAGTATTAAACAGACCAAACAAGGGACATGGATGAGCCCTGGCTTT
CCACCTCAGAATTGTGGGTACGCGACCGTCACGGATAGTGTCGCTGTTGTGGTGCAGGCCACG
CCACATCACGTACTCGTAGATGAATATACTGGTGAATGGATCGACTCCCAATTCCCGAATGGG
AAATGTGAGACGGAAGAGTGCGAAACAGTGCATAACTCAACCGTTTGGTATTCCGATTACAAG
GTTACTGGTCTTTGCGACGCCACCCTCGTGGATACCGAGATCACGTTTTTTAGTGAGGATGGC
AAGAAAGAGTCAATAGGCAAACCTAATACTGGCTACCGGAGTAACTATTTCGCTTACGAGAAG
GGTGACAAGGTATGTAAAATGAACTATTGCAAGCATGCGGGAGTGCGACTCCCCAGTGGGGTA
TGGTTCGAATTTGTTGACCAAGACGTATACGCCGCTGCGAAGTTGCCAGAATGCCCCGTAGGC
GCGACCATTTCAGCACCTACCCAAACGTCCGTTGACGTCTCCTTGATACTGGATGTAGAGCGA
ATCCTGGACTACAGTCTCTGCCAGGAAACGTGGTCAAAAATAAGAAGTAAGCAGCCAGTTTCA
CCCGTGGATCTGTCTTATCTGGCGCCAAAAAACCCGGGCACGGGCCCTGCTTTTACCATAATT
AACGGAACGCTTAAATACTTCGAAACCCGCTACATTAGAATCGATATAGACAATCCTATTATC
AGCAAGATGGTAGGGAAGATATCTGGGTCTCAAACGGAGCGAGAATTGTGGACGGAGTGGTTC
CCTTATGAGGGAGTGGAAATTGGGCCCAACGGGATCCTCAAGACCCCAACGGGTTACAAGTTC
CCTCTGTTTATGATCGGCCATGGCATGTTGGACAGTGACTTGCACAAAACATCTCAGGCAGAG
GTTTTCGAACATCCACATTTGGCGGAGGCGCCCAAGCAACTTCCAGAAGAAGAAACTCTCTTC
TTTGGAGATACAGGCATTTCAAAAAATCCTGTAGAACTGATAGAAGGGTGGTTCTCTTCCTGG
AAATCAACGGTTGTCACGTTTTTCTTTGCAATAGGCGTATTTATACTCCTGTACGTCGTAGCC
CGCATTGTGATCGCAGTACGATACAGATACCAGGGCAGTAACAATAAACGCATATATAATGAC
ATCGAAATGTCAAGGTTCCGAAAGtga
>Cocal-dead (mutations to ablate native tropism
bolded in protein sequence; these are K64Q and R371A,
counting from the start codon), amino acid sequence:
(SEQ ID NO: 26):
MNFLLLTFIVLPLCSHAKFSIVFPQSQKGNWKNVPSSYHYCPSSSDQNWHNDLLGITMKVKMP
QTHKAIQADGWMCHAAKWITTCDFRWYGPKYITHSIHSIQPTSEQCKESIKQTKQGTWMSPGF
PPQNCGYATVTDSVAVVVQATPHHVLVDEYTGEWIDSQFPNGKCETEECETVHNSTVWYSDYK
VTGLCDATLVDTEITFFSEDGKKESIGKPNTGYRSNYFAYEKGDKVCKMNYCKHAGVRLPSGV
WFEFVDQDVYAAAKLPECPVGATISAPTQTSVDVSLILDVERILDYSLCQETWSKIRSKQPVS
PVDLSYLAPKNPGTGPAFTIINGTLKYFETRYIRIDIDNPIISKMVGKISGSQTEAELWTEWF
PYEGVEIGPNGILKTPTGYKFPLFMIGHGMLDSDLHKTSQAEVFEHPHLAEAPKQLPEEETLF
FGDTGISKNPVELIEGWFSSWKSTVVTFFFAIGVFILLYVVARIVIAVRYRYQGSNNKRIYND
IEMSRFRK
>Cocal-dead (mutations to ablate native tropism),
DNA sequence: (SEQ ID NO: 27):
ATGAACTTTCTGCTGCTCACGTTTATCGTACTCCCGTTGTGCTCTCATGCGAAATTTTCAATA
GTCTTTCCTCAGTCCCAGAAAGGGAATTGGAAAAATGTTCCCTCCAGTTACCACTATTGTCCC
TCCTCCTCTGACCAAAACTGGCACAATGACTTGCTCGGGATTACAATGAAAGTAAAGATGCCG
cagACCCATAAAGCCATACAGGCGGATGGGTGGATGTGTCACGCTGCGAAGTGGATCACTACA
TGCGATTTCCGGTGGTATGGCCCTAAGTACATTACACACTCTATCCATAGCATACAGCCGACA
TCAGAGCAATGCAAAGAGAGTATTAAACAGACCAAACAAGGGACATGGATGAGCCCTGGCTTT
CCACCTCAGAATTGTGGGTACGCGACCGTCACGGATAGTGTCGCTGTTGTGGTGCAGGCCACG
CCACATCACGTACTCGTAGATGAATATACTGGTGAATGGATCGACTCCCAATTCCCGAATGGG
AAATGTGAGACGGAAGAGTGCGAAACAGTGCATAACTCAACCGTTTGGTATTCCGATTACAAG
GTTACTGGTCTTTGCGACGCCACCCTCGTGGATACCGAGATCACGTTTTTTAGTGAGGATGGC
AAGAAAGAGTCAATAGGCAAACCTAATACTGGCTACCGGAGTAACTATTTCGCTTACGAGAAG
GGTGACAAGGTATGTAAAATGAACTATTGCAAGCATGCGGGAGTGCGACTCCCCAGTGGGGTA
TGGTTCGAATTTGTTGACCAAGACGTATACGCCGCTGCGAAGTTGCCAGAATGCCCCGTAGGC
GCGACCATTTCAGCACCTACCCAAACGTCCGTTGACGTCTCCTTGATACTGGATGTAGAGCGA
ATCCTGGACTACAGTCTCTGCCAGGAAACGTGGTCAAAAATAAGAAGTAAGCAGCCAGTTTCA
CCCGTGGATCTGTCTTATCTGGCGCCAAAAAACCCGGGCACGGGCCCTGCTTTTACCATAATT
AACGGAACGCTTAAATACTTCGAAACCCGCTACATTAGAATCGATATAGACAATCCTATTATC
AGCAAGATGGTAGGGAAGATATCTGGGTCTCAAACGGAGgccGAATTGTGGACGGAGTGGTTC
CCTTATGAGGGAGTGGAAATTGGGCCCAACGGGATCCTCAAGACCCCAACGGGTTACAAGTTC
CCTCTGTTTATGATCGGCCATGGCATGTTGGACAGTGACTTGCACAAAACATCTCAGGCAGAG
GTTTTCGAACATCCACATTTGGCGGAGGCGCCCAAGCAACTTCCAGAAGAAGAAACTCTCTTC
TTTGGAGATACAGGCATTTCAAAAAATCCTGTAGAACTGATAGAAGGGTGGTTCTCTTCCTGG
AAATCAACGGTTGTCACGTTTTTCTTTGCAATAGGCGTATTTATACTCCTGTACGTCGTAGCC
CGCATTGTGATCGCAGTACGATACAGATACCAGGGCAGTAACAATAAACGCATATATAATGAC
ATCGAAATGTCAAGGTTCCGAAAGtga
>mS4-3a-trunc-pStalk-PDGFR, amino acid sequence:
(SEQ ID NO: 28):
MKKTQTWIITCIYLQLLLFNPLVKTKEICGDPVTDNVKDITKLVANLPNDYMITLNYVAGMDV
LPSHCWLRDMVIQLSLSLTTLLDKFSNISEGLSNYSIIHKLGIIVDDLFFCMEENAPKNIKEF
PKRPETRSFTPEEFFSIFNRSIDAFKDFMVASDTSDCVLSYPYDVPDYAASAVGQDTQEVIVV
PHSLPFKVVVISAILALVVLTIISLIILIMLWQKKPR
>m54-3a-trunc-pStalk-PDGFR, DNA sequence: (SEQ
ID NO: 29):
tgtgtgctggcccatcactttggcaaagcacgtgagatctgaattctgacactATGAAAAAAA
CACAAACTTGGATCATTACTTGCATATACCTGCAACTTCTCCTTTTCAACCCACTCGTCAAGA
CCAAAGAAATATGCGGCGACCCCGTCACTGATAACGTGAAGGATATCACCAAACTCGTTGCTA
ACCTTCCAAATGACTACATGATTACATTGAACTATGTAGCAGGAATGGACGTTCTTCCATCAC
ATTGCTGGCTCCGGGACATGGTAATCCAGCTTAGCCTCAGCCTTACTACCTTGCTGGACAAGT
TTAGCAACATTTCCGAAGGGTTGAGTAACTATAGTATTATTCACAAGCTCGGTATCATAGTTG
ACGACTTGTTCTTCTGTATGGAAGAGAATGCACCCAAAAATATCAAAGAATTCCCCAAAAGGC
CCGAAACCAGGTCATTTACCCCAGAAGAATTTTTCAGTATTTTTAATCGCTCAATAGACGCAT
TCAAGGATTTCATGGTTGCTTCTGACACATCTGACTGCGTATTGTCCTATCCTTACGATGTCC
CGGACTATGCTGCTAGCGCTGTGGGCCAGGACACGCAGGAGGTCATCGTGGTGCCACACTCCT
TGCCCTTTAAGGTGGTGGTGATCTCAGCCATCCTGGCCCTGGTGGTGCTCACCATCATCTCCC
TTATCATCCTCATCATGCTTTGGCAGAAGAAGCCACGTtga
>mFLT3LG-pStalk-PDGFR, amino acid sequence: (SEQ
ID NO: 30):
MTVLAPAWSPNSSLLLLLLLLSPCLRGTPDCYFSHSPISSNFKVKFRELTDHLLKDYPVTVAV
NLQDEKHCKALWSLFLAQRWIEQLKTVAGSKMQTLLEDVNTEIHFVTSCTFQPLPECLRFVQT
NISHLLKDTCTQLLALKPCIGKACQNFSRCLEVQCQPDSSTLLPPRSPIALEATELPEPRPRQ
YPYDVPDYAASAVGQDTQEVIVVPHSLPFKVVVISAILALVVLTIISLIILIMLWQKKPR
>mFLT3LG-pStalk-PDGFR, DNA sequence: (SEQ ID NO: 31):
ATGACCGTACTTGCTCCAGCTTGGAGCCCTAACTCCTCTCTCCTTCTGCTGTTGCTGCTTCTG
TCCCCATGTCTGCGGGGTACCCCCGACTGTTATTTTTCTCATAGCCCAATATCTAGCAATTTC
AAAGTTAAGTTTCGGGAGCTTACCGATCATTTGCTTAAGGATTATCCAGTAACAGTAGCAGTT
AATCTCCAAGACGAGAAACACTGTAAGGCCTTGTGGTCCCTCTTTCTTGCCCAACGCTGGATT
GAGCAGCTTAAGACCGTAGCTGGCTCAAAAATGCAAACTCTCCTGGAGGATGTCAACACAGAG
ATTCATTTTGTCACCTCCTGCACCTTTCAACCTCTCCCTGAGTGCCTTAGATTCGTTCAGACT
AACATTTCTCACCTCCTGAAGGACACCTGCACCCAGCTGCTTGCTCTGAAACCTTGCATCGGC
AAGGCATGTCAAAATTTCTCACGGTGTCTCGAAGTCCAGTGCCAGCCTGATAGTTCCACATTG
CTCCCCCCAAGGTCACCCATAGCACTGGAAGCCACTGAACTTCCCGAACCACGCCCTCGGCAG
TATCCTTACGATGTCCCGGACTATGCTGCTAGCGCTGTGGGCCAGGACACGCAGGAGGTCATC
GTGGTGCCACACTCCTTGCCCTTTAAGGTGGTGGTGATCTCAGCCATCCTGGCCCTGGTGGTG
CTCACCATCATCTCCCTTATCATCCTCATCATGCTTTGGCAGAAGAAGCCACGTtga
>mSCFa-trunc-pStalk-PDGFR, amino acid sequence (SEQ ID
NO: 32):
MKKTQTWIITCIYLQLLLFNPLVKTKEICGNPVTDNVKDITKLVANLPNDYMITLNYVAGMDV
LPSHCWLRDMVIQLSLSLTTLLDKFSNISEGLSNYSIIDKLGKIVDDLVLCMEENAPKNIKES
PKRPETRSFTPEEFFSIFNRSIDAFKDFMVASDTSDCVLSYPYDVPDYAASAVGQDTQEVIVV
PHSLPFKVVVISAILALVVLTIISLIILIMLWQKKPR
>mSCFa-trunc-pStalk-PDGFR, DNA sequence (SEQ ID NO: 33):
atgaaaaaaacccagacctggattattacctgcatttatctgcagctgctgctgtttaacccg
ctggtgaaaaccaaagaaatttgcggcaacccggtgaccgataacgtgaaagatattaccaaa
ctggtggcgaacctgccgaacgattatatgattaccctgaactatgtggcgggcatggatgtg
ctgccgagccattgctggctgcgcgatatggtgattcagctgagcctgagcctgaccaccctg
ctggataaatttagcaacattagcgaaggcctgagcaactatagcattattgataaactgggc
aaaattgtggatgatctggtgctgtgcatggaagaaaacgcgccgaaaaacattaaagaaagc
ccgaaacgcccggaaacccgcagctttaccccggaagaattttttagcatttttaaccgcagc
attgatgcgtttaaagattttatggtggcgagcgataccagcgattgcgtgctgagctatccg
tatgatgtgccggattatgcggcgagcgcggtgggccaggatacccaggaagtgattgtggtg
ccgcatagcctgccgtttaaagtggtggtgattagcgcgattctggcgctggtggtgctgacc
attattagcctgattattctgattatgctgtggcagaaaaaaccgcgc
>mTPO-trunc-pStalk-PDGFR, amino acid sequence (SEQ ID
NO: 34):
MELTDLLLAAMLLAVARLTLSSPVAPACDPRLLNKLLRDSHLLHSRLSQCPDVDPLSIPVLLP
AVDFSLGEWKTQTEQSKAQDILGAVSLLLEGVMAARGQLEPSCLSSLLGQLSGQVRLLLGALQ
GLLGTQLPLQGRTTAHKDPNALFLSLQQLLRGKVRFLLLVEGPTLCVRYPYDVPDYAASAVGQ
DTQEVIVVPHSLPFKVVVISAILALVVLTIISLIILIMLWQKKPR
>mTPO-trunc-pStalk-PDGFR, DNA sequence (SEQ ID NO: 35):
ATGGAATTGACTGACCTGCTGTTGGCTGCCATGCTTCTTGCCGTCGCCCGCTTGACACTCAGC
TCTCCAGTTGCTCCCGCCTGCGATCCCAGGTTGCTTAACAAACTGCTTCGAGACTCTCATCTG
CTTCACAGCAGGTTGTCTCAATGTCCAGACGTGGATCCACTTTCTATTCCTGTCCTGCTGCCC
GCAGTTGACTTCTCATTGGGAGAGTGGAAAACTCAGACCGAACAATCTAAGGCACAAGACATA
TTGGGCGCTGTGTCTCTGTTGCTCGAAGGCGTCATGGCTGCCCGGGGGCAGCTTGAACCCTCA
TGTCTCTCCTCCTTGCTGGGTCAGCTTTCTGGACAAGTTAGATTGCTGCTGGGAGCTTTGCAA
GGGTTGTTGGGTACACAACTCCCACTTCAGGGTCGCACTACCGCTCACAAAGATCCAAATGCC
CTTTTTCTTAGTCTTCAACAATTGCTGCGGGGAAAAGTGAGATTTTTGTTGCTGGTTGAAGGA
CCAACATTGTGCGTTCGATATCCTTACGATGTCCCGGACTATGCTGCTAGCGCTGTGGGCCAG
GACACGCAGGAGGTCATCGTGGTGCCACACTCCTTGCCCTTTAAGGTGGTGGTGATCTCAGCC
ATCCTGGCCCTGGTGGTGCTCACCATCATCTCCCTTATCATCCTCATCATGCTTTGGCAGAAG
AAGCCACGTtga
>mS4-3a-IgG4hinge-PDGFR, amino acid (SEQ ID NO: 36)
MKKTQTWIITCIYLQLLLFNPLVKTKEICGDPVTDNVKDITKLVANLPNDYMITLNYVAGMDV
LPSHCWLRDMVIQLSLSLTTLLDKFSNISEGLSNYSIIHKLGIIVDDLFFCMEENAPKNIKEF
PKRPETRSFTPEEFFSIFNRSIDAFKDFMVASDTSDCVLSYPYDVPDYAASESKYGPPCPPCP
AVGQDTQEVIVVPHSLPFKVVVISAILALVVLTIISLIILIMLWQKKPR*
>mS4-3a-IgG4hinge-PDGFR, DNA (SEQ ID NO: 37)
ATGAAAAAAACACAAACTTGGATCATTACTTGCATATACCTGCAACTTCTCCTTTTCAACCCA
CTCGTCAAGACCAAAGAAATATGCGGCGACCCCGTCACTGATAACGTGAAGGATATCACCAAA
CTCGTTGCTAACCTTCCAAATGACTACATGATTACATTGAACTATGTAGCAGGAATGGACGTT
CTTCCATCACATTGCTGGCTCCGGGACATGGTAATCCAGCTTAGCCTCAGCCTTACTACCTTG
CTGGACAAGTTTAGCAACATTTCCGAAGGGTTGAGTAACTATAGTATTATTCACAAGCTCGGT
ATCATAGTTGACGACTTGTTCTTCTGTATGGAAGAGAATGCACCCAAAAATATCAAAGAATTC
CCCAAAAGGCCCGAAACCAGGTCATTTACCCCAGAAGAATTTTTCAGTATTTTTAATCGCTCA
ATAGACGCATTCAAGGATTTCATGGTTGCTTCTGACACATCTGACTGCGTATTGTCCTATCCT
TACGATGTCCCGGACTATGCTGCTAGCGAAAGCAAGTATGGTCCTCCCTGCCCCCCGTGCCCA
GCTGTGGGCCAGGACACGCAGGAGGTCATCGTGGTGCCACACTCCTTGCCCTTTAAGGTGGTG
GTGATCTCAGCCATCCTGGCCCTGGTGGTGCTCACCATCATCTCCCTTATCATCCTCATCATG
CTTTGGCAGAAGAAGCCACGTtga
>mFLT3LG-IgG4hinge-PDGFR, amino acid (SEQ ID NO: 38)
MTVLAPAWSPNSSLLLLLLLLSPCLRGTPDCYFSHSPISSNFKVKFRELTDHLLKDYPVTVAV
NLQDEKHCKALWSLFLAQRWIEQLKTVAGSKMQTLLEDVNTEIHFVTSCTFQPLPECLRFVQT
NISHLLKDTCTQLLALKPCIGKACQNFSRCLEVQCQPDSSTLLPPRSPIALEATELPEPRPRQ
YPYDVPDYAASESKYGPPCPPCPAVGQDTQEVIVVPHSLPFKVVVISAILALVVLTIISLIIL
IMLWQKKPR*
>mFLT3LG-IgG4hinge-PDGFR, DNA (SEQ ID NO: 39)
ATGACCGTACTTGCTCCAGCTTGGAGCCCTAACTCCTCTCTCCTTCTGCTGTTGCTGCTTCTG
TCCCCATGTCTGCGGGGTACCCCCGACTGTTATTTTTCTCATAGCCCAATATCTAGCAATTTC
AAAGTTAAGTTTCGGGAGCTTACCGATCATTTGCTTAAGGATTATCCAGTAACAGTAGCAGTT
AATCTCCAAGACGAGAAACACTGTAAGGCCTTGTGGTCCCTCTTTCTTGCCCAACGCTGGATT
GAGCAGCTTAAGACCGTAGCTGGCTCAAAAATGCAAACTCTCCTGGAGGATGTCAACACAGAG
ATTCATTTTGTCACCTCCTGCACCTTTCAACCTCTCCCTGAGTGCCTTAGATTCGTTCAGACT
AACATTTCTCACCTCCTGAAGGACACCTGCACCCAGCTGCTTGCTCTGAAACCTTGCATCGGC
AAGGCATGTCAAAATTTCTCACGGTGTCTCGAAGTCCAGTGCCAGCCTGATAGTTCCACATTG
CTCCCCCCAAGGTCACCCATAGCACTGGAAGCCACTGAACTTCCCGAACCACGCCCTCGGCAG
TATCCTTACGATGTCCCGGACTATGCTGCTAGCGAAAGCAAGTATGGTCCTCCCTGCCCCCCG
TGCCCAGCTGTGGGCCAGGACACGCAGGAGGTCATCGTGGTGCCACACTCCTTGCCCTTTAAG
GTGGTGGTGATCTCAGCCATCCTGGCCCTGGTGGTGCTCACCATCATCTCCCTTATCATCCTC
ATCATGCTTTGGCAGAAGAAGCCACGTtga
>mTPO-trunc-IgG4hinge-PDGFR, amino acid (SEQ ID
NO: 40)
MELTDLLLAAMLLAVARLTLSSPVAPACDPRLLNKLLRDSHLLHSRLSQCPDVDPLSIPVLLP
AVDFSLGEWKTQTEQSKAQDILGAVSLLLEGVMAARGQLEPSCLSSLLGQLSGQVRLLLGALQ
GLLGTQLPLQGRTTAHKDPNALFLSLQQLLRGKVRFLLLVEGPTLCVRYPYDVPDYAASESKY
GPPCPPCPAVGQDTQEVIVVPHSLPFKVVVISAILALVVLTIISLIILIMLWQKKPR*
>mTPO-trunc-IgG4hinge-PDGFR, DNA (SEQ ID NO: 41)
ATGGAATTGACTGACCTGCTGTTGGCTGCCATGCTTCTTGCCGTCGCCCGCTTGACACTCAGC
TCTCCAGTTGCTCCCGCCTGCGATCCCAGGTTGCTTAACAAACTGCTTCGAGACTCTCATCTG
CTTCACAGCAGGTTGTCTCAATGTCCAGACGTGGATCCACTTTCTATTCCTGTCCTGCTGCCC
GCAGTTGACTTCTCATTGGGAGAGTGGAAAACTCAGACCGAACAATCTAAGGCACAAGACATA
TTGGGCGCTGTGTCTCTGTTGCTCGAAGGCGTCATGGCTGCCCGGGGGCAGCTTGAACCCTCA
TGTCTCTCCTCCTTGCTGGGTCAGCTTTCTGGACAAGTTAGATTGCTGCTGGGAGCTTTGCAA
GGGTTGTTGGGTACACAACTCCCACTTCAGGGTCGCACTACCGCTCACAAAGATCCAAATGCC
CTTTTTCTTAGTCTTCAACAATTGCTGCGGGGAAAAGTGAGATTTTTGTTGCTGGTTGAAGGA
CCAACATTGTGCGTTCGATATCCTTACGATGTCCCGGACTATGCTGCTAGCGAAAGCAAGTAT
GGTCCTCCCTGCCCCCCGTGCCCAGCTGTGGGCCAGGACACGCAGGAGGTCATCGTGGTGCCA
CACTCCTTGCCCTTTAAGGTGGTGGTGATCTCAGCCATCCTGGCCCTGGTGGTGCTCACCATC
ATCTCCCTTATCATCCTCATCATGCTTTGGCAGAAGAAGCCACGTtga
>mSCFa-IgG4hinge-PDGFR, amino acid (SEQ ID NO: 42)
MKKTQTWIITCIYLQLLLFNPLVKTKEICGNPVTDNVKDITKLVANLPNDYMITLNYVAGMDV
LPSHCWLRDMVIQLSLSLTTLLDKFSNISEGLSNYSIIDKLGKIVDDLVLCMEENAPKNIKES
PKRPETRSFTPEEFFSIFNRSIDAFKDFMVASDTSDCVLSYPYDVPDYAASESKYGPPCPPCP
AVGQDTQEVIVVPHSLPFKVVVISAILALVVLTIISLIILIMLWQKKPR*
>mSCFa-IgG4hinge-PDGFR, DNA (SEQ ID NO: 43)
ATGAAAAAAACACAAACTTGGATCATTACTTGCATATACCTGCAACTTCTCCTTTTCAACCCA
CTCGTCAAGACCAAAGAAATATGCGGCAACCCCGTCACTGATAACGTGAAGGATATCACCAAA
CTCGTTGCTAACCTTCCAAATGACTACATGATTACATTGAACTATGTAGCAGGAATGGACGTT
CTTCCATCACATTGCTGGCTCCGGGACATGGTAATCCAGCTTAGCCTCAGCCTTACTACCTTG
CTGGACAAGTTTAGCAACATTTCCGAAGGGTTGAGTAACTATAGTATTATTGATAAGCTCGGT
AAGATAGTTGACGACTTGGTTCTCTGTATGGAAGAGAATGCACCCAAAAATATCAAAGAATCC
CCCAAAAGGCCCGAAACCAGGTCATTTACCCCAGAAGAATTTTTCAGTATTTTTAATCGCTCA
ATAGACGCATTCAAGGATTTCATGGTTGCTTCTGACACATCTGACTGCGTATTGTCCTATCCT
TACGATGTCCCGGACTATGCTGCTAGCGAAAGCAAGTATGGTCCTCCCTGCCCCCCGTGCCCA
GCTGTGGGCCAGGACACGCAGGAGGTCATCGTGGTGCCACACTCCTTGCCCTTTAAGGTGGTG
GTGATCTCAGCCATCCTGGCCCTGGTGGTGCTCACCATCATCTCCCTTATCATCCTCATCATG
CTTTGGCAGAAGAAGCCACGTtga
>hSCFa-trunc-pStalk-PDGFR, amino acid (SEQ ID NO: 44)
MKKTQTWILTCIYLQLLLFNPLVKTEGICRNRVTNNVKDVTKLVANLPKDYMITLKYVPGMDV
LPSHCWISEMVVQLSDSLTDLLDKFSNISEGLSNYSIIDKLVNIVDDLVECVKENSSKDLKKS
FKSPEPRLFTPEEFFRIFNRSIDAFKDFVVASETSDCVVSYPYDVPDYAASAVGQDTQEVIVV
PHSLPFKVVVISAILALVVLTIISLIILIMLWQKKPR*
>hSCFa-trunc-pStalk-PDGFR, DNA (SEQ ID NO: 45)
TGAAGAAGACTCAGACCTGGATTCTGACGTGCATATATCTCCAACTCTTGCTTTTTAATCCCT
TGGTTAAGACCGAGGGGATTTGTCGGAACAGGGTGACTAACAACGTGAAAGATGTGACCAAAC
TGGTGGCAAACCTCCCGAAGGACTACATGATTACACTCAAATATGTGCCGGGCATGGATGTCT
TGCCAAGCCACTGTTGGATCTCCGAAATGGTTGTCCAGTTGTCCGACAGCCTTACGGATCTCC
TGGATAAATTTAGCAACATTAGCGAAGGTCTTTCTAATTATTCCATTATAGATAAACTCGTTA
ATATTGTAGATGACCTCGTCGAATGTGTGAAGGAAAATTCTAGCAAGGATTTGAAAAAATCCT
TTAAGTCACCGGAACCCCGACTTTTCACCCCCGAAGAATTTTTCCGAATATTCAACAGGAGCA
TAGATGCTTTCAAAGACTTCGTAGTGGCCAGCGAAACAAGTGACTGCGTGGTTTCCTATCCTT
ACGATGTCCCGGACTATGCTGCTAGCGCTGTGGGCCAGGACACGCAGGAGGTCATCGTGGTGC
CACACTCCTTGCCCTTTAAGGTGGTGGTGATCTCAGCCATCCTGGCCCTGGTGGTGCTCACCA
TCATCTCCCTTATCATCCTCATCATGCTTTGGCAGAAGAAGCCACGTtga
>hSCFa-trunc-IgG4hinge-PDGFR, amino acid (SEQ ID
NO: 46)
MKKTQTWILTCIYLQLLLFNPLVKTEGICRNRVTNNVKDVTKLVANLPKDYMITLKYVPGMDV
LPSHCWISEMVVQLSDSLTDLLDKFSNISEGLSNYSIIDKLVNIVDDLVECVKENSSKDLKKS
FKSPEPRLFTPEEFFRIFNRSIDAFKDFVVASETSDCVVSYPYDVPDYAASESKYGPPCPPCP
AVGQDTQEVIVVPHSLPFKVVVISAILALVVLTIISLIILIMLWQKKPR*
>hSCFa-trunc-IgG4hinge-PDGFR, DNA (SEQ ID NO: 47)
ATGAAGAAGACTCAGACCTGGATTCTGACGTGCATATATCTCCAACTCTTGCTTTTTAATCCC
TTGGTTAAGACCGAGGGGATTTGTCGGAACAGGGTGACTAACAACGTGAAAGATGTGACCAAA
CTGGTGGCAAACCTCCCGAAGGACTACATGATTACACTCAAATATGTGCCGGGCATGGATGTC
TTGCCAAGCCACTGTTGGATCTCCGAAATGGTTGTCCAGTTGTCCGACAGCCTTACGGATCTC
CTGGATAAATTTAGCAACATTAGCGAAGGTCTTTCTAATTATTCCATTATAGATAAACTCGTT
AATATTGTAGATGACCTCGTCGAATGTGTGAAGGAAAATTCTAGCAAGGATTTGAAAAAATCC
TTTAAGTCACCGGAACCCCGACTTTTCACCCCCGAAGAATTTTTCCGAATATTCAACAGGAGC
ATAGATGCTTTCAAAGACTTCGTAGTGGCCAGCGAAACAAGTGACTGCGTGGTTTCCTATCCT
TACGATGTCCCGGACTATGCTGCTAGCGAAAGCAAGTATGGTCCTCCCTGCCCCCCGTGCCCA
GCTGTGGGCCAGGACACGCAGGAGGTCATCGTGGTGCCACACTCCTTGCCCTTTAAGGTGGTG
GTGATCTCAGCCATCCTGGCCCTGGTGGTGCTCACCATCATCTCCCTTATCATCCTCATCATG
CTTTGGCAGAAGAAGCCACGTtga
>hFLT3LG-trunc-IgG4hinge-PDGFR, amino acid (SEQ
ID NO: 48)
MTVLAPAWSPTTYLLLLLLLSSGLSGTQDCSFQHSPISSDFAVKIRELSDYLLQDYPVTVASN
LQDEELCGGLWRLVLAQRWMERLKTVAGSKMQGLLERVNTEIHFVTKCAFQPPPSCLRFVQTN
ISRLLQETSEQLVALKPWITRQNFSRCLELQCQPDSSTLPPPWSPRPLEATAPTAPQPYPYDV
PDYAASESKYGPPCPPCPAVGQDTQEVIVVPHSLPFKVVVISAILALVVLTIISLIILIMLWQ
KKPR*
>hFLT3LG-trunc-IgG4hinge-PDGFR, DNA (SEQ ID NO: 49)
ATGACAGTGCTGGCCCCAGCCTGGAGTCCAACAACCTACCTTCTCTTGCTCTTGCTTCTTTCC
AGTGGCCTGTCAGGCACGCAAGATTGTTCATTTCAACATTCACCCATCAGTTCAGACTTTGCT
GTTAAAATTAGGGAGTTGAGCGATTACCTCCTGCAAGATTATCCTGTGACTGTTGCAAGCAAC
CTTCAGGATGAAGAGCTTTGCGGGGGGCTCTGGCGCCTCGTGTTGGCTCAGCGGTGGATGGAA
CGCCTCAAAACGGTGGCGGGTAGTAAGATGCAGGGTCTGTTGGAGAGAGTTAACACGGAGATC
CATTTCGTAACCAAGTGTGCATTTCAACCGCCACCCTCTTGCCTTAGATTTGTCCAAACCAAT
ATCAGCCGACTTCTCCAAGAGACATCTGAACAGCTTGTTGCCCTGAAACCGTGGATTACAAGG
CAAAACTTTTCACGCTGCTTGGAGCTTCAATGTCAACCTGACAGTAGTACCCTTCCGCCTCCT
TGGTCTCCTAGACCGCTTGAAGCTACGGCTCCTACGGCACCACAACCCTATCCTTACGATGTC
CCGGACTATGCTGCTAGCGAAAGCAAGTATGGTCCTCCCTGCCCCCCGTGCCCAGCTGTGGGC
CAGGACACGCAGGAGGTCATCGTGGTGCCACACTCCTTGCCCTTTAAGGTGGTGGTGATCTCA
GCCATCCTGGCCCTGGTGGTGCTCACCATCATCTCCCTTATCATCCTCATCATGCTTTGGCAG
AAGAAGCCACGTtga
>hFLT3LG-pStalk-PDGFR, amino acid (SEQ ID NO: 50)
MTVLAPAWSPTTYLLLLLLLSSGLSGTQDCSFQHSPISSDFAVKIRELSDYLLQDYPVTVASN
LQDEELCGGLWRLVLAQRWMERLKTVAGSKMQGLLERVNTEIHFVTKCAFQPPPSCLRFVQTN
ISRLLQETSEQLVALKPWITRQNFSRCLELQCQPDSSTLPPPWSPRPLEATAPTAPQPYPYDV
PDYAASAVGQDTQEVIVVPHSLPFKVVVISAILALVVLTIISLIILIMLWQKKPR*
>hFLT3LG-pStalk-PDGFR, DNA (SEQ ID NO: 51)
ATGACAGTGCTGGCCCCAGCCTGGAGTCCAACAACCTACCTTCTCTTGCTCTTGCTTCTTTCC
AGTGGCCTGTCAGGCACGCAAGATTGTTCATTTCAACATTCACCCATCAGTTCAGACTTTGCT
GTTAAAATTAGGGAGTTGAGCGATTACCTCCTGCAAGATTATCCTGTGACTGTTGCAAGCAAC
CTTCAGGATGAAGAGCTTTGCGGGGGGCTCTGGCGCCTCGTGTTGGCTCAGCGGTGGATGGAA
CGCCTCAAAACGGTGGCGGGTAGTAAGATGCAGGGTCTGTTGGAGAGAGTTAACACGGAGATC
CATTTCGTAACCAAGTGTGCATTTCAACCGCCACCCTCTTGCCTTAGATTTGTCCAAACCAAT
ATCAGCCGACTTCTCCAAGAGACATCTGAACAGCTTGTTGCCCTGAAACCGTGGATTACAAGG
CAAAACTTTTCACGCTGCTTGGAGCTTCAATGTCAACCTGACAGTAGTACCCTTCCGCCTCCT
TGGTCTCCTAGACCGCTTGAAGCTACGGCTCCTACGGCACCACAACCCTATCCTTACGATGTC
CCGGACTATGCTGCTAGCGCTGTGGGCCAGGACACGCAGGAGGTCATCGTGGTGCCACACTCC
TTGCCCTTTAAGGTGGTGGTGATCTCAGCCATCCTGGCCCTGGTGGTGCTCACCATCATCTCC
CTTATCATCCTCATCATGCTTTGGCAGAAGAAGCCACGTtga
>mouse m54-3a-truncated (SEQ ID NO: 54)
KEICGDPVTDNVKDITKLVANLPNDYMITLNYVAGMDVLPSHCWLRDMVIQLSLSLTTLLDKF
SNISEGLSNYSIIHKLGIIVDDLFFCMEENAPKNIKEFPKRPETRSFTPEEFFSIFNRSIDAF
KDFMVASDTSDCVLS
>mouse FLT3 ligand (SEQ ID NO: 55)
GTPDCYFSHSPISSNFKVKFRELTDHLLKDYPVTVAVNLQDEKHCKALWSLFLAQRWIEQLKT
VAGSKMQTLLEDVNTEIHFVTSCTFQPLPECLRFVQTNISHLLKDTCTQLLALKPCIGKACQN
FSRCLEVQCQPDSSTLLPPRSPIALEATELPEPRPRQ
>mouse SCFa-truncated (SEQ ID NO: 56)
KEICGNPVTDNVKDITKLVANLPNDYMITLNYVAGMDVLPSHCWLRDMVIQLSLSLTTLLDKF
SNISEGLSNYSIIDKLGKIVDDLVLCMEENAPKNIKESPKRPETRSFTPEEFFSIFNRSIDAF
KDFMVASDTSDCVLS
>Mouse TPO truncated (SEQ ID NO: 57)
SPVAPACDPRLLNKLLRDSHLLHSRLSQCPDVDPLSIPVLLPAVDFSLGEWKTQTEQSKAQDI
LGAVSLLLEGVMAARGQLEPSCLSSLLGQLSGQVRLLLGALQGLLGTQLPLQGRTTAHKDPNA
LFLSLQQLLRGKVRFLLLVEGPTLCVR
>human SCFa truncated (SEQ ID NO: 58)
EGICRNRVTNNVKDVTKLVANLPKDYMITLKYVPGMDVLPSHCWISEMVVQLSDSLTDLLDKF
SNISEGLSNYSIIDKLVNIVDDLVECVKENSSKDLKKSFKSPEPRLFTPEEFFRIFNRSIDAF
KDFVVASETSDCVVS
>Human FLT3 ligand (SEQ ID NO: 59)
QDCSFQHSPISSDFAVKIRELSDYLLQDYPVTVASNLQDEELCGGLWRLVLAQRWMERLKTVA
GSKMQGLLERVNTEIHFVTKCAFQPPPSCLRFVQTNISRLLQETSEQLVALKPWITRQNFSRC
LELQCQPDSSTLPPPWSPRPLEATAPTAPQP

1. A method of delivering one or more nucleic acids to a hematopoietic stem cell (HSC), the method comprising:
(i) providing a retrovirus comprising the one or more nucleic acids, a viral envelope protein comprising at least one mutation that diminishes its native function, and a non-viral membrane-bound protein comprising an extracellular targeting domain that binds to a protein on the surface of the HSC; and
(ii) contacting the retrovirus with the HSC,
thereby delivering the one or more nucleic acids to the HSC.

2. The method of claim 1, wherein the extracellular targeting domain is stem cell factor (SCF), FMS-like tyrosine kinase 3 ligand (FLT3L), or thrombopoietin (TPO).

3. The method of claim 1, wherein the protein on the surface of the HSC is CD34, CD90, CD133, CD49f, CD201, c-Kit, FMS-like tyrosine kinase 3 (FLT3), or thrombopoietin receptor.

4. The method of claim 1, wherein the extracellular targeting domain comprises an amino acid sequence set forth in any one of SEQ ID NOs. 54-59.

5. The method of claim 1, wherein at least one of the one or more nucleic acids encodes a gene of interest, optionally wherein the gene of interest encodes a protein of interest.

6. The method of claim 5, wherein the protein of interest is a gene editing protein.

7. The method of claim 6, wherein the gene editing protein is a Cas endonuclease, a zinc finger nuclease, a transcription activator-like effector nuclease (TALEN), or a meganuclease, optionally wherein the Cas endonuclease is a Cas9 endonuclease.

8. The method of claim 1, wherein at least one of the one or more nucleic acids is a guide RNA.

9. The method of claim 1, wherein the retrovirus enters or infects the cell during (ii).

10. The method of claim 1, wherein the retrovirus is a lentivirus.

11. The method of claim 1, wherein the viral envelope protein is a VSV-G envelope protein, a measles virus envelope protein, a nipah virus envelope protein, or a cocal virus G protein.

12. The method of claim 11, where the at least one mutation of a VSV-G envelope protein is a mutation selected from the group consisting of H8, 141, K47, Y209, and R354.

13. The method of claim 11, where the viral envelope protein comprises a VSV-G envelope protein comprising the amino acid sequence set forth in SEQ ID NO: 16 or SEQ ID NO: 17.

14. The method of claim 11, where the at least one mutation of the measles virus envelope protein is a mutation selected from the group consisting of Y481, R533, 5548, and F549.

15. The method of claim 11, where the viral envelope protein comprises a measles virus envelope protein comprising the amino acid sequence set forth in SEQ ID NO: 21.

16. The method of claim 11, where the at least one mutation of the nipah virus envelope protein is a mutation selected from the group consisting of E501, W504, Q530, and E533.

17. The method of claim 11, where the viral envelope protein comprises a nipah virus envelope protein comprising the amino acid sequence set forth in SEQ ID NO: 23.

18. The method of claim 11, where the at least one mutation of the cocal virus G protein is a mutation selected from the group consisting of K64 and R371.

19. The method of claim 11, where the viral envelope protein comprises a cocal virus G protein comprising the amino acid sequence set forth in SEQ ID NO: 26.

20. The method of claim 1, wherein a linker is positioned between the membrane-bound domain and the extracellular targeting domain.

21. The method of claim 20, wherein:
(i) the linker is a rigid linker, optionally comprising a PDGFR stalk or a CD8αstalk;
(ii) the linker is a flexible linker, optionally comprising an amino acid sequence comprising GAPGAS (SEQ ID NO: 5) or GGGGS (SEQ ID NO: 7); or
(iii) the linker is an oligomerized linker, optionally comprising an IgG4 hinge or an amino acid sequence that can form a tetrameric coiled coil.

22.-23. (canceled)

24. The method of claim 1, wherein the HSC is a murine HSC or a human HSC.

25. The method of claim 1, wherein the one or more nucleic acids encode a chimeric antigen receptor.

26. A method of gene editing in a hematopoietic stem cell (HSC), the method comprising:
(i) providing a retrovirus comprising one or more nucleic acids encoding a gene editing composition, a viral envelope protein comprising at least one mutation that diminishes its native function, and a non-viral membrane-bound protein comprising an extracellular targeting domain that binds to a protein on the surface of the HSC; and
(ii) contacting the retrovirus with the HSC such that the one or more nucleic acids encoding a gene editing composition are delivered to the HSC,
wherein the gene editing composition specifically targets a section of the chromosomal DNA of the HSC to cause a genetic modification.

27.-48. (canceled)

49. The method of claim 3, wherein the extracellular targeting domain is an antibody, or binding fragment thereof, that binds to CD34, CD90, CD133, CD49f, CD201, c-Kit, FMS-like tyrosine kinase 3 (FLT3), or thrombopoietin receptor.
